(19) United States
(12) Patent Application Publication
(10) Pub. No.: US 2025/0027937 A1
(43) Pub. Date: Jan. 23, 2025

(54) DNA mapping and sequencing on linearized DNA molecules

(71) Applicant: Drexel University, Philadelphia, PA (US)

(72) Inventors: Ming Xiao, Huntingdon Valley, PA (US); Dharma Teja Varapula, Philadelphia, PA (US); Eric Michael LaBouff, Philadelphia, PA (US); Moses Noh, Ambler, PA (US)

(21) Appl. No.: 18/644,807

(22) Filed: Apr. 24, 2024

Related U.S. Application Data
(63) Continuation of application No. 16/945,638, filed on Jul. 31, 2020, now abandoned.
(60) Provisional application No. 62/881,776, filed on Aug. 1, 2019.

(51) Int. Cl. G01N 33/552; B01J 19/00; C12N 9/22; C12N 15/10; C12N 15/113; C12Q 1/6874

(52) U.S. Cl. CPC G01N 33/552; B01J 19/0046; C12N 9/22; C12N 15/1065; C12N 15/113; C12Q 1/6874; B01J 2219/00277; B01L 2300/163; B01L 2400/0406; C12N 2310/20; C12N 2800/80; C12Q 2523/303

(57) ABSTRACT

The present invention provides novel methods for immobilizing and/or optically mapping oligonucleotides, the method comprising immobilizing the oligonucleotides on a micropatterned substrate. In another aspect, the invention provides a method for mapping a genome, wherein the method is capable of resolving a single nucleotide polymorphism (SNP), the method comprising introducing to the genome a CRISPR/Cas9 system comprising at least one single-guide RNA (sgRNA) specific for a target sequence or a plurality of target sequences across the genome and a Cas9 D10A, wherein the CRISPR/Cas9 system nick labels the target sequence, and the target sequence or genome is analyzed.

### CROSS-REFERENCE TO RELATED APPLICATIONS

The present application is a continuation of U.S. patent application Ser. No. 16/945,638, filed Jul. 31, 2020, which claims priority under 35 U.S.C. § 119(e) to U.S. Provisional Patent Application No. 62/881,776, filed Aug. 1, 2019. The contents of each of these applications are hereby incorporated by reference in their entireties herein.

### STATEMENT REGARDING FEDERALLY SPONSORED RESEARCH OR DEVELOPMENT

This invention was made with government support under Grant No. R01-HG005946 awarded by the National Institutes of Health (NIH). The government has certain rights in the invention.

### INCORPORATION-BY-REFERENCE OF SEQUENCE LISTING

The instant application contains a Sequence Listing which has been submitted in XML format via Patent Center and is hereby incorporated by reference in its entirety. Said XML copy, created on Apr. 24, 2024, is named 046528-7097US2 Sequence Listing.xml and is 602,112 bytes in size.

### BACKGROUND OF THE INVENTION

Single DNA molecules when stretched out can provide a wide window to genomic data. Although commercial devices to stretch single DNA molecules exist, the length of linearized DNA achieved is still short for efficient large-scale genome assembly via sequence mapping. There is a need in the art for new devices and methods that are useful for immobilizing and linearizing oligonucleotides and/or for the interrogation of immobilized oligonucleotides. This disclosure addresses that need.

Further, restriction mapping has been applied in human genomics for physical mapping of genome fragments based on restriction enzyme cutting and was used extensively during the Human Genome Project to guide genome assembly. However, traditional restriction mapping is highly labor-intensive and requires large amounts of sample. More importantly, a traditional restriction map provides a “fingerprint” of the genomic DNA, not an ordered sequence of restriction sites. Therefore, there is a need in the art for DNA mapping methodologies that overcome the drawbacks of the currently practiced mapping techniques, the present invention addresses this need.

### SUMMARY OF THE INVENTION

In one aspect, the invention provides a method of immobilizing and linearizing an oligonucleotide, wherein the method comprises providing a micropatterned substrate, wherein the micropatterned substrate comprises at least one binding region having a first width; and at least one non-binding region having a second width; contacting the micropatterned substrate with a solution comprising a at least one oligonucleotide molecule, wherein one end of at least one oligonucleotide molecule attaches to the binding region of the micropatterned substrate; and combing the at least one oligonucleotide molecule such that the at least one oligonucleotide molecule extends from the binding region into at least a portion of an adjacent non-binding region; thereby immobilizing and linearizing the at least one oligonucleotide molecule.

In another aspect, the invention provides a method of optically mapping DNA, wherein the method comprises providing a micropatterned substrate as described elsewhere herein; contacting the micropatterned substrate with a solution comprising at least one molecule of DNA, wherein one end of at least one molecule of DNA attaches to the binding region of the micropatterned substrate; and combing the at least one molecule of DNA such that the at least one molecule of DNA extends from the binding region into at least a portion of an adjacent non-binding region; and optically mapping the at least one molecule of DNA.

In yet another aspect, the invention provides a method of on surface DNA sequencing library generation, wherein the method comprises providing a micropatterned substrate, as described elsewhere herein; contacting the micropatterned substrate with a solution comprising at least one molecule of DNA, the at least one molecule of DNA comprising a T7 promoter; wherein one end of at least one molecule of DNA attaches to the binding region of the micropatterned substrate; combing the at least one molecule of DNA such that the at least one molecule of DNA extends from the binding region into at least a portion of an adjacent non-binding region; and generating a DNA sequencing library.

In yet another aspect, the invention comprises a method of DNA sequencing library generation, the method comprises providing a micropatterned substrate, as described elsewhere herein; contacting the micropatterned substrate with a solution comprising at least one molecule of DNA, the at least one molecule of DNA wherein one end of at least one molecule of DNA attaches to the binding region of the micropatterned substrate; combing the at least one molecule of DNA such that the at least one molecule of DNA extends from the binding region into at least a portion of an adjacent non-binding region; amplifying the at least one molecule of DNA using at least one isothermal amplification method, thereby forming an amplified product; eluting the amplified product from the device; and generating a DNA sequencing library using the eluted amplified product.

In yet another aspect, the invention provides a method of on surface DNA sequencing library generation, wherein the method comp comprises providing a micropatterned substrate, the micropatterned substrate, as described elsewhere herein; contacting the micropatterned substrate with a solution comprising at least one molecule of DNA, the at least one molecule of DNA wherein one end of at least one molecule of DNA attaches to the binding region of the micropatterned substrate; combing the at least one molecule of DNA such that the at least one molecule of DNA extends from the binding region into at least a portion of an adjacent non-binding region; performing a tagmentation reaction on the at least one molecule of DNA, thereby generating at least one tagmented product; amplifying the at least one tagmented product, thereby forming an amplified product; and generating a DNA sequencing library using the amplified product.

In yet another aspect, the invention provides a method of on surface DNA sequencing, wherein the method comprises: providing a micropatterned substrate, as described elsewhere herein; contacting the micropatterned substrate with a solution comprising at least one molecule of DNA, the at least one molecule of DNA wherein one end of at least one molecule of DNA attaches to the binding region of the micropatterned substrate; combing the at least one molecule of DNA such that the at least one molecule of DNA extends from the binding region into at least a portion of an adjacent non-binding region; and sequencing the at least one molecule of DNA.

In certain embodiments, the binding regions and the non-binding regions alternate across at least a portion of the substrate

In certain embodiments, the first width is 10 to 40 μm and the second width is 10 to 170 μm.

In certain embodiments, the combing comprises generating a receding meniscus.

In certain embodiments, the micropatterned substrate comprises a silica wafer.

In certain embodiments, the binding region comprises at least one selected from the group consisting of octenyl, octadecyl, docosenyl, SU-8, polymethylmethacrylate, polydimethylsiloxane, and polystyrene.

In certain embodiments, the non-binding region comprises at least one selected from the group consisting of polyethylene glycol (PEG), polyvinylpyrrolidone, and their derivatives.

In certain embodiments, the methods described herein further comprise coating the micropatterned substrate with a hydrogel.

In certain embodiments, the optical mapping of the at least one molecule of DNA comprises contacting the at least one molecule of DNA with at least one nicking endonuclease; incorporating at least one fluorescent dye-terminator into the at least one molecule of DNA; staining the at least one molecule of DNA; and imaging the at least one molecule of DNA.

In certain embodiments, the nicking endonuclease is selected from the group consisting of Nt.BspQI, Nb.BbvCI, Nt.BbvCI, Nb.BssSI, Cas9 nickase

In certain embodiments, optically mapping the at least one molecule of DNA comprises contacting the at least one molecule of DNA with at least one guide RNA sequence complementary to at least a portion of the at least one molecule of DNA and an inactive CRISPR-Cas9; and imaging the at least one molecule of DNA.

In certain embodiments, the imaging comprises fluorescence microscopy. In certain embodiments, the imaging comprises epifluorescence or total internal reflection fluorescence microscopy (TIRF).

In certain embodiments, the isothermal amplification method is selected from the group consisting of strand displacement at nicks or PNA-displaced sites.

In certain embodiments, sequencing the at least one molecule of DNA comprises one or more techniques selected from group consisting of: direct DNA sequencing by DNA polymerase with reversible DNA terminators; generating RNA from the at least one molecule of DNA using RNA polymerase and sequencing using T7 reverse transcriptase with reversible RNA terminators; amplifying the at least one DNA molecule on the substrate, and sequencing with reversible DNA terminators, by DNA ligation reaction with DNA ligase; and sequencing-by-hybridization using fluorescently labeled short oligonucleotides.

In certain embodiments, the method is performed in a flow cell.

In yet another aspect, the invention provides a method for mapping a genome, wherein the method is capable of resolving a single nucleotide polymorphism (SNP), the method comprising introducing to the genome a CRISPR/Cas9 system comprising at least one single-guide RNA (sgRNA) specific for a target sequence or a plurality of target sequences across the genome and a Cas9 D10A, wherein the CRISPR/Cas9 system nick labels the target sequence, and the target sequence or genome is analyzed.

### BRIEF DESCRIPTION OF THE DRAWINGS

The following detailed description of preferred embodiments of the invention will be better understood when read in conjunction with the appended drawings. For the purpose of illustrating the invention, there are shown in the drawings embodiments which are presently preferred. It should be understood, however, that the invention is not limited to the precise arrangements and instrumentalities of the embodiments shown in the drawings.

FIGS. 1A-1B illustrate micropatterning & dual-functionalizing glass substrates for DNA linearization. FIG. 1A illustrates fabrication process flow employed in micropatterning glass substrates; octenyl sections are 18 mm long and 10 to 40 μm wide; PEG sections are 10 to 170 μm wide. FIG. 1B shows individual DNA molecules that selectively end-adsorb to octenyl sections are subsequently linearized through traditional molecular combing with a receding meniscus. Molecules linearize across passivated, PEG sections, after which chemical modification/detection and visualization may be carried out.

FIGS. 2A-2C illustrates combing λ-DNA on micropatterned, dual-functionalized glass. λ-DNA molecules were combed onto 10-15 (FIG. 2A) and 10-40 (FIG. 2B) substrates. Octenyl sections appear as bright strips due to adsorption of YOYO-1 dye in contrast to the low-fluorescence background of PEG sections. Molecules were predominantly end-bound to the octenyl sections and extended across the PEG sections. Both, 10-15 and 10-40 resulted in similar linearization, with relatively lower DNA density on 10-40. Scale bars=10 μm. FIG. 2C shows overlaid histograms with matching Gaussian regressions demonstrate the length distributions of λ-DNA molecules combed on unpatterned octenyl substrate (blue, n=191) and micropatterned OTMS-PEG (red, n=116). OTMS-PEG substrate produced significantly lower DNA extension.

FIGS. 3A-3C illustrates combing hgDNA with various patterns. High molecular weight human DNA was combed on micropatterned OTMS-PEG substrates to demonstrate the surface's ability to adsorb and isolate long molecules and to explore the significance of pattern design parameters in combing long molecules. Human DNA combed on two patterns are shown here: 10-40 (FIG. 3A) and 40-170 (FIG. 3B). As in FIG. 2, molecules were seen to adsorb to the octenyl sections in an end-selective fashion; FIG. 3C: An example image of human DNA >2 Mbp long combed onto a 10-40 substrate (scale bar=50 μm).

FIGS. 4A-4C illustrates characterization of low-fluorescence background; on-surface nick labeling of hgDNA; on-surface transcription on T7 DNA. FIG. 4A is a magnified image of an octenyl section and adjoining PEG sections (10-40) shows suppressed binding of ATTO-532-dUTP (150 nM) to PEG compared to octenyl. FIG. 4B shows that ATTO-532-dUTP was successfully incorporated into combed hgDNA molecules using nick-labeling chemistry. FIG. 4C shows the transcription reaction performed using T7 RNAP on combed T7 DNA resulted in bright, labeled RNA aggregates along the T7 backbone.

FIG. 5A shows on-surface optical mapping of λ-DNA. FIG. 5A: top, i: BbvCI site distribution on λ-DNA; bottom, ii: corresponding simulated nick-label distribution on λ-DNA. FIGS. 5B-5D are microscope images of on-surface nick-labeled λ-DNA molecules that were concatemerized, combed, labeled, and stained. The thin arrows point to λ-DNA molecules that contain the 4 BbvCI nick-labels and the thick arrows point to partially and/or weakly labeled molecules. FIG. 5E: is a histogram showing the predicted BbvCI nick-label positions on λ-DNA backbone. The predicted positions were found to be 12, 17.3, 29.9, and 39.9 kbp corresponding to the actual averaged-out label positions, 12.7, 17.1, 30.2, and 40.5 kbp, respectively.

FIG. 6 depicts an embodiment of the substrate mounted on a microscope stage.

FIG. 7 depicts reference maps for ALU-1 (bottom) and 22qWhole (top).

FIG. 8 depicts 22q-Whole labeling of M14 DNA.

FIG. 9 depicts ALU-1 labeling on M14 DNA.

FIG. 10 illustrates interrogation of individual bases with CRISPR-Cas9 labeling. the thin horizontal lines indicate single molecules. The thick bars represent Nt.BSPQI reference map. The narrower bar represent consensus map of combined Nt.BSPQI CRISPR-Cas9 labeling. Arrows and bases indicate the single base differences between the two strains.

FIG. 11 illustrates the workflow of sgRNA synthesis. The multiple oligos with a promoter sequence and an overlap sequence on either side of the target sequence are hybridized with a single complementary oligo that shares the overlap sequence. The following exemplary sequences are shown: Step 1—top sequence (SEQ ID NO: 449) comprising T7 promoter (SEQ ID NO: 446), target sequence (SEQ ID NO: 450), and overlap sequence (SEQ ID NO: 447), and bottom sequence first region (SEQ ID NO: 451) and bottom sequence second region (SEQ ID NO: 452) comprising overlap sequence complement (SEQ ID NO: 453); Step 2—extended top sequence (SEQ ID NO: 454) comprising first extension region (SEQ ID NO: 455) and the top sequence second extension region (SEQ ID NO: 456) and the extended bottom sequence (SEQ ID NO: 457) comprising extension region (SEQ ID NO: 458); Step 3—first region of the transcript (SEQ ID NO: 459) and second region of the transcript (SEQ ID NO: 460); and Step 4—final sgRNA comprising a target sequence (SEQ ID NO: 461).

FIG. 12A illustrates mapping results of RR722 molecules labeled with the 48 sgRNAs (Table 2). The lines in the bar (designed reference map of RR722) represent the locations of the 48 sgRNAs on RR722. The thin lines below the reference are labels with dark dots representing where labels matched to the reference and light dots representing labels not found in the reference.

FIG. 12B illustrates mapping results of RR3131 molecules labeled with the set of 48 sgRNAs (Table 2). The lines in the bar (designed reference map of RR3131) represent the locations of the 48 sgRNAs on RR3131. The thin lines below the reference are labels with dark dots representing where labels matched to the reference map and light dots representing labels not found in the reference map. The red arrows indicate the off-target labeling.

FIG. 13 illustrates sgRNA design flow-chart

FIGS. 14A-14B illustrates mapping results of RR722 molecules labeled with the 162 sgRNAs (Table 5). In FIG. 14A, the lines in the bar (designed reference map of RR722) represent the locations of the 162 sgRNAs on RR722. The thin lines below the reference are labels with dark dots representing where labels matched to the reference and light dots representing labels not found in the reference. FIG. 14B shows the alignment results to RR3131.

FIG. 15A is an illustration of sequencing performed at multiple loci along single long DNA molecules for performing base-by-base sequencing for 10 bp at specific loci on single DNA molecules. Exemplary sequences are shown for 10 loci (SEQ ID NOs: 462-471).

FIG. 15B illustrates sequencing by synthesis using reversible terminator nucleotides.

FIG. 16 is schematic showing CRISPR-Cas9 DNA labeling.

FIG. 17A illustrates multi-color Cas9 nick-labeling; the 1st sgRNA probe will ‘map’ out DNA 2nd sgRNA probe can pinpoint variants.

FIG. 17B illustrates dCas9-based cyclic chemistry. This is association based chemistry and is single-step, faster, more gentle. With this studying binding dynamics is potentially possible.

FIG. 17C are images from dCas9-based cyclic chemistry, wherein reading 20 bases/cycle/site is possible.

FIG. 18 shows results from resolving a highly-conserved region between two H. influenzae strains with sequential labeling steps.

FIG. 19 depicts cycles of sequencing by cyclic dcas9-sgRNA binding using multiple fluorescent probes.

### DETAILED DESCRIPTION

Described herein is a microfabricated surface that can not only comb the DNA molecules efficiently but also provides for sequence-specific enzymatic fluorescent DNA labelling. By modifying a glass surface with two contrasting functionalities, such that DNA binds selectively to one of the two regions, DNA extension can be controlled, which is known to be critical for sequence-recognition by an enzyme. Moreover, the surface modification provides enzymatic access to the DNA backbone, as well as minimizing nonspecific fluorescent dye adsorption. These enhancements make the designed surface suitable for largescale and high-resolution single DNA molecule studies.

### Definitions

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein. In describing and claiming the present invention, the following terminology will be used.

It is also to be understood that the terminology used herein is for the purpose of describing particular embodiments only, and is not intended to be limiting.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass non-limiting variations of ±20% or ±10%, ±5%, ±1%, or ±0.1% from the specified value, as such variations are appropriate.

As used herein, the term “inactive CRISPR-Cas9” or “dCas9” means a mutant Cas9 enzyme that is devoid of endonuclease activity, limiting its function to programmable RNA-guided sequence-specific binding to DNA.

As used herein, the term “fluorescence microscopy” means optical microscopy that employs the phenomenon of fluorescence to form an image of the object. The fluorescing object is excited by light of higher wavelength, and the emitted light of lower wavelength is collected to form an image.

The term “total internal reflection fluorescence microscopy” or “TIRF” is a fluorescence microscopy technique consisting a special illumination technique to generate evanescent light waves at the fluorescent sample interface. This results in high axial resolution, usually 200 nm or less, suitable to screen out high fluorescence background.

The term “fluorescent dye-terminator” means a fluorophore-tagged reversible-terminating nucleotide. A reversible-terminating nucleotide or a reversible terminator is a modified deoxynucleotide analog that reversibly terminates primer extension by a polymerase. Upon mild chemical treatment or photocleavage, the termination function in reversed and primer extension may resume.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Description

### Methods of Immobilizing and Linearizing Oligonucleotides on a Micropatterned Substrate

In one aspect, the invention provides a method of immobilizing and linearizing an oligonucleotide, the method comprising providing a micropatterned substrate, the micropatterned substrate comprising at least one binding region having a first width and at least one non-binding region having a second width; wherein the binding regions and non-binding regions alternate across at least a portion of the substrate; contacting the micropatterned substrate with a solution comprising a plurality of oligonucleotides, wherein one end of at least one oligonucleotide molecule attaches to a binding region of the micropatterned substrate; and combing the at least one oligonucleotide molecule such that the at least one oligonucleotide molecule extends from the binding region into at least a portion of an adjacent non-binding region; thereby immobilizing and linearizing the at least one oligonucleotide molecule.

In various embodiments, the first width is about 10 to about 40 μm and the second width is about 10 to about 170 μm. In various embodiments, the first width is about 10 μm and the second width is about 40 μm. In various embodiments, the first width is about 10 μm and the second width is about 15 μm. In various embodiments, the first width is about 10 μm and the second width is about 170 μm.

The materials from which the micropatterned substrate is made are not particularly limited. A person of ordinary skill in the art in possession of this disclosure is able to select an appropriate substrate onto which the binding and non-binding regions are placed. In various embodiments, the micropatterned substrate comprises a silica or a silicon wafer.

In various embodiments, the binding region comprises a material to which DNA and other oligonucleotides attach with high affinity. The attachment may be covalent or non-covalent. In various embodiments, the binding region comprises at least one selected from the group consisting of octenyl, octadecyl, docosenyl. In various embodiments the binding region comprises octenyl. In various embodiments the binding region comprises a hydrophobic polymer coating. In various embodiments the hydrophobic polymer coating is selected from the group consisting of SU-8, polymethylmethacrylate, polydimethylsiloxane, polystyrene. Any long-chain aliphatic functional group such as hexyl, undecyl, (or their vinyl-terminated derivatives-hexenyl, undecenyl) are known to immobilize DNA molecules and therefore may be used as hydrophobic polymers to form the binding region in various embodiments of the invention. Multiple hydrophobic polymers are also known to do the same. In various embodiments, the hydrophobic polymer is selected from the group consisting of cyclicolefin copolymers, polydimethylsiloxane, poly(methyl methacrylate) and polystyrene.

In various embodiments, the non-binding comprises a material to which DNA and other oligonucleotides attach do not attach or do not attach with high affinity. In various embodiments, the non-binding region comprises at least one selected from the group consisting of polyethylene glycol (PEG) and polyvinylpyrrolidone. In various embodiments the non-binding region comprises PEG or a PEG derivative including but not limited to Tween, e.g. Tween-20, or Triton X-100.

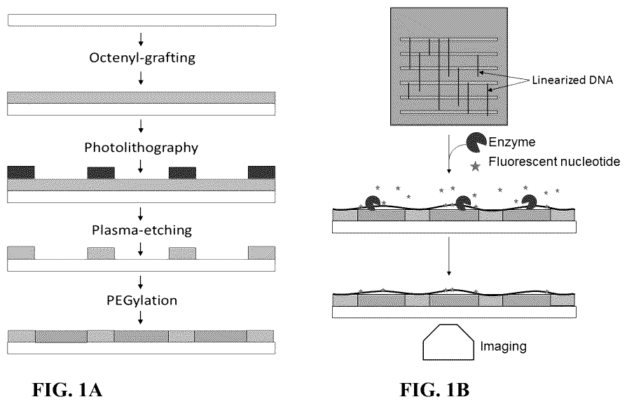

One example of a method for producing the micropatterned substrate is illustrated in FIG. 1A and discussed further herein under Materials and Methods. In various embodiments, providing the micropatterned substrate comprises manufacturing the substrate by any means known in the art. In other embodiments, providing the substrate comprises placing the micropatterned substrate in position to begin the method, e.g. in a flow cell, on a microscope stage, etc.

Various techniques for DNA combing are known in the art and all of them are contemplated in combination with the present invention. In various embodiments, the combing comprises generating a receding meniscus.

In various embodiments, the method further comprise coating the micropatterned substrate with a hydrogel after the DNA combing step is performed. In various embodiments, the hydrogel comprises polyacrylamide. In various embodiments, the hydrogel comprises agarose, paraformaldehyde or PEG-acrylate.

In various embodiments of this aspect and the aspects described below, the method is performed in a flow cell. Various configurations of flow cell are available and can be selected by a person of ordinary skill in the art.

### Methods of Optically Mapping Immobilized and Linearized DNA on a Micropatterned Substrate

In one aspect, the invention provides a method of optically mapping DNA, the method comprising: providing a micropatterned substrate, the micropatterned substrate comprising: at least one binding region having a first width; and at least one non-binding region having a second width; wherein the binding regions and the non-binding regions alternate across at least a portion of the substrate; contacting the micropatterned substrate with a solution comprising at least one molecule of DNA, wherein one end of at least one molecule of DNA attaches to the binding region of the micropatterned substrate; and combing the at least one molecule of DNA such that the at least one molecule of DNA extends from the binding region into at least a portion of an adjacent non-binding region; and optically mapping the at least one molecule of DNA.

Various methods of optically mapping DNA are known to one of ordinary skill in the art and all are contemplated for use in combination with the present invention. In various embodiments, optical mapping of DNA is performed by using nicking endocnucleases and DNA polymerase to insert various fluorescent dye-terminators into the molecule or molecules of DNA under interrogation. In various embodiments, optically mapping the at least one molecule of DNA comprises contacting the at least one molecule of DNA with at least one nicking endonuclease; incorporating at least one fluorescent dye-terminator into the at least one molecule of DNA; staining the at least one molecule of DNA; and imaging the at least one molecule of DNA.

In various embodiments, various nicking endonucleases are employed depending on the sequence of the DNA molecule under interrogation. In various embodiments, the nicking endonuclease is selected from the group consisting of Nt.BspQI, Nb.BbvC1, Cas9 nickase, Nb.BssSI.

In various embodiments, incorporating fluorescent dye-terminators into is performed by contacting the at least one DNA molecule with a solution comprising one or more fluorescent dye terminators and at least one DNA polymerase. A person of skill in the art is able to select a suitable polymerase based on the specifics of the method as described herein.

In various embodiments, optically mapping comprises contacting the DNA with a solution comprising inactive CRISPR-Cas9 (dCas9) and a suitable guide RNA based on the sequence of the DNA to be interrogated such that the guide RNA/dCas9 complex binds to the DNA. The bound complex is then detected. In various embodiments, optically mapping the at least one molecule of DNA comprises contacting the at least one molecule of DNA with at least one guide RNA sequence complementary to at least a portion of the at least one molecule of DNA and an inactive CRISPR-Cas9; and imaging the at least one molecule of DNA.

In various embodiments, imaging comprises any technique that allows the detection and location of the labeled DNA molecules. In various embodiments, imaging comprises fluorescence microscopy. In various embodiments, imaging comprises epifluorescence or total internal reflection fluorescence microscopy (TIRF). In various embodiments, the method further comprises various steps of data processing to interpret data obtained during the imaging step. Various software is available commercially and a person of ordinary skill in possession of this disclosure is able to select a suitable technique from the relevant literature or to generate their own methodology.

### Methods of on Surface DNA Sequencing Library Generation

In another aspect, the invention provides a method of on-surface DNA sequencing library generation, the method comprising immobilizing and linearizing DNA as described above, and contacting the at least one molecule of DNA with at least one RNA polymerase, thereby generating at least one molecule of RNA. Following RNA generation, the library may be generated by contacting the at least one molecule of RNA with at least one reverse transcriptase, thereby converting the at least one molecule of RNA to cDNA, followed by eluting the cDNA from the device. The eluted cDNA is used to generate a DNA sequencing library. In some aspects, the at least one molecule of DNA comprises a T7 promoter to facilitate RNA generation.

In another aspect, the invention provides a method of on-surface DNA sequencing library generation, the method comprising immobilizing and linearizing DNA as described above, and amplifying the at least one molecule of DNA using at least one isothermal amplification method, thereby forming an amplified product; and eluting the amplified product from the device. The eluted amplified product is converted to a DNA sequencing library. In various embodiments, the isothermal amplification method is selected from the group consisting of strand displacement at nicks or PNA-displaced sites.

In another aspect, the invention provides a method of on-surface DNA sequencing library generation, the method comprising immobilizing and linearizing the DNA as described above and performing a tagmentation reaction on the at least one molecule of DNA, thereby generating at least one tagmented product; amplifying the at least one tagmented product, thereby forming an amplified product. The amplified product is eluted from the device and is used to generate a DNA sequencing library. In various embodiments, the DNA sequencing library is generated by contacting the amplified product with at least one RNA polymerase, thereby generating at least one molecule of RNA; and contacting the at least one molecule of RNA with at least one reverse transcriptase, thereby converting the at least one molecule of RNA to cDNA and generating a DNA sequencing library.

The methods of generating DNA sequencing libraries described herein may be directed to the entire genome or to targeted regions. In various embodiments the DNA molecules are chosen based on target specific labeling using a CRISPR-Cas9 labeling system before performing the above steps.

### Methods of on Surface DNA Sequencing

In another aspect, the invention provides a method of on-surface DNA sequencing, the method comprising immobilizing and linearizing DNA as described and sequencing the at least one molecule of DNA. In various embodiments, sequencing the at least one molecule of DNA comprises one or more techniques selected from group consisting of direct DNA sequencing by DNA polymerase with reversible DNA terminators; generating RNA from the at least one molecule of DNA using RNA polymerase and sequencing using T7 reverse transcriptase with reversible RNA terminators; amplifying the at least one DNA molecule on the substrate, and sequencing with reversible DNA terminators, by DNA ligation reaction with DNA ligase; and sequencing-by-hybridization using fluorescently labeled short oligonucleotides.

### Method for Mapping a Genome, Wherein the Method is Capable of Resolving a Single Nucleotide Polymorphism (SNP)

In yet another aspect, the invention provides a method for mapping a genome, wherein the method is capable of resolving a single nucleotide polymorphism (SNP), the method comprising introducing to the genome a CRISPR/Cas9 system comprising at least one single-guide RNA (sgRNA) specific for a target sequence or a plurality of target sequences across the genome and a Cas9 D10A, wherein the CRISPR/Cas9 system nick labels the target sequence, and the target sequence or genome is analyzed.

In certain embodiments, the analyzing is by nucleotide sequencing and/or imaging.

In certain embodiment, the genome is a human genome or a microbial genome. In certain embodiments, the method is capable of distinguishing a microbe from another closely-related microbe.

In certain embodiments, the SNP is in a protospacer adjacent motif (PAM SNP) sequence. In certain embodiments, the at least one sgRNA targets a PAM and/or a PAM SNP.

In certain embodiments, the method is capable of mapping a genomic region that spans a length of at least 1 kb, 10 kb, 100 kb, 300 kb, or 500 kb in the genome.

In yet another aspect, the invention provides a method of defining a long distance haplotype in a genome, the method comprising administering to the genome a CRISPR/Cas9 system comprising a Cas9 D10A and a plurality of single-guide RNAs (sgRNAs) specific for a plurality of loci of a genomic region or a plurality of target regions across the genome, wherein the CRISPR/Cas9 system nick labels the plurality of loci of the genomic region or the plurality of target regions across the genome, and the target sequence or genome is analyzed thereby defining the long distance haplotype in the genome.

In certain embodiments, the genome is a human genome or a microbial genome.

In certain embodiments, the plurality of sgRNA comprises at least one sgRNA that targets a PAM or a PAM SNP.

In yet another aspect, the invention provides a method for customized mapping of a whole genome, the method comprising, nick labeling the genome with a CRISPR/Cas9 system and analyzing the nucleotide sequence, wherein the CRISPR/Cas9 system comprises a Cas9 D10A and a plurality of sgRNAs designed by a method comprising:
a) performing in silico analysis to predict sgRNAs sequences that comprise a single perfect match to the genome,
b) retaining from step a) all sgRNA sequences that contain a single perfect match to the genome within the 8-based seeding sequence proximal to the PAM,
c) retaining from step b) all sgRNA sequences will less than 5 total single base mismatches in the proximal 8 bp of the genome,
d) retaining from step c) sgRNA sequences with less than 5 total single base mismatches in the distal 12 bp of the genome.

In certain embodiments, the microbe is distinguished at the strain level.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore, specifically point out the preferred embodiments of the present invention, and are not to be construed as limiting in any way the remainder of the disclosure.

### Materials and Methods

### Glass Surface Functionalization

Glass coverslips (22×22 mm, VWR 48366-067) were used as substrates to covalently graft octenyl, PEG, and 1-amino-undecane (AU) functional groups via silanization reaction with 7-octenyltrimethoxysilane (OTMS) (Gelest, SIO6709.0), 2-[methoxy (polyethyleneoxy) 6-9 propyl] trimethoxysilane (PTMS) (Gelest, SIM6492.7), and 11-aminoundecyltriethoxysilane (AUTS) (Gelest, SIA0630.0) respectively. Briefly, surface groups of cleaned substrates were activated by treatment with either highly corrosive “piranha” solution or air plasma etching (Femto science, CUTE, 200W 1-3 min). Activation exposed silanol groups on the glass surface, and under low-humidity conditions (<10% RH) reacted with the silane solution producing clear coatings of the respective functional groups. Reaction temperatures were between 21 and 23° C.

### Micropatterning Surface Functionalization

Micropatterning was performed in a class 10,000 cleanroom using positive photolithography. The fabrication process flow is shown in FIG. 1A.The octenyl-functionalized surface was coated with the positive PR (Microposit SC1813 or SC1827; Down Corning), aligned underneath a photomask with the desired pattern, and exposed to UV light. The substrates were then developed using Microposit 351, dried using nitrogen, and loaded into the air-plasma etcher. Octenyl coating in the exposed regions on the substrate was etched away and the underlying glass was re-activated with silanol groups. Micropatterned substrates were loaded onto polypropylene coverslip racks, and PR was stripped off the surfaces by sequential washing in acetone-isopropylalcohol-water held inside an ultrasonic bath (Branson 2510). After this, substrates were dried with filtered nitrogen gas and transferred to Columbia jars (Wheaton). Freshly prepared PTMS solution in toluene was added to the jars and sealed under desiccating atmosphere.

Photomasks were designed using a CAD program and ordered from CAD/Art Services, Inc (Bandon, OR). A single pattern contained repetitive regions of inked and transparent bands with definite line widths and spacing. For example, one pattern consisted of 10 um-wide inked lines with 40 um-spacing, that we term ‘10-40’. Similarly, 10-10, 10-15, 20-90 and 40-170 patterns were also designed. The objective was to maximize the area of PEG region containing combed DNA for fluorescence visualization, without any loss in DNA combing density.

### High Molecular Weight DNA Extraction

Mammalian cells were embedded in gel plugs and High Molecular Weight DNA was purified as described in a commercial large DNA purification kit (BioRad #170-3592). Plugs were incubated with lysis buffer and proteinase K for four hours at 50° C. The plugs were washed and then solubilized with GELase (Epicentre). The purified DNA was subjected to 2.5 hours of drop-dialysis. It was quantified using Quant-iT dsDNA Assay Kit (Life Technology), and the quality was assessed using pulsed-field gel electrophoresis.

### DNA Linearization by Molecular Combing

Briefly, DNA samples were prepared for molecular combing in 50 mM MES, 100 mM NaCl, pH 5.5-6.0 at concentrations ranging from 0.1 to 0.6 ng/μL. The substrate was first immersed into DNA solution for a two-to-twenty-minute dwell time to allow the partially denatured tail ends to interact with the substrate. It was then withdrawn at a rate of 100 μm/s using a translational stage (Thorlabs MTS25-Z8).

### Flow-Forced DNA Linearization

An SU-8 mold with channel widths ranging from 1 to 18 mm and heights ranging from 10 to 180 μm was fabricated. After casting PDMS, individual channels were cut out and fluid ports were bored with a biopsy punch. The face of the imprinted PDMS block was then air plasma treated and adhered to the functionalized substrate to create a liquid-tight flow cell. DNA was adsorbed and linearized using flow cells. Briefly, 2-4 μL of YOYO-1-stained (100 nM) λ bacteriophage DNA in TE buffer (pH 8.0) was added into the flow cell port. The shear force exerted by the flowing buffer solution linearized the DNA as it adsorbed onto the positively-charged AU surface.

### Hydrogel Layer Preparation and Assembly

Polyacrylamide gel was used to maintain a stable aqueous environment around the DNA backbone. After combing the DNA onto micropatterned substrate, a low-adhesion PVC tape (18733, Semiconductor Equipment Corp) that was cut to specific dimensions (as that of the desired ‘microliter-well’) was transferred onto the micropatterned substrate. This tape acted as a stencil delimiting the casting area of polyacrylamide gel. Polyacrylamide gel was prepared (4-10%) and pipetted at one-end of the microliter-well. A glass slide that was coated with the PVC tape was used to spread the gel droplet throughout the stenciled microliter-well area. After 5 mins of casting time, the slide and micropatterned substrate are gently separated from each other. The polyacrylamide layer is then hydrated immediately with CutSmart 1×buffer, before preparing for the next step in device assembly.

### Device:

Polyacrylamide gel overlay: The linearized DNA is susceptible to damage under the effect of flow forces. A polyacrylamide gel overlay helps prevent this damage. But addition of a gel layer would impede diffusion kinetics of the reagents, unless it is made as a thin film with a thickness 10 μm or below. Reaction times with the current prototype, that uses 75 μm gel, are in the range of 1-1.5 h—this will be reduced to <1 min if a 1 μm-thick gel overlay is used. However, fabricating films of such low thickness was challenging, possible due to insufficient diffusion during gel polymerization. We devised a way to fabricate thin polyacrylamide gel films by the addition of methacrylate functional group (or equivalent) to the PEG sections of the device so as to seed gel formation. Addition of a participating chemical group to the surface has resulted in films of lower thicknesses than without the participating group.

Polyacrylamide gel casting device: The gel was cast by using a spacer whose height can be controlled. A specially designed device was constructed to enable thin film fabrication on the micropatterned substrate. This device consists of a PDMS-coated glass slide, defined photoresist spacer films, and inlet and outlet ports for addition of the pre-polymer gel mixture. PDMS was coated on a glass slide to form a strong, durable hydrophobic coating. We have used SU-8 photoresist to form the spacer and defined its height by the viscosity and spin speed during its coating on the PDMS-coated glass slide. PDMS, being too hydrophobic for SU-8 spread, breaks the SU-8 film after spin coating. For this, we optimized an SU-8 coating protocol with extended soft-bake times (5-15 min) on hotplate at lower temperatures (than the recommended 95° C.), followed by soft-bake in a gravity oven (15-20 min) at 95° C.

Temperature and microfluidics control: The gel-overlaid micropatterned substrate is mated with an optimized microfluidic channel array, made of a machinable polymer such as PMMA, PDMS, and others. The assembled device is placed in a compact heat control instrument that uses a thermoelectric element to maintain optimal reaction temperature throughout the sequencing reaction. The heat control instrument would be capable of maintaining reaction temperatures in the range of 37-65° C. The primary performance aspect for the instrument is temperature stability. Using temperature probes local to the reaction volume, we will optimize the control parameters. In a variation, these temperature probes may be embedded into the microchannel array to provide a closed-loop control.

### Microliter-Well Assembly for On-Surface Reactions

Enzymatic reactions were performed in two formats: (1) PDMS reaction wells assembled atop micropatterned substrate, and (2) PDMS-PMMA composite assembly on top of the substrate with a cast PA gel.

PDMS slabs, that were cast in plastic dishes, were cut into approximately 12×20 mm blocks. PDMS was adhered to the functionalized substrate by either double-sided tape or plasma activation. PDMS adhered using double sided tape was first mated to a strip of double-sided tape and then an array of reaction wells was created using a 4 mm biopsy punch. PDMS adhered with plasma activation first had an array of wells punched out, followed by a 2-minute plasma treatment (Harrick Plasma, PDC-32G). DNA was combed onto functionalized substrates, allowed to dry at room temperature for 5 minutes, and the prepared PDMS well blocks were carefully positioned onto the targeted combing region. Each well was used for a unique experimental reaction condition. This microwell-format was used for reaction without a protecting hydrogel layer.

A PMMA sheet was laser cut to form the top and bottom layers of the device assembly, as well as to generate molds for PDMS gaskets that will surround the gel region of the microwell-plate. PDMS was cast into these molds and the resulting gaskets were mated to the PMMA top layer and placed over the gel-coated substrate such that the gaskets surrounded the gel area without any contact. This assembly was then clamped to the PMMA bottom layer. The mouths of the microliter-wells are sealed with a tape, creating a tightly-sealed compartment for carrying out reactions.

### On-Surface Transcription on T7 DNA

T7 phage DNA (500 ng) was added into a combing reservoir containing 50 mM MES, 100 mM NaCl, pH 6.0 buffer and homogenized for 1 hour before combing onto 10-10 and 10-15 micropatterned substrates. Reaction wells were assembles as described above. Combed DNA molecules were rehydrated with rehydration buffer (0.1% BSA, 20 μM NTPs, 1 mM DTT, 5 mM MgCl₂, 50 mM Tris, pH 7.8) for 2 minutes. T7 RNA polymerase (RNAP) reaction buffer from New England Biolabs diluted to 1×concentration (40 mM Tris-HCl, 5 mM MgCl₂, 1 mM DTT, pH 7.8) was then added to prime the same well for an additional minute. The master mix for transcription reaction is prepared in a 0.6 ml microcentrifuge tube prior to pipetting into the well. Reaction mix contains 2.5 U of T7 RNAP, 10 μM Cy3-UTP, 200 μM NTPs, 100 μM DTT, 1 U/μL RiboGuard RNase inhibitor (Lucigen), 1×T7 RNAP reaction buffer. The mixture was gently pipetted into the well and the device was incubated in a humidified oven at 37° C. for 1 h. The well was evacuated and washed with 1×RNAP reaction buffer. The DNA backbone was stained with YOYO-1.

### On-Surface Nick-Labeling of Combed hgDNA

Human DNA (500 ng) was suspended in a combing reservoir containing 50 mM MES, 100 mM NaCl, pH 6.0 buffer and let homogenize overnight before combing onto micropatterned substrates. After the assembly of PDMS reaction wells, combed DNA molecules were rehydrated with NEB 3.1 buffer for up to 15 minutes and then evacuated. Nt.BspQI (5 U) diluted in NEBuffer 3 (New England Biolabs) was added to the reaction well and incubated at 37° C. in a humidified oven for an hour. This will create the nicking sites for polymerase extension. The reaction mix was now evacuated and washed twice with NEBuffer 2.0, following which up to 5 U of either Taq DNA polymerase or DNA polymerase I (New England Biolabs) and dye-nucleotide mix (25-133 nM each of ATTO-532-dUTP, dATP, dGTP, gCTP) were added and let incubate at 37° C. for an hour inside a humidified oven to incorporate fluorescent dUTPs. After washing away the free dyes, the DNA backbone is subsequently stained with (YOYO-1) iodide (Life Technologies, Y3601). For some observations, labeled DNA was not stained before visualization on the microscope.

### On-Surface λ-DNA Mapping

To ensure observation of full-length λ-DNA molecules within the PEG section, DNA was concatemerized by heat-treating in 10 mM Tris-HCl buffer, pH 7.8, for 10 min at 65° C. followed by 1 h incubation at 37° C. After this, DNA was suspended in a reservoir for combing onto a 10-40 substrate. PA gel was cast onto the surface of two microliter-wells and a device was assembled as described earlier. A nicking mix with 20 U of Nb.BbvCI (New England Biolabs) in 1×CutSmart buffer (New England Biolabs) was added onto the gel surface of one of the microliter-wells. In the control well, 1×Cutsmart buffer was added. The device was incubated at 37° C. for 2 h, after which both wells were evacuated and washed with 1×CutSmart buffer. Next, a labeling mix with 10 U of Klenow Fragment (3′→5′ exo-) (New England Biolabs), ATTO-532-dUTP (266 nM), and dATP/dGTP/dCTP (each 133 nM) in NEBuffer 2 (New England Biolabs) was added to both the wells. The labeling reaction was performed at 37° C. for 2 h, following which the wells were evacuated and washed with 1×NEBuffer 2 thoroughly before imaging. After acquiring a few images, 100 nM YOYO-1 solution was added to the wells to stain the DNA backbone for re-imaging.

### Image Acquisition and Analysis

Imaging was performed on a custom-built, semi-automated inverted fluorescent microscopy system. It includes a Rapid Automated Modular Microscope and Modular Infinity Microscope system (ASI) with an XYZ motorized stage (ASI, MS-2000), CRISP autofocus system (ASI), and high-speed filter wheel (Finger Lakes Instrumentation, HS-625) combined with a 100× oil-immersion objective (Olympus, UPlanSApo, NA=1.40). Diode-pumped solid-state laser light sources with 473 nm and 532 nm wavelengths (LASEVER, LSR473ML-100, LSR532ML-200), controlled through u Manager (Open Imaging) using a custom-made TTL control system were used. Images were acquired with an iXon EMCCD (Andor, DU-888E-C00-#BV) or ORCA-Flash4.0 V2 CMOS (Hamamatsu, C11440).

Data collected from the imaging system is processed on a computing cluster in ImageJ using previously developed computational methods and algorithms together with manual curation. Images were first processed to remove background signal and normalize signal intensity. Once processed, images were analyzed semi-automatically using the Ridge Detection ImageJ plug-in.

### Sequencing

### The Sequencing Chemistry With Single Base Incorporation

a) Defined initialization points with Nickase and then followed by the polymerase cyclic incorporation of single reversible terminators.
b) Random initialization points with DNasI and then followed by the polymerase cyclic incorporation of single reversible terminators.
c) Defined initialization points with cas9-nickase-sgRNA and then followed by the polymerase cyclic incorporation of single reversible terminators.

### The Sequencing Chemistry With Ligation

a) Defined initialization points with Nickase and then followed by the ligase cyclic ligation of a short color-coded oligo
b) Random initialization points with DNasI and then followed by the ligase cyclic ligation of a short color-coded oligo
c) Defined initialization points with cas9-nickase-sgRNA and then followed by the ligase cyclic ligation of a short color-coded oligo
   The Sequencing Chemistry With Cyclic dcas9-sgRNA Binding
a) Barcode the sgRNA with multiple color fluorescent probes
b) The new ability of synthesis of 200 sgRNAs in a single tube reactions
c) In each cyclic reaction, multiple color-coded dcas9-sgRNAs will bind to multiple loci (20 bp of each locus) along the megabase long DNA molecules. Their precise locations will be imaged and recorded. The dcas9-sgRNAs will be then removed by protease and repeat the above process with a different set of dcas9-sgRNAs. Every cycle, we read multiple 20 bp sequences along the megabase long DNA molecules. The process will repeat many times on the same megabase long DNA molecules.
   The Sequencing Chemistry With Cyclic cas9-sgRNA Nick-Labeling
a) The new ability of synthesis of 200sgRNA in a single tube reactions
b) In each cyclic reaction, multiple cas9-nickase-sgRNAs will bind to multiple loci (20 bp of each locus) and create nicks along the megabase long DNA molecules. then followed polymerase incorporation of a fluorescent nucleotide. Their precise locations will be imaged and recorded. The fluoresnce dye will be bleached or removed. The above process will be repeated with a different set of cas9-nickase-sgRNAs. Every cycle, we read multiple 20 bp sequences along the megabase long DNA molecules. The process will repeat many times on the same megabase long DNA molecules.

### Flap Sequencing:

In this method, we create nicks in the linearized DNA either by use of an enzyme or using physical means such as heat. The created 3′ ends will be extended using a strand-displacing polymerase to generate flap strands. This generated single strand DNA flap would be sequenced using sequencing-by-ligation or sequencing by hybridization. In a variation, these flap sequences can be quickly detected using the robust Hybridization Chain Reaction, providing a simpler means to map DNA sequences.

Combinations: The above methods of manipulation may be combined and applied to the linearized DNA.

### High-Molecular-Weight DNA Extraction

Two Haemophilus influenzae strains with complete genome sequences were used: the standard lab strain Rd KW20 (RR722, NC_000907) and a marked derivative of clinical isolate 86-028NP (RR3131, NC_007416.2, carrying novobiocin and nalidixic acid resistance alleles, Nov^{R} and Nal^{R})(25,31,32). Bacterial culture followed standard protocols; cells were grown to stationary phase (OD₆₀₀ₙₘ=1.2) in supplemented brain-heart infusion (10 μg/ml hemin 2 μg/ml NAD) shaking at 37° C., and then cells were harvested by centrifugation at 4,000 rpm for 5 minutes before DNA extractions (33,34). Purification of ultra-high MW DNA fragments followed the Bionano Prep Cell Culture DNA Isolation Protocol. Briefly, cells were: (a) resuspended in cell buffer (˜5×10⁹ CFU/ml); (b) embedded in 2% low-melt agarose (BioRad) plugs to minimize shearing forces; (c) lysed using Bionano cell lysis buffer supplemented with 167 μl Proteinase K (Qiagen) rocking overnight at 50° C.; (d) RNase treatment by adding 50 μl of RNase A solution and incubating the plugs for 1 hour at 37° C. (Qiagen); and (c) washing in TE buffer with intermittent mixing. Finally, DNA was purified from low-melt agarose plugs by drop dialysis. Plugs were melted at 72° C., then incubated with 2 μl agarase (Thermo Fisher Scientific) for 45 minutes. Melted plugs were dialyzed into TE buffer using 0.1 μm Millipore membrane filters for 45 minutes at a ratio of 15 ml buffer per ˜200 μl sample. DNA was allowed to homogenize overnight at room temperature before fluorometric quantification using the Qbit dsDNA BR kit (Thermo Fisher Scientific).

### dsDNA Synthesis

sgRNA oligos: sgRNAs were encoded on 55 nt DNA oligos with a 5′ T7 promoter sequence (5′-TTCTAATACGACTCACTATAG-3′) (SEQ ID NO: 446), followed by the target 20mer sequence, complementary to the target gDNA sequence, and finally an overlap sequence (5′-GTTTTAGAGCTAGA-3′) (SEQ ID NO: 447). Individually synthesized sgRNA oligos were then pooled into an equimolar mixture. sgRNA complementary oligo: An 80 nt long oligo was designed with the 3′ end complementary to the overlap sequence and remainder encoded the Cas9 binding sequence (5′-AAAAGCACCGACTCGGTGCCACTTTTTCAAGTTGATAACGGACTAGCCTTATTTTAA CTTGCTATTTCTAGCTCTAAAAC-3′) (SEQ ID NO.448). All oligos are obtained from Integrated DNA Technology. The sgRNA oligo mix was hybridized to the sgRNA complementary oligo (at 10 μM each) in 1×NEBuffer2 (New England BioLabs, NEB) with 2 mM dNTPs at 90° C. for 15 sec followed by 43° C. for 5 min. To complete dsDNA synthesis, the hybridization mixture was incubated at 37° C. for 1 hr with 5 U of Klenow Fragment 3′→5′ exo-(NEB). To degrade linear ssDNA remaining, the dsDNA was then treated with Exonuclease I in 1×Exonuclease I reaction buffer (NEB) for 1 hr at 37° C. Finally, dsDNA was purified using QIAquick Nucleotide Removal Kit (Qiagen) and eluted in 30 ul elution buffer. Quality and concentration were assessed using agarose gel electrophoresis and the Synergy H1Hybrid Multi-Mode Reader (Bio Tek).

### sgRNA Synthesis

sgRNA was synthesized using HiScribe T7 High Yield RNA Synthesis Kit (NEB) following the Standard RNA Synthesis protocol. In summary, lug dsDNA was incubated with 1×reaction buffer, 10 mM NTPs and T7 RNA polymerase enzyme mix at 37° C. for 2 hrs followed by DNase I treatment at 37° C. for 15 min to remove dsDNA from the reaction. sgRNA was then purified using RNA Clean & Concentrator Kits (Zymo Research). The concentration of the purified sgRNA was assessed using Synergy H1Hybrid Multi-Mode Reader (Bio Tek).

### CRISPR-Cas9 Labeling of Chromosomal DNA

For DNA nicking using the 48 and 162 sgRNA mix (Table 3 and Table 4), 1.25 μM of the synthesized sgRNA was first incubated with 5 μM of Cas9 D10A (NEB) in 1×NEBuffer 3.1 (NEB) at 37° C. for 15 min to form a sgRNA-Cas9 complex. 300 ng of the DNA sample was then added to the sgRNA-Cas9 complex mixture and incubated at 37° C. for 60 min. For DNA nicking with both Cas9 and Nt.BspQI, 2.5 μM gRNA was first incubated with 100 ng of Cas9 D10A in 1× NEBuffer 3.1 at 37° C. for 15 min. After that, 300 ng of DNA and 5 U of Nt.BspQI (NEB) were added to the sample mixture and incubated at 37° C. for 2 hours. The nicked DNA samples were then labeled using 5 U Taq DNA Polymerase (NEB), 1×thermopol buffer (NEB), 266 nM free nucleotides mix (dATP, dCTP,dGTP (NEB) and Atto-532-dUTP (Jena Bioscience)) at 72° C. for 60 min. the labeled sample was then treated with Proteinase K at 56° C. for 30 min and 1 μM IrysPrep stop solution (BioNano Genomics) was added to the reaction.

### DNA Loading and Imaging

Labeled DNA samples were stained and prepared for loading on an Irys Chip (BioNano Genomics) following manufacturer instructions. The sample was then linearized and imaged. The stained samples were loaded and imaged inside the nanochannels following the established protocol. Each Irys Chip contains two nanochannel devices, which can generate data from >60 Gb of long chromosomal DNA fragments (>150 kb). The image analysis was done using BioNano Genomics commercial software (Irys View 2.5) for segmenting and detecting DNA backbone YOYO-1 staining, similar to early optical mapping methods, and localizing the green labels by fitting the point-spread functions.

### Data Analysis

Briefly, the assembler is a custom implementation of the overlap-layout-consensus paradigm with a maximum likelihood model. An overlap graph was generated based on the pairwise comparison of all molecules as input. Redundant and spurious edges were removed. The assembler outputs the longest path in the graph and consensus maps were derived. Consensus maps are further refined by mapping single-molecule maps to the consensus maps and label positions are recalculated. Refined consensus maps are extended by mapping single molecules to the ends of the consensus and calculating label positions beyond the initial maps. After the merging of overlapping maps, a final set of consensus maps was output and used for subsequent analysis. RefAligner works similarly but compares molecules directly to an in silico nicked reference instead of first forming contigs. These maps were then opened in Irsyview visualization software from BioNano Genomics.

The results of the experiments are now described.

### EXAMPLE 1

The micropatterned surface is dual-functionalized with two repetitive functional areas. One area is functionalized with octenyl, which is hydrophobic and adsorbs the tail-ends of DNA molecules. The other area is functionalized with polyethylene glycol (PEG), a passivating group which does not attract DNA and prevents the attachment of free stain and labeled nucleotide molecules. With this micropatterned surface, DNA molecules bind in an end-selective manner to the hydrophobic octenyl surface only, and then linearize uniformally through PEG regions by receding meniscus through dynamic combing. DNA molecules can be stretched in an orderly fashion with less potential for formation of both intermolecular intersections and intramolecular loops.

### DNA Adsorption on Octenyl and AU-Functionalized Surfaces

For DNA combing to work on this micropatterned surface, the DNA ends need to be attached preferentially to the octenyl-functionalized surface. Dynamic molecular combing (coverslip withdrawn from a reservoir) is the most widely used method of generating such receding meniscus among others including gravity, dragging, capillary flow, gas pressure, wicking with filter paper, and evaporation. The DNA adsorption and linearization on octenyl-functionalized and AU-functionalized surfaces were first compared. Parallel, linear individual molecules adsorbed to octenyl surface in an orientation perpendicular to the receding meniscus, while on AU surface, DNA molecules were found to be adsorbed in a globular form. This is consistent with the fact that a coiled DNA molecule was expected to adsorb at multiple points along its backbone through the electrostatic attraction between the negatively-charged DNA backbone and the weakly cationic AU layer. In order to linearize the DNA molecules on an AU-functionalized surface, a concurrent shearing flow was necessary to generate linearization at an adequate rate compatible with the adsorption kinetics between DNA and alkylamines. Clearly, preferential attachment of DNA ends to octenyl-functionalized surface is critical for the dynamic combing.

### Fabrication Parameters Affecting DNA Attachment

A micropatterned octenyl/PEG surface was designed in part to alleviate the complications of DNA combing such as DNA aggregations and high fluorescent background of salinized substrate. FIG. 1B shows a schematic of such a micropatterned surface. A “binding region” on the substrate is silanized with the octenyl functional group to promote DNA end-attachment. The “extending region” is functionalized with PEG for DNA linearization and observation. This region was incorporated to minimize non-specific free stain and dNTP adsorption. The spatial ratio between these two regions can be controlled to select for a targeted molecular size and to control combing density for fewer intermolecular crossing events and reduced intramolecular loop formation for the best observation and interrogation conditions.

To characterize the silanization process, the contact angles on octenyl and PEG regions were measured using Surface Analyst 3001 (BTG Labs). After glass substrates were grafted with OTMS, activation of the surface using air-plasma (200 W, 3 min) yielded a contact angle of 73° (reaction time, 4 h). Piranha-activation followed by overnight silanization resulted in substrates with marginally higher hydrophobicity (contact angle, 76°) but this was accompanied with reduced reproducibility. Contact angle measured on a 10-10 micropatterned substrate after PEG-grafting (PTMS, 32.5 nM) was found to be 26° in the PEG-only region and 45° in the patterned region. Different contact angles confirm the presence of contrasting surface functional groups, octenyl and PEG. Interestingly, contact angle on the 10-10 substrate, that has an even distribution of the two modifications, was in between the contact angles on octenyl and PEG-coated surfaces.

Photolithography soft-bake temperature as well as PEG-silane concentration were found to affect DNA attachment. To assess the impact, photolithography was performed on two octenyl-coated glass substrates with different soft bake (without post-exposure bake) temperatures, 95 and 115° C. PR was stripped, and substrates were cleaned thoroughly before combing T7 DNA followed by visualization. For the substrate baked at 115° C., DNA density was observed to be lower in the previously PR-covered region than in the PR-stripped region. However, the 95° C.-baked substrate had similar DNA densities on both, previously resist-covered and resist-stripped regions. This interaction between unexposed PR (SC1813) and octenyl functional group (or any silane) at 115° C. has not been reported previously.

To ascertain that the PR thin film shielded underlying octenyl layer from plasma treatment, T7 DNA was combed on micropatterned substrates that were plasma-treated, PR-stripped and cleaned. DNA combing density on the octenyl region remained unaffected compared to that observed on substrates that were not treated with plasma. Moreover, there was no DNA attached to the activated glass surface indicating a high degree of hydrophilicity.

The optimum PEG-silane concentration was found to be was 32.5 nM. At higher concentrations (>240 nM), DNA combing density was found to decrease dramatically, likely due to parallel reaction with unreacted methoxy groups (or hydroxyls) in the octenyl region. Higher DNA concentration (3×) in the combing reservoir did not improve DNA density significantly.

### DNA Linearization on Micropatterned Substrate

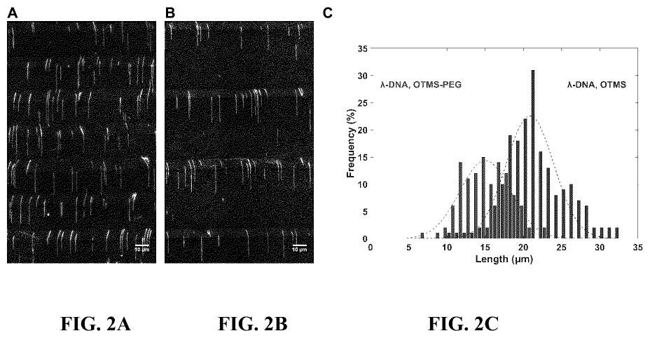

A micropatterned glass substrate with 10 μm wide octenyl and 15 μm wide PEG sections (10-15) was combed with λ bacteriophage DNA. The substrate was immersed into λ-DNA solution for an extended incubation time (compared to an unpatterned octenyl substrate) of 15 minutes, after which it was withdrawn at 0.1 mm/s, dip-stained with a reservoir containing YOYO-1, and imaged (FIG. 2A). The octenyl sections appeared brighter than adjacent PEG sections, due to adsorption of YOYO-1. On an average, more than 98% of the combed DNA molecules extended with one end bound to the upper octenyl section. It is interesting that we observed very few molecules with two ends attached to the same octenyl section forming a loop. Similar results were obtained with λ-DNA linearized on a 10-40 substrate albeit with lower combing density due to reduced effective area of binding (FIG. 2B). Linearized molecules extending from PEG to octenyl section appeared bent, but no such bends were observed on molecules extending from octenyl to PEG section. We surmised this was due to the concave meniscus in polypropylene DNA reservoir.

To evaluate the stretching factor (s.f.) of DNA on OTMS-PEG substrate, λ-DNA was combed on a 10-40 substrate as well as on an unpatterned OTMS substrate. DNA backbone length measurements on the unpatterned substrate yielded a peak at 21 μm (FIG. 2C, blue), corresponding to an s.f. of 127%. For the 10-40 substrate, backbone lengths were measured separately for each silanized section, l_{OTMS} and l_{PEG} respectively. The histogram plotted for the combined length, l_{OTMS}+l_{PEG} (lₒᵥₑᵣₐₗₗ), and fitted with a gaussian curve, yielded a peak at 14.7 μm (FIG. 2C,), which corresponds to an s.f. of 89%. Further, on 10-40 substrate, we assumed 127% stretching in the octenyl section, and derived the s.f. on PEG section using the equation below.

$$s . f {.}_{P E G} = \frac{{l}_{P E G}}{( 16.49 - ( \frac{{l}_{O T M S}}{1.27} ) )}$$

The resulting mean s.f. on PEG section was found to be ˜84%. This clearly reflects the overall reduction in s.f. due to PEG surface modification. By increasing density of the grafted PEG, we may potentially be able to under-stretch the DNA further. In general, the micropatterned substrates produced marginally higher stretching uniformity compared to unpatterned OTMS substrates, with standard deviations of 3 μm and 4.1 μm, respectively. Additionally, individual molecules were observed to be less aggregated on OTMS-PEG substrates compared to OTMS substrates.

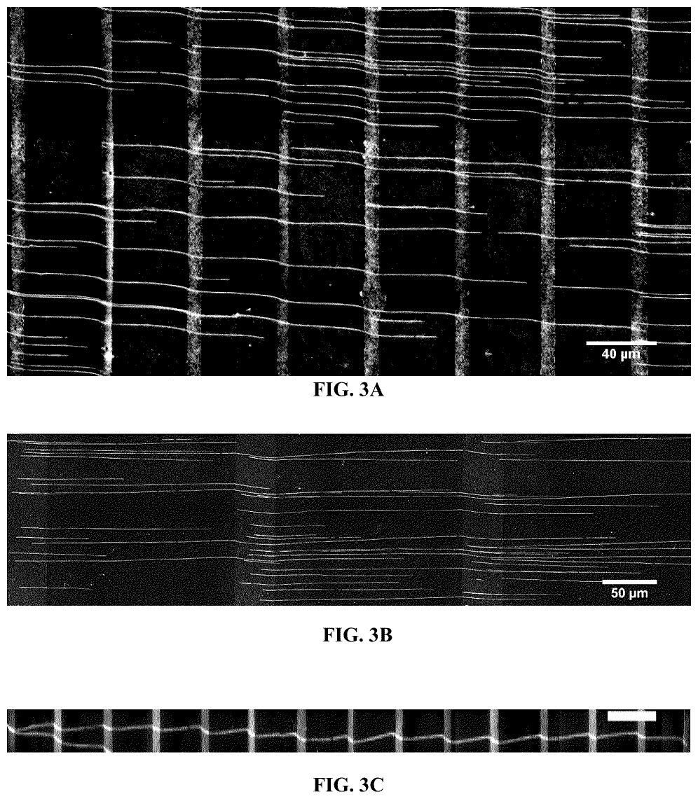

Further investigated was the linearization of long human DNA (hgDNA) molecules onto OTMS-PEG substrates. Typical resulting images are shown in FIGS. 3A-3B. As shown in FIG. 3A, the tail ends of long hgDNA preferentially bound to octenyl sections of a 10-40 substrate. Out of 326 molecules measured, fewer than 24 had their leading end bound to PEG section instead of an octenyl section. The combed hgDNA molecules were also more orderly with very few molecules crossing each other. Fewer loops were observed compared to combing on an unpatterned substrate, possibly due to the reduced chance of two-end binding events occurring in a given 10 μm octenyl section. FIG. 3B shows similar combing results on a 40-170 substrate, with lower binding density. Table 1 summarizes the average lengths of combed DNA on 10-40 and 40-170 substrates, calculated with a lower threshold set at 100 kbp. Here, the s.f. value obtained from λ-DNA measurements on 10-40 substrate was used to calculate the average lengths in kbp. On the 10-40 substrate, 84.42% of the molecules were longer than 300 kbp with average at 677 kbp, and over 20% of them were above 1 Mbp in length. DNA molecules combed on the 40-170 substrate were generally longer, with 32.4% over 1 Mbp. Very long (>1 Mbp) molecules using these longer pitch micropatterned substrates were routinely observed. One DNA molecule approximately 2 Mbp long is shown in FIG. 3C.

TABLE 1
Molecular size distribution of human DNA combed on
10-40 and 40-170 micropatterned glass

| | 40 μm pitch | | 170 μm pitch | |
|---|---|---|---|---|
| | | Mean length | | Mean length |
| Length (kbp) | % | (kbp) | % | (kbp) |
| 300 | 84.42 | 677.96 | 86.91 | 783.2 |
| 500 | 60.75 | 887.84 | 67.06 | 1045.38 |
| 1000 | 20.00 | 1460.91 | 32.40 | 1601.9 |

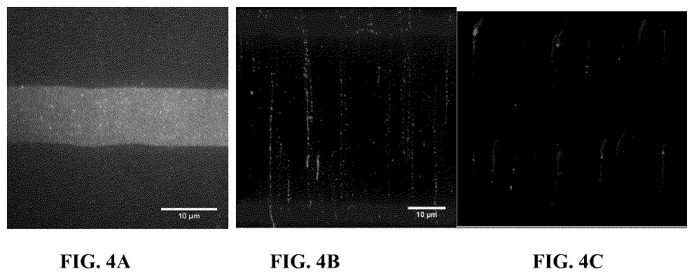

Table 1: Molecular size distribution of human DNA combed on 10-40 and 40-170 micropatterned OTMS-PEG substrate. Nested length distributions obtained from the dataset used for FIGS. 4A-4C are shown. The percentage of molecules measured above a threshold length (left column) is shown in the left column of each distribution. The mean length of the molecules above each threshold is shown in the right column. Both patterns produced long molecules, averaging 610.17 and 704.88 kbp at the lowest threshold for the 10-40 and 40-170 patterns, respectively. 2.49% more of the molecules combed on the 40-170 were above the 300 kbp threshold compared to the 10-40. This difference progressively increased with the threshold value to 6.31% at 500 kbp and 12.40% at 1 Mbp.

### Efficient Enzymatic Reactions on PEG-Passivated Surface

The OTMS-PEG substrates when viewed on epifluorescence microscope at high intensity illumination (473 nm, 100-150 mW; 532 nm, 150-500 mW) barely presented any autofluorescence to enable distinction between the PEG and octenyl sections. As noted above, YOYO-1 dye molecules adsorb more to octenyl sections relative to PEG sections. To further verify reduction in adsorption of fluorescent dyes in the PEG sections, a micropatterned 10-20 substrate was incubated with a solution containing ATTO-532-dUTP (100 nM). After washing out the free dye-nucleotides from the surface, the fluorescence intensity in the octenyl section was found to be about fifteen times higher in the PEG section. One can easily observe more distinctive bright spots in octenyl sections (FIG. 4A). This may have been due to hydrophobic-hydrophobic interactions between fluorescent moieties and the octenyl group compared to their non-interaction with the electrically-neutral and hydrophilic PEG functionality.

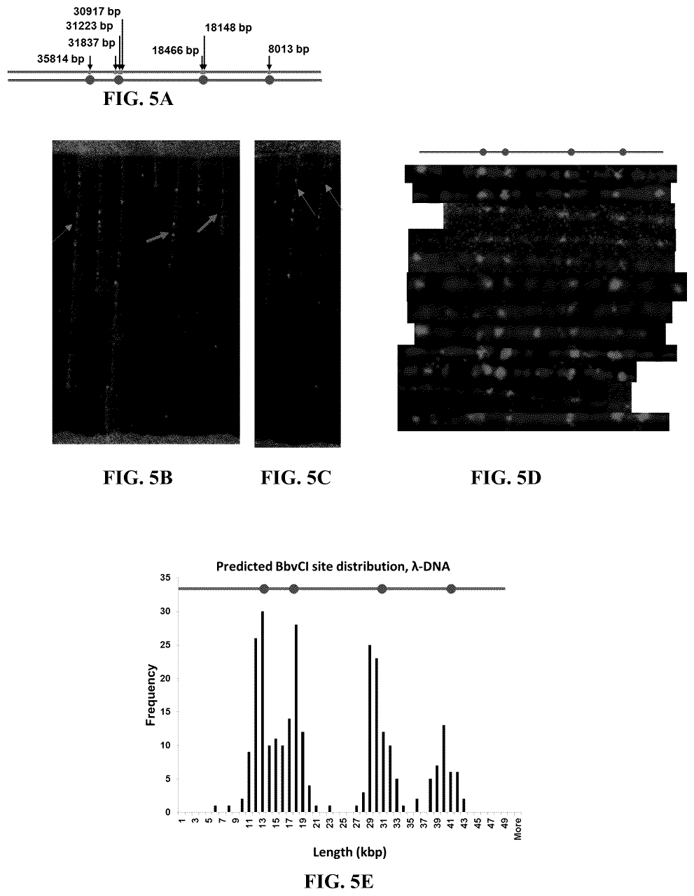

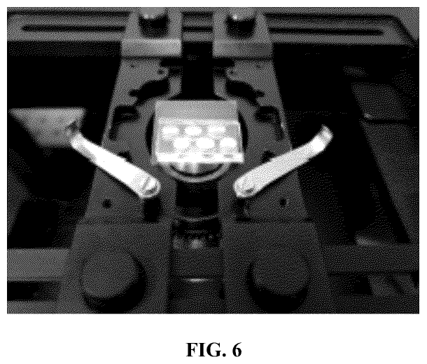

RNA transcription of T7 DNA on micropatterned surface was then tested. An evaporating oil, 1-dodecanol, was used to obtain non-overstretched DNA molecules (close to 100% of T7 DNA contour length). It was observed that dodecanol residue after combing, did not evaporate over time at room temperature or when oven-dried (65° C.) for 4 min. Moreover, reusing the same DNA reservoir with a floating dodecanol layer was not practical. By manipulating the common interface between DNA solution, combing substrate and air (triple-phase contact line) via surface modification, a high density combing of non-overstretched T7 DNA was achieved. After DNA combing, the transcription reaction on a 10-15 OTMS-PEG substrate could be performed. The results showed T7 RNAP successfully interacted with DNA molecules and was able to locate promoter sites to initiate transcription (FIG. 5C). Some of the DNA molecules (blue) exhibited anywhere from 1 to 4 bright spots (red). To confirm T7 RNAP was indeed the reason for labeling, control experiments were done in parallel following the exact same procedures and using all the same reagents besides T7 RNAP enzyme. No labeling was present in any of the control experiments.

To test if two successive enzymatic reactions may be performed on the micropatterned substrate, nick-labeling was performed on hgDNA molecules linearized on a 10-40 substrate (FIG. 4B). Nick-labeling consisted of two consecutive reactions-nicking using Nt.BspQI for 1h at 37° C. followed by labeling with DNA Pol I for 1 h at 37° C. After each reaction, the surface of microliter-well was washed gently to remove the enzyme and dye-nucleotide molecules. The substrate was then imaged for ATTO-532 followed by YOYO-1, and the two images superimposed to form a composite image. Most of the aggregated red spots (ATTO-532-dUTP) were observed along the blue DNA backbone in the PEG section (FIG. 4B). Much fewer free dye molecules randomly adsorbed outside the DNA backbones, indicating dye-nucleotide incorporation. Multiple long labeled DNA molecules that spanned across the 40 μm (˜138.4 kbp) PEG section were observed in every image. In a control experiment with the same conditions but without nickase (Nt.BspQI), minimal fluorescent labeling along the DNA backbone was observed. This shows that both enzymes were active on the PEG surface and can be used successively. Although the reactions were found to be efficient across several trials in separate microliter-wells, the amount of combed DNA on the surface depleted significantly in most of the wells, particularly post nicking reaction. Thereafter, a layer of polyacrylamide (PA) gel was casted atop the combed DNA, before proceeding with any chemical reaction. The added PA layer not only helped fix the DNA by minimizing harsh physical flow forces during handling but also provided a consistently aqueous environment to conduct reactions.

Taken together from the above experiments, the PEG sections not only significantly reduce the random adsorption of free fluorescent dyes but is also amenable to enzymatic reaction.

### On-Surface Nick-Labeling and Mapping of λ-DNA

To demonstrate on-surface DNA mapping via fluorescent nucleotide incorporation, λ-DNA was used as a model genome and nick-labeled at the seven BbvCI sites (FIG. 5A (i), backbone is blue, BbvCI sites are shown). Nicking was performed using Nb.BbvCI for 2 h at 37° C. followed by labeling with Klenow Fragment (3′→5′ exo-) at 37° C. for 2 h. After each reaction, the microliter-well was washed thoroughly with 1×CutSmart Buffer and 1×NEBuffer 2.0, respectively. Imaging was performed on a fully-automated epi-fluorescence microscope, before and after staining with YOYO-1. Each addressed location on the micropatterned substrate was autofocused and imaged for ATTO-532 and for YOYO-1 successively, and the two images superimposed to produce a false-color composite image (FIGS. 5B-5D). The octenyl sections appeared very bright due to the strong adsorption of YOYO-1 dye. FIGS. 5B and 5C are raw images. The single λ-DNA molecules are combed starting from a random location in the 10 μm octenyl section. As can be observed in FIGS. 2A and 2B, a substantial number of them combed beginning from the top of the octenyl section limiting the length of backbone available in PEG section for labeling. The λ-DNA was concatemerize by briefly heating to 65° C. for 10 min followed by 1 h incubation at 37° C. to increase the chance of observing fully labeled λ-DNA. The arrows in FIGS. 5B and 5C point to individual λ-DNA molecules with full BbvcI pattern, while the arrow indicates molecules with partial pattern. Nearly all the labels observed colocalized with the DNA backbone confirming that the aggregated fluorophores are indeed incorporated ATTO-532 nucleotides.

Hence, to begin analysis, labeled λ-DNA molecules were identified by delineating a rectangle 60 px in height (corresponding to end-to-end distance between farthest BbvCI sites, 27.8 kbp) with an arbitrary width, to act as a reading frame, in randomly selecting molecules with at least 4 labels within the boundaries of the rectangle. These molecules are shown in FIG. 5C and used to generate the histogram in FIG. 5D. It can be observed that there are a few false positives, and most of the molecules do not have both the end-labels.

Each peak in FIG. 5E corresponds to experimentally measured distance between adjacent BbvCI sites, normalized by the total distance between the farthest BbvCI sites. A total of 150 molecules were selected with the above criteria. Molecules with at least one end-label in addition to the four BbvCI labels totaled 39 and were used to calculate the predicted positions of BbvCI sites. Overall, the peaks (predicted site positions) match closely the BbvCI site locations on λ-DNA.

This is the first report of on-surface fluorescent labeling and mapping of long DNA molecules that has the potential for adaption to high-throughput whole genome mapping, with the flexibility to perform multiple cyclic enzymatic reactions on fixed DNA. Going further, whole genome as well as targeted single DNA interrogation should be possible on this platform, such as multi-color mapping and base-by-base sequencing. As noted in previous section, stretching of λ-DNA was less uniform than in nanochannel arrays, but the flexibility to perform multiple labeling steps on fixed DNA is highly significant and can open up new ways to analyze DNA sequence.

### EXAMPLE 2

### Method

Clonal human DNA template, RP11-1116M14, was used to perform proof-in-principle experiments. For this, DNA was combed on a micropatterned (˜8 μm-wide ‘DNA binding’, ˜42 μm-wide ‘DNA-passivating’) glass coverslip. Circular holes were punched through a PDMS slab which was bonded to the coverslip. The device now contained 5-6 microliter reactor wells that can be operated independently. The chip is then mounted atop the microscope stage for image capture. In some instances, reaction and wash buffers have been introduced using a syringe pump setup and a modified PDMS layer (flow cell), while the chip was held on the microscope throughout the experiment. EnGen® Spy dCas9 (SNAP-tag®) was purchased from New England Biolabs. Fluorophore-tagged tracrRNA (Atto-550 and Alexa Fluor 647N) was purchased from Integrated DNA Technologies. Multiple probes were designed in-house, to target RP11-1116M14 as well as human genomic data. After validating the design from a reference map incorporating tolerance factors known to affect dCas9 targeting, crRNAs were ordered from GE and Integrated DNA Technologies.

After complexing the crRNA (probe containing the target sequence) with tracrRNA (universal sequence tagged with fluorophore) to result in guide-RNA complex (gRNA), dCas9 is added to complex with the gRNA. Further, this solution was added to the designated well containing combed DNA molecules to perform labeling. Imaging was either performed with or without the evacuation of the well, as well as before or after DNA backbone staining with YOYO-1.

In the experiment to demonstrate two cycles of labeling on a single DNA molecule observed in real-time, protease purchased from Qiagen was used to break down the dCas9-gRNA complex from the first labeling step. After this, protease solution was evacuated and washed multiple times before introducing the second dCas9-gRNA complex.

Reference maps were generated using Basic Local Alignment Search Tool (BLAST) and SAMtools for the analysis of experimental data.

### Results

The length of the template DNA (bacterial artificial chromosome, BAC), RP11-1116M14 (M14 in short), is around 160 kbp including regions of bacterial genome that it was cloned with. This translates to 54.4 μm when stretched to true length (100% stretched). In the combing experiments with the device, a stretching factor of nearly 1 is achieved, validated using λ-phage DNA (48.5 kbp). The width of the DNA-passivating region was chosen (42 μm) to allow for maximum length of DNA template to be probed by the labeling chemistry.

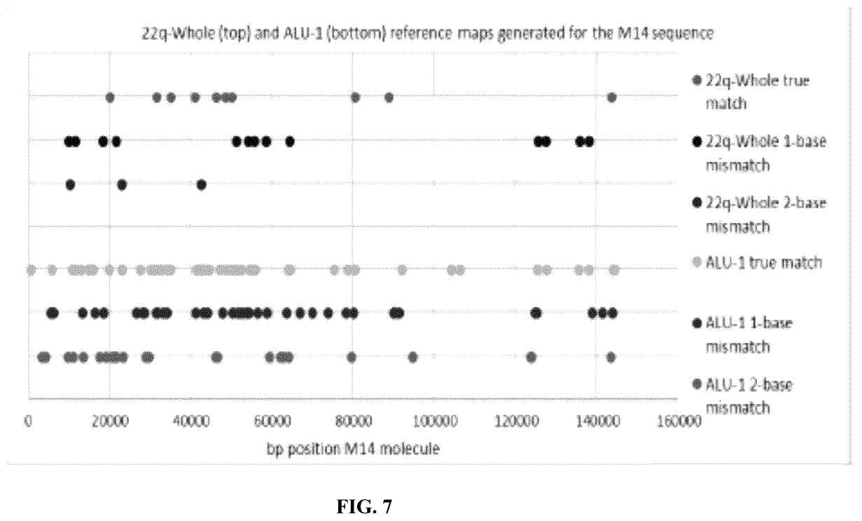

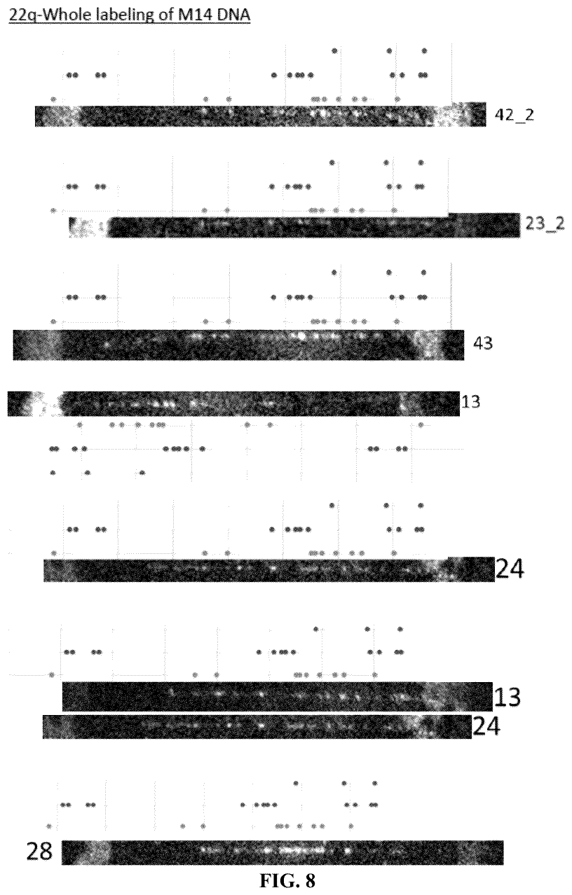

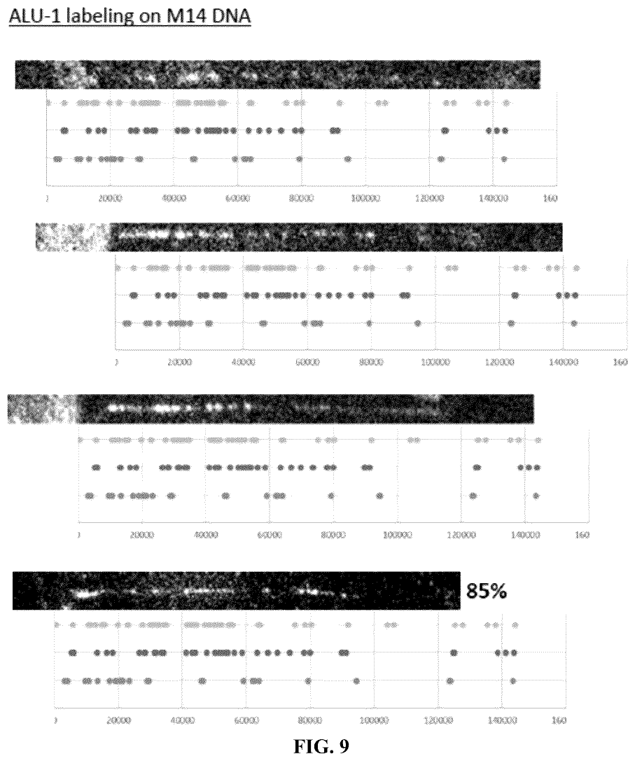

To map single M14 molecules, two repeating motifs were targeted, one of which (ALU-1) is relatively more frequent and results in denser clusters of target sequences than the other (22q-Whole). The ALU-1 probe has been designed to target the Alu element, the most abundant repetitive element comprising around 11% of the human genome. The reference map generated with the ALU-1 probe is shown in FIG. 7 (bottom), along with 1-base mismatch and 2-base mismatch positions. The 22q-Whole probe was designed to target repetitive elements across the entire genome, with a particularly high abundance on the q arm of chromosome 22. The 22q-Whole probe has multiple sites with single motif per kbp (approx. resolution limit of microscope), which serves to simultaneously validate fluorophore detection sensitivity.

Around 50 mm² area of the glass surface area was scanned (4-5 wells) to obtain images for each labeled-DNA species. Images were captured in the TIRF configuration, by manually scanning for single DNA molecules stretched fully across the DNA-passivating region.

The obtained images were analyzed using ImageJ by aligning single molecules against the respective reference map (FIG. 7). Owing to different residence times of the dCas9 complex on the DNA backbone, some labels were observed at sites with a single base mismatch and even two-base mismatch sequences. This specificity tolerance of dCas9 is in line with reported literature.

The images of molecules aligned against the reference maps are shown in the panels below. Each molecule is indexed and compared against the reference assuming 100% stretching, i.e. no over-stretching or under-stretching, although not all molecules will be 100% stretched.

### Proposed Sequencing Method Using Reversible Terminator Chemistry

Long (>1 Mbp) backbone-stained single DNA molecules are linearized using the proposed microchannel device in an aqueous buffer. The excess backbone-staining dye is washed away with fresh buffer, and the location of DNA molecules on device surface is registered using an automated XYZ stage and microprocessor.

An equimolar mixture of individual (A, T, G, C) fluorophore-tagged reversible terminator nucleotides is prepared in an aqueous buffer with added DNA polymerase enzyme. In the first cycle, the master mix is introduced into the microchannel to initiate single base incorporation at sequence-specific nicked sites (enzymatic) or randomly generated single strand breaks (enzymatic or heat). After the incorporation step, the excess master mix is cleared out and the channel is washed using a wash buffer. At the registered positions of DNA molecules, fluorescence signal is collected on all four imaging channels, with a base call made based on the fluorophore detected. Subsequently, a second cycle of single base incorporation is carried out, washed, and imaged. This process will continue until desired or until read errors begin to increase. Typically, using this chemistry on bound-DNA templates, read length is 300 bp and above.

The above method is used to sequence DNA at regions along a single long molecule. The additional co-locational information of sequenced regions enables accurate (high confidence) mapping/assembly of the sequenced fragments. This method of measurement is not only unique but also provides valuable genetic data in disease diagnosis.

In one instance, DNA sequencing is initiated at specific sites across the long molecules simultaneously using nickase enzymes. After single nucleotide incorporation and detection, this step is repeated multiple times to sequence the hotspots on individual DNA molecules.

In another instance, the DNA sequencing is initiated at several random sites across the long DNA molecules simultaneously, either by nucleases, heat or UV exposure. After single nucleotide incorporation and detection, this step is repeated multiple times to sequence DNA. At the end, the DNA backbone is stained with an intercalating dye, and visualized under a multichannel fluorescence microscope. This will define the linkage between the sequencing reads.

### EXAMPLE 3

### Using Crispr-Cas9 Labeling to Interrogate Individual Base, and Tag Specific Genomic Region of Interest

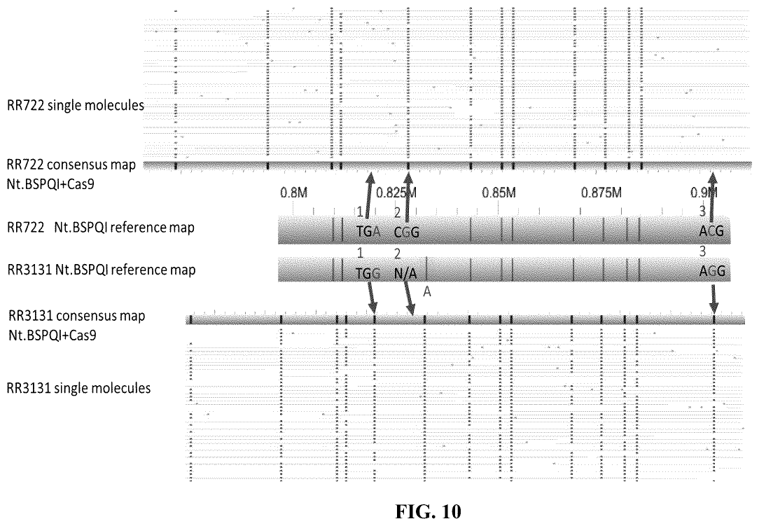

The main strategy for long-range optical mapping is based on measuring the distances between the short sequence motifs recognized by nicking endonucleases (6-8 bp) on single long DNA molecules. The key information is the pattern of distances between motifs. Current labeling strategies can only detect single-base differences at polymorphisms that happen to coincide with nickase motifs, which has limited the potential applications of optical mapping. For example, the H. influenzae strains RR722 and RR3131 share a 100 kb region (819-916 kb of RR722, NC_000907, and 884-981 kb of RR3131, NC_007416) with 99% sequence similarity. The Nt.BspQI sequence motif maps for the two strains are almost identical for this region, except for one extra nick of the RR3131 genome, due to an adenine single-nucleotide difference from RR722, thus the nicking enzyme labels the RR3131's allele but not RR722's allele (FIG. 10).

A strategy was devised to use multiplexed CRISPR-Cas9 labeling to distinguish single-nucleotide variants affecting 3′-NGG PAM sites since the editing system has a strong requirement for the PAM immediately following the 20 bp recognition sequences. Genetic variation impacting PAM sites (i.e. if one of the G bases of a PAM in one genome is variant in another) is expected to strongly impact labeling, even if they share the 20 bp recognition sequence. Thus, it is predicted that strong differential labeling at gRNA-guided PAM variants could reliably differentiate the single base difference between two genomes over long distances.

To demonstrate single-base resolution of multiplexed CRISPR-Cas9 labels at variation affecting PAM sites, gRNAs targeting three distinct 20mer recognition sequences were designed, but for each one of the two H. influenzae strains lacked a 3′-NGG PAM signal due to single nucleotide variation (Table 2). Labeling by both Nt.BspQI and CRISPR-Cas9 were performed in a single tube reaction, and the results of optical mapping are shown in FIG. 10.

Single-base variation away from either G in the PAM nearly eliminated the corresponding labeling. At “locus 1” (NTHI0914-hypothetical protein of RR3131 and HI_0755-conserved hypothetical protein of RR722), the two strains share the same 20 bp recognition sequence (5′-AAAAATTGCTGCATCTTCTT-3′(SEQ ID NO: 427) as the gRNA, but RR3131 has a 3′-TGG PAM sequence, while RR722 has a TGA sequence instead. CRISPR-Cas9-mediated optical mapping clearly shows high-efficiency labeling at position 885289 in RR3131 (˜90% labeling), whereas RR722 molecules totally lacked labels (0%) at position 819899 (red arrow at “locus 1” in FIG. 10). Similarly, at “locus 3” (NTHI0947-50S ribosomal protein L29 of RR3131), the labeling difference between two strains can only be explained by the presence of alternative alleles in the two strains, in which RR3131 becomes labeled at 98698 with a perfect AGG PAM sequence; RR722 is not labeled at the syntenic position because of an ACG variant non-PAM sequence. At “locus 2” (ribB), the sgRNA matches RR722 at 828196 with a CGG PAM sequence, and correspondingly, over 90% of molecules spanning the position were labeled (red arrow at “locus 2” in FIG. 10). In RR3131, no labeling was seen at the best-matching genomic position (893590), but in addition to a non-PAM 3′-end (CTG), the first and third positions were also mismatched.

In summary, labeling efficiency was over 90% for gRNAs with an NGG PAM sequence, whereas almost none of the molecules were labeled if there is an alternative allele in the PAM sequences. This is in contrast to the variable labeling efficiencies seen for different mismatches from the 20 nt recognition sequences in the sgRNA experiments below. These results suggest that a customized optical mapping using gRNAs to target many of these polymorphisms (or “PAM SNPs”) could be an effective means to define long-distance haplotype structure in human genomes. It could also be applicable in other sample types, particularly mixed microbial specimens. The new DLE labeling strategy (6 bp motif) from BioNano genomics provides 50% more labeling site than Nt.BspqI labeling (7 bp motif) in human genome and potentially other genomes, which may resolve some haplotype features. However, the density of 1 snp per megbase is not enough to construct the whole-genome haplotype based on SNPs considering the the average DNA length of 300 kb.

An in silico analysis of whole genomes from the 1000 genomes project (36,37) was performed to determine the potential number and distribution of heterozygous PAM SNPs in the human genome, Out of 161 million NGG sites in hg38, on average, there are 220,000 heterozygous PAM SNPs in a single diploid human genome. In addition, there are on average 40,000 heterozygous indels (>4 bp) within potential CRISPR-Cas9 recognition sequences (20 bp+NGG); >2 bp heterozygous indels within the 20 bp gRNA recognition sequence preferentially target the matching allele. Together, the genomic density of these sites is ideal to generate long-distance haplotypes using CRISPR-Cas9 labeling of PAM sites with single molecules in these experiments longer than 100 kb.

TABLE 2
sgRNA Target sequences used for single base differentiation in FIG 10.
The differing bases are underlined for 3 locations.

| Strains | Locations | Loci | Target Sequence | gRNA Sequence |
|---|---|---|---|---|
| RR722 | 819899 | 1 | (SEQ ID NO: 421) | (SEQ ID NO: 427) |
| | | | AAAAATTGCTGCATCTTCTTTGA | AAAAATTGCTGCATCTTCTT |
| RR3131 | 885289 | 1 | (SEQ ID NO: 422) | |
| | | | AAAAATTGCTGCATCTTCTTTGG | |
| RR722 | 828196 | 2 | (SEQ ID NO:423) | (SEQ ID NO: 428) |
| | | | AACCATTCAAACGGCGATTGCGG | AACCATTCAAACGGCGATTG |
| RR3131 | 893590 | 2 | (SEQ ID NO:424) | |
| | | | CACTATTCAAACGGCTATTGCTG | |
| RR722 | 903309 | 3 | (SEQ ID NO: 425) | (SEQ ID NO: 429) |
| | | | AATATCCTTGCCTTGAGAGAACG | AATATCCTTGCCTTGAGAGA |
| RR3131 | 968698 | 3 | (SEQ ID NO:426) | |
| | | | AATATCCTTGCCTTGAGAGAAGG | |

### EXAMPLE 4

### Multiplexed sgRNA Preparation in a Single Tube Reaction

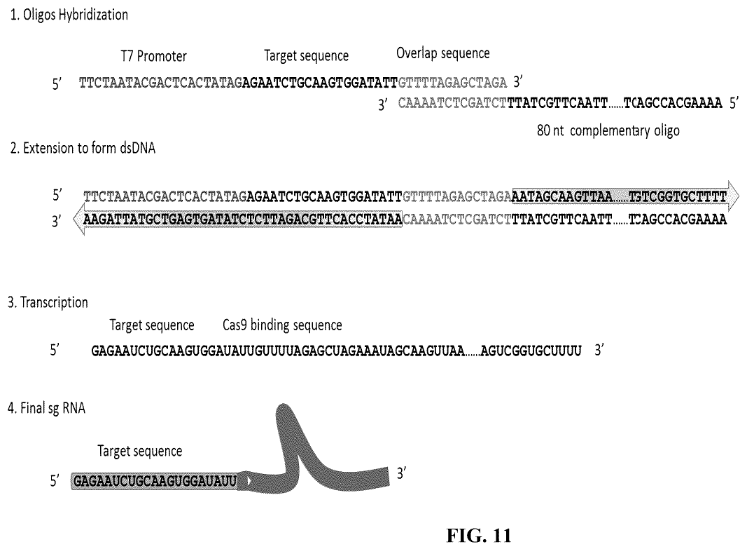

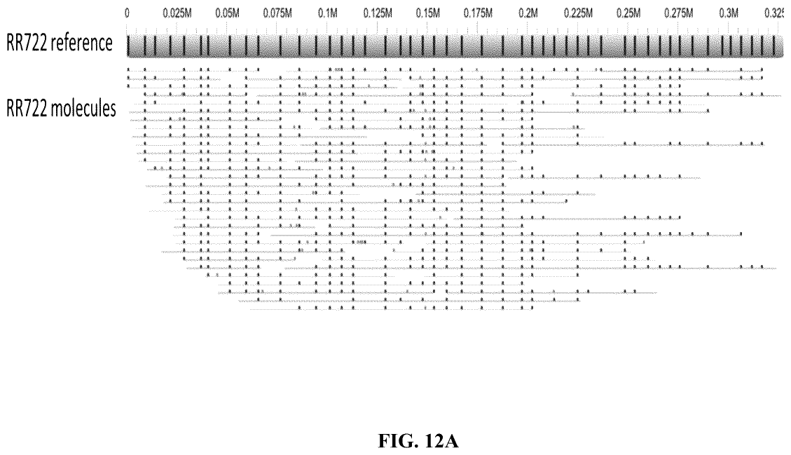

The previously described method to synthesize multiple sgRNAs in a single tube reaction was adapted. FIG. 11 shows the synthesis scheme and workflow. The key difference between the approach and the available commercial kit (EnGen® sgRNA Synthesis Kit, S. pyogenes from NEB) is a separate step to generate the dsDNA before the RNA transcription reaction. The mixture of multiple sgRNA oligos and the sgRNA complementary oligo was first mixed at a 1:1 ratio in reaction buffer. After Klenow exo-extension to generate dsDNA, the reaction was treated with Exonuclease I to remove extra ssDNA. The purity and size of dsDNA were further confirmed with gel electrophoresis before purification with PCR cleanup column. Typically 5 μg dsDNA at 0.2 μg/μl concentration is obtained. After sgRNA synthesis using T7 RNA polymerase, the sample was treated with DNasel to remove dsDNA and purified with an RNA cleanup column. Normally 40 ug sgRNA at 2 μg/μl concentration is obtained. This is enough to run ˜230 CRISPR-Cas9 labeling reactions with 300 ng target DNA sample each time. The purity and correct size of the dsDNA are critical to the synthesis of multiple sgRNAs 162. The sgRNAs were successfully synthesized in a single tube reaction.

### EXAMPLE 5

### Multiplexed sgRNA Optical Mapping

In the second customized mapping strategy, the mapping patterns were customized across a genome by selecting sets of specific single-guide RNAs (sgRNAs) for features of interest. This is particularly useful in designing different patterns to differentiate similar genomes or conserved sequences between strains or haplotypes. In designing the patterns, it is critical to avoid evenly distributed sgRNAs, because only long molecules across the entire pattern can be uniquely aligned. To test this, first a two custom optical mapping patterns were designed using the different H. influenzae bacterial strains, lab strain Rd KW20 (RR722), and a marked derivative of clinical isolate 86-028NP (RR3131) as the model systems.

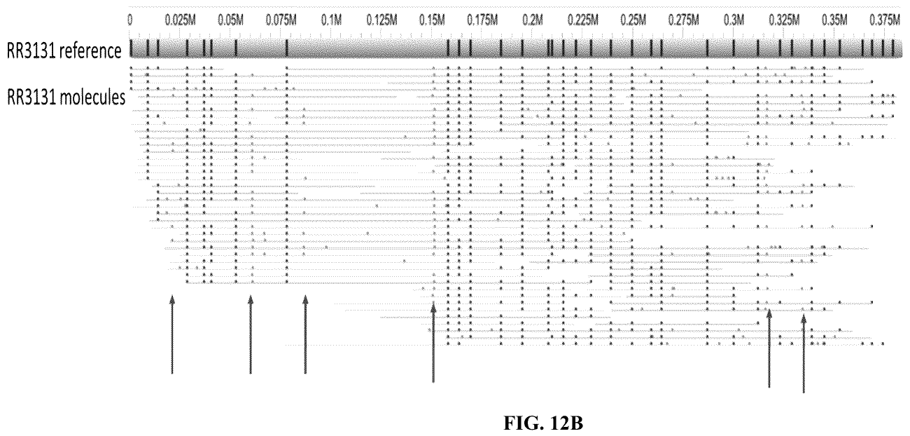

48 sgRNAs were designed to target a 300 kb region of RR722 (0-350 kb of NC_000907), which shares high sequence similarity with RR3131 strain (0-315 kb NC_007416). Each sgRNA was designed to have a single perfect match of 20 bases upstream of PAM NGGs based on the Rd reference genome (cr 1). These 48 sgRNAs are evenly distributed across the 300 kb region of RR722 (RR722 reference map in FIG. 12A). Dark lines on the bar indicate predicted sgRNA locations. Out of 48 sgRNAs, 33 sgRNAs also have a single perfect match of 20 bases upstream of a PAM NGG on the RR3131 strain. However, the predicted targeting locations of these 33 sgRNAs form an unevenly distributed mapping pattern (RR3131 reference map in FIG. 12B), indicative of structural variation between the genomes.

A single mixture of 48 sgRNA was then generated, which was used to label and map targeted regions in both the RR722 and RR3131 genomes. The individual molecules are indicated as thin lines that are aligned to blue references in FIGS. 12A-12B. The two data sets show similar characteristics with an average molecule length of 255 kb and 249 kb for RR722 and RR3131 respectively. But with the same amount of raw data, three times more molecules could be uniquely aligned to the RR3131 strain than the RR722 strain, even though RR3131 has fewer perfectly matched sgRNAs (FIGS. 12A-12B, respectively). This is due to the fact that the shorter molecules will generate ambiguous alignments to the evenly distributed patterns. Longer molecules are needed to map across the whole evenly distributed reference, which results in fewer molecules aligned to RR722 sgRNA map. This clearly shows that an unevenly distributed mapping pattern could result in better mapping.

### EXAMPLE 6

### Main Sources of Off-Target Labeling

CRIPSR-Cas9 tagging is prone to off-target labeling. It is important to reduce off-target labeling as much as possible, especially when trying to use custom-target mapping to map sequences with high similarity. The 48 sgRNAs (20 base recognition sequence) against the RR3131 reference were aligned. 15 sgRNAs out of the above 48 sgRNAs that have imperfect matches to the RR3131 genome. Some of them result in off-target labeling in RR3131. In FIG. 12B, many single molecules show off-target labels (light green dots) at six different locations, which are present in the RR722 genome, but not present in RR3131, therefore absent from the reference map.

7 of these 15 sgRNAs show several partial matches (<8 bases) across the 300 kb region, but without a PAM NGG next to the best match, which could not be labeled. These 7 sgRNAs are designated as “N/A” in Table. 4 and are not likely to contribute to off-target labeling. 6 of the remaining 8 sgRNAs were found to match the RR3131 reference around off-target loci with a PAM motif and a single mismatch in the 20 recognition sequences. These 6 are contributing to the off-target labeling and designated as “off-target” in Table 3. The final 2 sgRNAs of the 15 did not produce a label in RR3131 and are listed as “No label”. Of the two, the sgRNA at 219206 of RR722 ((SEQ ID NO: 442) TTGTTTTACGATATAATACGNGG) also shows a single base mismatch on RR3371 strain, but did not result in off-target labeling. The sgRNA at 323878 of RR722 (SEQ ID NO:444)(TAATCAAGCATTAGATAGCTNGG) has several mismatches close to the 5′ end and also did not result in off-target labeling.

All six sgRNAs that caused high-frequency off-target labeling had a single mismatch to the target sequences of RR3131. Five of six had the single mismatch close to the 5′ end, distal from the PAM sequences, except the sgRNA at 86065 of RR722 (SEQ ID NO: 434) (GTTACATTACACACAAACTINGG) with the single mismatch at the 3^{rd} base upstream of PAM. For example, the sgRNA at 21722 of RR722 ((SEQ ID NO: 430) (GCTTTTTAGGATATCGTCCCNGG)) is designed to target the RR722 genome at coordinate 21722, but it also matches a synthetic position in RR3131 (at coordinate 21698) with a single mismatch (G/A) at the 9^{th} base from the 5′ end. The off-target labeling of the RR3131 chromosome around 21698 was likely caused by this sgRNA. For the same reason, the sgRNA at 59529 of RR722 ((SEQ ID NO: 432)GCGGTATCCACCCCCACTGCNGG) likely generated the off-target labeling on RR3131 around 60913 with a single mismatch at the 3^{rd} base. Notably, the off-target labeling on RR3131 is more efficient with sgRNA designed for RR722 at 59529 locus than the sgRNA of RR722 at 21722 locus, which may reflect that its mismatch is closer to the 5′ end.

Overall, these results are consistent with the observation that the last 8-10 seed bases of sgRNA upstream of the PAM are more important for reducing the off-target labeling (38-41), and that multiple mismatches also reduce off-target labeling.

TABLE 3
The off-target labeling on RR3131. Two rows are shown for each of
8 probes that did not have a perfect hit in the RR3131 genome.
The second row is the designed probe named for its hit location
on the RR722 genome. The upper row is the sequence found in the
RR3131 strain, and named for its location. Bold indicates a PAM
sequence motif (NGG). Underline indicates a base that does not
match the designed probe. The last 2 probes did not have a label
seen consistently in the aligned data.

| Strains | Locations | Labeling | target Sequence |
|---|---|---|---|
| RR722 | 21722 | | (SEQ ID NO: 430) |
| | | | GCTTTTTAGGATATCGTCCCNGG |
| RR3131 | 21698 | off target | (SEQ ID NO: 431) |
| | | | GCTTTTTAAGATATCGTCCCAGG |
| RR722 | 59529 | | (SEQ ID NO: 432) |
| | | | GCGGTATCCACCCCCACTGCNGG |
| RR3131 | 60913 | off target | (SEQ ID NO: 433) |
| | | | GCAGTATCCACCCCCACTGCAGG |
| RR722 | 86065 | | (SEQ ID NO: 434) |
| | | | GTTACATTACACACAAACTTNGG |
| RR3131 | 86656 | off target | (SEQ ID NO: 435) |
| | | | GTTACATTACACACAAATTTTGG |
| RR722 | 94393 | | (SEQ ID NO: 436) |
| | | | GGGGCGTAAATTCTTAACATNGG |
| RR3131 | 151264 | off target | (SEQ ID NO: 437) |
| | | | GGAGCGTAAATTCTTAACATTGG |
| RR722 | 253327 | | (SEQ ID NO: 438) |
| | | | CGAAGGGATAAATATTGCGANGG |
| RR3131 | 316470 | off target | (SEQ ID NO: 439) |
| | | | TGAAGGGATAAATATTGCGATGG |
| RR722 | 270963 | | (SEQ ID NO: 440) |
| | | | TAGCACTTAAAAGAGGAATGNGG |
| RR3131 | 334078 | off target | (SEQ ID NO: 441) |
| | | | TGGCACTTAAAAGAGGAATGGGG |
| RR722 | 219206 | | (SEQ ID NO: 442) |
| | | | TTGTTTTACGATATAATACGNGG |
| RR3131 | 281336 | no label | (SEQ ID NO: 443) |
| | | | TTGTTTTGCGATATAATACGAGG |
| RR722 | 296956 | | (SEQ ID NO: 444) |
| | | | TAATCAAGCATTAGATAGCTNGG |
| RR3131 | 359914 | no label | (SEQ ID NO: 445) |
| | | | GCGTAAAGCATTAGATAGCTTGG |

### EXAMPLE 7

### Customized Optical Mapping of a Whole Bacterial Genome

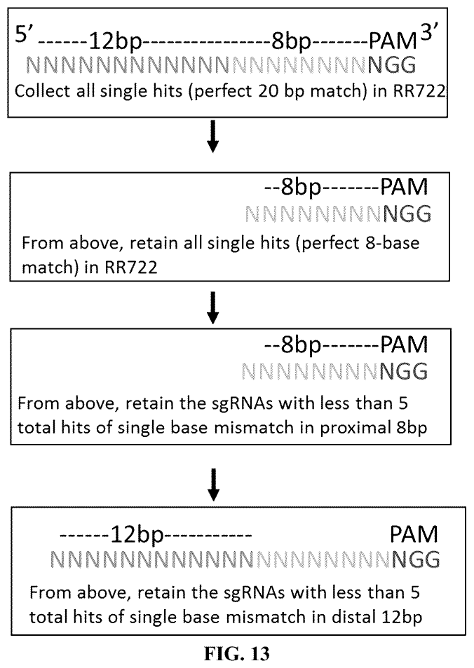

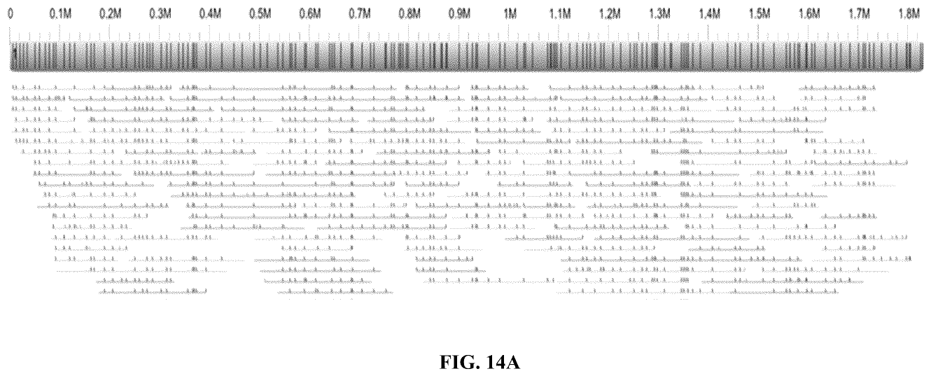

Based on the target labeling results and the reports that 8 seeding bases immediately upstream of the PAM sequence (NGG) have higher discrimination, the design pipeline was optimized to select a set of sgRNAs spanning the full RR722 genome in a series of four stepwise filters: a) collected all possible sgRNAs with a single perfect match to the RR722 reference (all 20mers followed by a 3′ PAM NGG that occur only once in RR722) were first collected; 40870 such possible sgRNAs were available. (b) From those, only the 8-base seeding sequences proximal to the PAM with single perfect hits to the reference were collected. If an 8-base seed had multiple perfect hits to the reference, it was discarded since these had a high chance of contributing to off-target labeling. The remaining sgRNAs (15339) all had a single perfect hit of 20 bases and a single perfect hit of the 8-base seeding sequences. (c) Since all 8 base-seeding sequences have multiple hits with a single mismatch, a third filter was then applied to minimize the number of hits in the 8-base seeding sequences with single mismatches to RR722. This resulted in 1,507 gRNAs with <5 singly mismatched hits in all 8-base seeding sequences. (d) From this dataset, off-target nicks were further minimized by keeping the sgRNAs with one more mismatch in the first 12 bases from the 5′ end (415 remains). The sgRNA design flow chart is summarized in FIG. 13. The final set of sgRNAs have only one perfect hit across the RR722 reference sequence in their 20-base recognition sequences and less than 5 hits with a mismatch in the 8-base PAM-proximal seeding sequence and another mismatch in 12 bases from the 5′ end respectively. After the four filters to minimize off-target labeling, a final manual adjustment was made to avoid evenly distributed mapping patterns. This resulted in a final set of 162 gRNAs (Table 5) with an average density of 9 predicted labels per 100 kb on RR722. The labeling density is similar to Nt.BspQI labeling density used in commercial optical mapping kits (1).

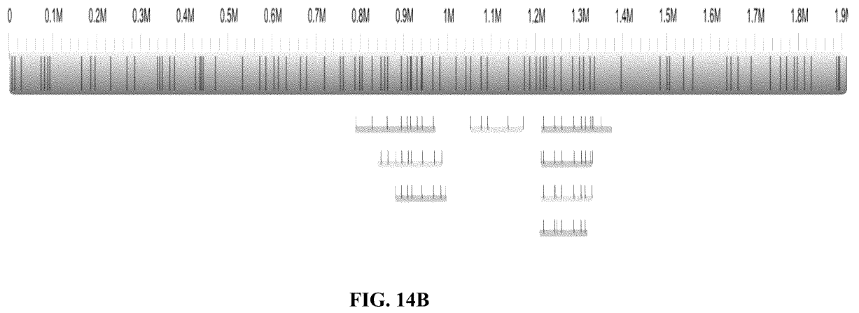

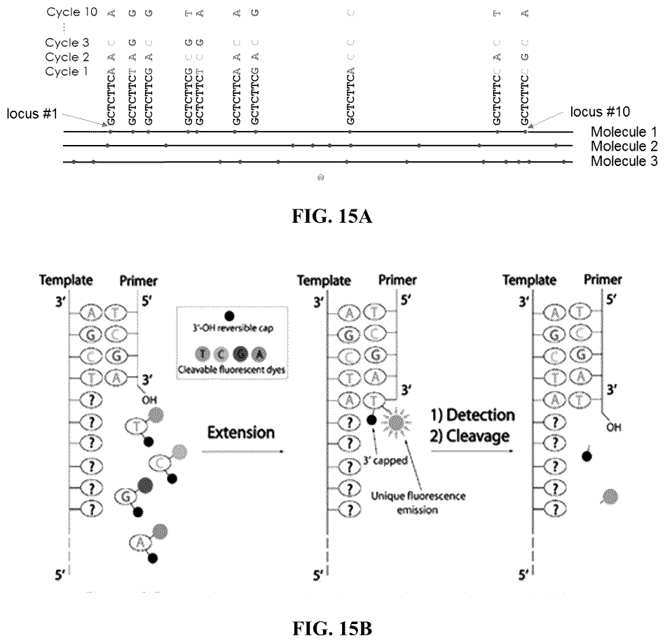

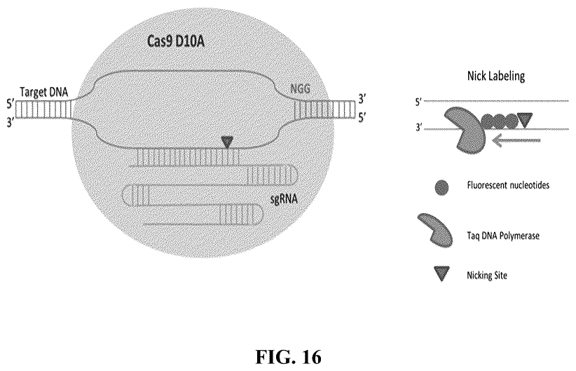

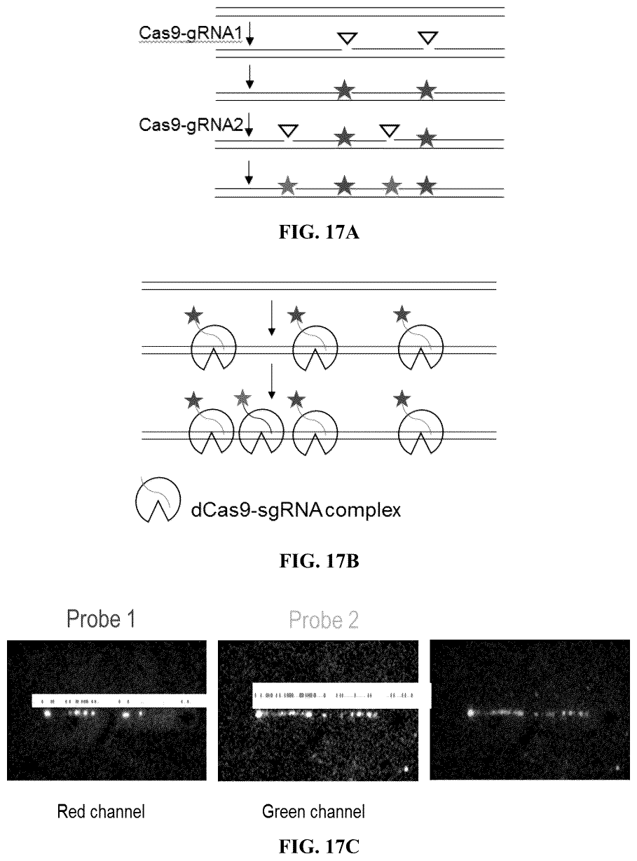

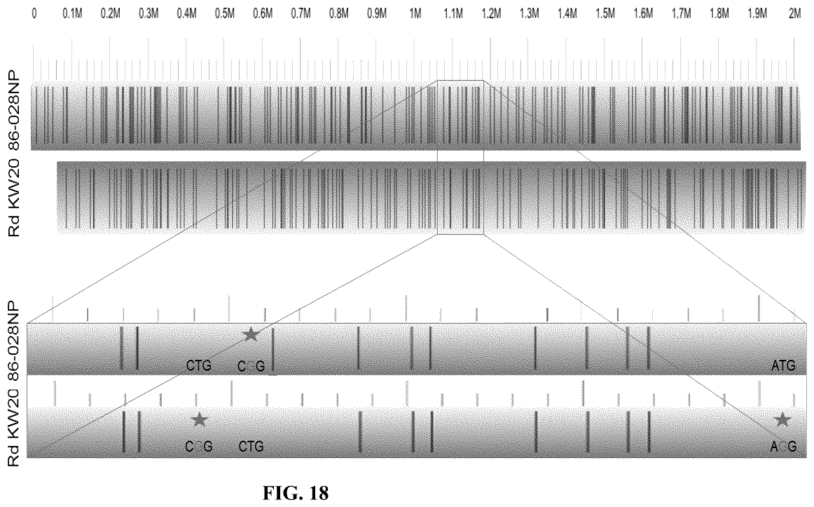

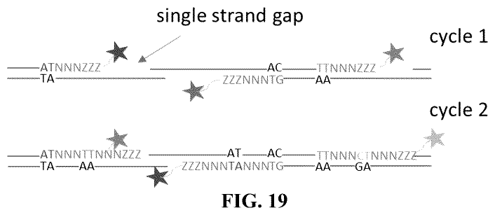

This set of 162 sgRNAs was synthesized in a single-tube reaction and used to label RR722 chromosomal DNA. The resulting samples were run on the optical mapping setup described in the methods section. Total 0.5 Gb data with an average molecule length of 244 kb was collected. FIGS. 14A-14B shows a subset of single molecules (thin lines) with good alignments to this custom-nicked reference with 100×overall coverage. As expected, no high-frequency off-target labels (>30%) were observed in this 162 set of sgRNAs. The same set of 162 sgRNAs to the RR3131 reference sequence. Only 90 perfect hits remained, and these form the RR3131 reference map shown in FIG. 15B. After aligning the labeled RR722 molecules to the RR3131 reference map, only 8 molecules aligned. These are shorter molecules around 100 kb that are aligned to two highly conserved regions, 884-981 kb of RR3131 (819-916 kb of RR722, NC_000907 and 884-981 kb of RR3131, NC_007416.02) and 1,211-1,254 kb of RR3131 (1,177-1,220 kb of RR722, NC_000907 and 1,211-1,254 kb of RR3131, NC_007416) respectively. If the normal filter of molecules longer than 150 kb were applied as shown in FIG. 14A, none of the molecules aligns to RR3131 sgRNA map. This clearly demonstrated that the custom-designed sgRNAs can uniquely identify the genomic structure of the two strains.

### EXAMPLE 8

Here it is shown for the first time that individual alleles can be differentiated at any locus across the whole genome using CRISPR-Cas9 fluorescent labeling. It could be an effective means to define long-distance haplotype structure in target regions of complex genomes, such as the human genome. This approach provides several advantages over long read sequencing techniques, including Oxford nanopore sequencing and PacBio SMRT sequencing techniques. First, the average DNA length is at 300 kb, which is more than an order longer than the read length of long-read sequencing techniques. In turn, it can span across much longer haplotype structure without computational assembly. Secondly, no target enrichment is needed to scan the whole genome to define long-distance haplotype structure in target regions, while maintaining low cost at about $500 per genome. While the target enrichment of a single region of 300 kb in the long-read sequencing target is still very challenging, as a 300 kb region counts the only 10000^{th} of the genome. A large amount of input materials are needed to generate enough starting material to create a sequencing library. Without enrichment, the cost is prohibitive to haplotype a large number of samples. Thirdly, the cost can be further reduced by generating multiple sets of sgRNAs to haplotype multiple regions.

Traditionally, genome mapping strategy is based on measuring distances between short (6-8 bp) sequence motifs across the genome, which were interrogated either by restriction enzyme cutting, or fluorescent tagging with nickase or methyltransferase (reference). However, the distribution of motifs is fixed for any given genome. Here it is also for the first time that one can customize the mapping patterns by designing a custom set of multiple sgRNAs to fluorescently tag any 20 bp sequences with CRISPR-cas9 genome editing system. This will greatly expand the applications of genome mapping in targeting specific features of interests, clinically relevant structural variants, repetitive regions, and other inaccessible regions by sequence motif labeling. More overall, one added benefit is that our multiple sgRNAs provide more sequence information than sequence motif mapping, multiple different 20mers vs the same 6-8mer. This will greatly increase the accuracy of pinpointing the breakpoints of structural variants and other specific features. The in silico mapping human genome was performed by targeting repetitive elements such as ALU and SINE-1 repeats. It was estimated that one sgRNA from ALU and one sgRNA from LINE-1 will result in 90% coverage of the human genome. This coverage is similar to the existing optical mapping schemes with Nt.Bspq1 and DLE labeling offered by Bionano Genomics. Off target hits are a lot more complicated in the human genome due to the larger genome size and long stretches of repeats.

The custom-designed genomic labeling strategies described here could find wide applications for analyzing complex genomes like humans', including determining long-range haplotype structure, higher precision breakpoint calling for complex structural variants, and improved resolution of complex repeat arrays. These strategies may also find applications in microbial comparative or community analyses since one can design gRNAs to identify characteristic markers on large genomic fragments of different microorganisms (e.g. pathogenic species) and virulence genes (e.g. antibiotic resistance genes and alleles).

Table 4. shows a set of 48 sgRNAs designed based on RR722 reference sequences. sgRNA sequences are shown below. #N/A indicates that the sgRNAs don't have a hit in RR3131. The 55 mer oligos are ordered and used in sgRNA synthesis, with the promoter sequence underlined and the overlap sequence in bold.

| | RR722 | RR3131 | |
|---|---|---|---|
| sgRNA | Locations | Locations | 55 mer oligo |
| (SEQ ID NO: 1) | 776 | 776 | (SEQ ID NO: 49) |
| GCAATCAAAGATGC | | | TTCTAATACGACTCACTATAGGCAATCAAAGATGCAGC |
| AGCGGA | | | GGAGTTTTAGAGCTAGA |
| (SEQ ID NO: 2) | 9065 | 9067 | (SEQ ID NO: 50) |
| TGTATGCACTGCAC | | | TTCTAATACGACTCACTATAGTGTATGCACTGCACAGAA |
| AGAACC | | | CCGTTTTAGAGCTAGA |
| (SEQ ID NO: 3) | 14114 | 14125 | (SEQ ID NO: 51) |
| TTTTCTTCAATATGA | | | TTCTAATACGACTCACTATAGTTTTCTTCAATATGAAGC |
| AGCCC | | | CCGTTTTAGAGCTAGA |
| (SEQ ID NO: 4) | 21722 | 21698 | (SEQ ID NO: 52) |
| GCTTTTTAGGATATC | | (off | TTCTAATACGACTCACTATAGGCTTTTTAGGATATCGTC |
| GTCCC | | target) | CCGTTTTAGAGCTAGA |
| (SEQ ID NO: 5) | 28588 | 28564 | (SEQ ID NO: 53) |
| CGAATTTCTTTATAT | | | TTCTAATACGACTCACTATAGCGAATTTCTTTATATAAG |
| AAGCG | | | CGGTTTTAGAGCTAGA |
| (SEQ ID NO: 6) | 36995 | 36973 | (SEQ ID NO: 54) |
| GGCGATGTGCTACA | | | TTCTAATACGACTCACTATAGGGCGATGTGCTACATATG |
| TATGGT | | | GTGTTTTAGAGCTAGA |
| (SEQ ID NO: 7) | 40604 | 40582 | (SEQ ID NO: 55) |
| TTACCCGTTTCTACT | | | TTCTAATACGACTCACTATAGTTACCCGTTTCTACTGCA |
| GCAGT | | | GTGTTTTAGAGCTAGA |
| (SEQ ID NO: 8) | 51392 | 52772 | (SEQ ID NO: 56) |
| ATTATTATTGTGGGA | | | TTCTAATACGACTCACTATAGATTATTATTGTGGGATTA |
| TTAAG | | | AGGTTTTAGAGCTAGA |
| (SEQ ID NO: 9) | 59529 | 60913 | (SEQ ID NO: 57) |
| GCGGTATCCACCCC | | (off | TTCTAATACGACTCACTATAGGCGGTATCCACCCCCACT |
| CACTGC | | target) | GCGTTTTAGAGCTAGA |
| (SEQ ID NO: 10) | 65581 | #N/A | (SEQ ID NO: 58) |
| TAGCCTAGGCTTAG | | | TTCTAATACGACTCACTATAGTAGCCTAGGCTTAGAGA |
| AGAGGC | | | GGCGTTTTAGAGCTAGA |
| (SEQ ID NO: 11) | 76609 | 77990 | (SEQ ID NO: 59) |
| GTGTGACATTTTGC | | | TTCTAATACGACTCACTATAGGTGTGACATTTTGCGCTA |
| GCTAAG | | | AGGTTTTAGAGCTAGA |
| (SEQ ID NO: 12) | 86065 | 86656 | (SEQ ID NO: 60) |
| GTTACATTACACACA | | (off | TTCTAATACGACTCACTATAGGTTACATTACACACAAAC |
| AACTT | | target) | TTGTTTTAGAGCTAGA |
| (SEQ ID NO: 13) | 94393 | 151264 | (SEQ ID NO: 61) |
| GGGGCGTAAATTCT | | (off | TTCTAATACGACTCACTATAGGGGGCGTAAATTCTTAAC |
| TAACAT | | target) | ATGTTTTAGAGCTAGA |
| (SEQ ID NO: 14) | 101274 | 158142 | (SEQ ID NO: 62) |
| GCATATTGTTTCACC | | | TTCTAATACGACTCACTATAGGCATATTGTTTCACCTGA |
| TGAGT | | | GTGTTTTAGAGCTAGA |
| (SEQ ID NO: 15) | 107153 | 163588 | (SEQ ID NO: 63) |
| ACAACGTCATCTCG | | | TTCTAATACGACTCACTATAGACAACGTCATCTCGGTTA |
| GTTATG | | | TGGTTTTAGAGCTAGA |
| (SEQ ID NO: 16) | 112870 | 169301 | (SEQ ID NO: 64) |
| GAATTAAAAGAACC | | | TTCTAATACGACTCACTATAGGAATTAAAAGAACCGAT |
| GATGAC | | | GACGTTTTAGAGCTAGA |
| (SEQ ID NO: 17) | 118790 | 184425 | (SEQ ID NO: 65) |
| CGTAAAGTTTTACTT | | | TTCTAATACGACTCACTATAGCGTAAAGTTTTACTTTGC |
| TGCAC | | | ACGTTTTAGAGCTAGA |
| (SEQ ID NO: 18) | 128972 | 195013 | (SEQ ID NO: 66) |
| GATCTTATAAAGAT | | | TTCTAATACGACTCACTATAGGATCTTATAAAGATAAGA |
| AAGATG | | | TGGTTTTAGAGCTAGA |
| (SEQ ID NO: 19) | 136526 | #N/A | (SEQ ID NO: 67) |
| TTTTTAATCGGCGGA | | | TTCTAATACGACTCACTATAGTTTTTAATCGGCGGAATT |
| ATTGC | | | GCGTTTTAGAGCTAGA |
| (SEQ ID NO: 20) | 141414 | 207996 | (SEQ ID NO: 68) |
| ACAACCCGCAATCTT | | | TTCTAATACGACTCACTATAGACAACCCGCAATCTTGCC |
| GCCTG | | | TGGTTTTAGAGCTAGA |
| (SEQ ID NO: 21) | 147554 | 209763 | (SEQ ID NO: 69) |
| AATATTATCGGTTG | | | TTCTAATACGACTCACTATAGAATATTATCGGTTGGTTA |
| GTTAGA | | | GAGTTTTAGAGCTAGA |
| (SEQ ID NO: 22) | 153201 | 215397 | (SEQ ID NO: 70) |
| ACTACAGGTATGAA | | | TTCTAATACGACTCACTATAGACTACAGGTATGAATCAG |
| TCAGCT | | | CTGTTTTAGAGCTAGA |
| (SEQ ID NO: 23) | 159515 | 221665 | (SEQ ID NO: 71) |
| TCTCTGATTTAGTTA | | | TTCTAATACGACTCACTATAGTCTCTGATTTAGTTAAACT |
| AACTC | | | CGTTTTAGAGCTAGA |
| (SEQ ID NO: 24) | 167020 | 229172 | (SEQ ID NO: 72) |
| TGAGAAAAAAGATT | | | TTCTAATACGACTCACTATAGTGAGAAAAAAGATTTGCT |
| TGCTAG | | | AGGTTTTAGAGCTAGA |
| (SEQ ID NO: 25) | 177007 | 239036 | (SEQ ID NO: 73) |
| GTTAAACCTACAGT | | | TTCTAATACGACTCACTATAGGTTAAACCTACAGTGCCG |
| GCCGAT | | | ATGTTTTAGAGCTAGA |
| (SEQ ID NO: 26) | 187505 | 249534 | (SEQ ID NO: 74) |
| GCTTCTCGATTTCAC | | | TTCTAATACGACTCACTATAGGCTTCTCGATTTCACCAA |
| CAACG | | | CGGTTTTAGAGCTAGA |
| (SEQ ID NO: 27) | 197054 | 259083 | (SEQ ID NO: 75) |
| TGGATAGTCGCACA | | | TTCTAATACGACTCACTATAGTGGATAGTCGCACACCTT |
| CCTTGA | | | GAGTTTTAGAGCTAGA |
| (SEQ ID NO: 28) | 202151 | 264176 | (SEQ ID NO: 76) |
| GCGAGTTTTTATGA | | | TTCTAATACGACTCACTATAGGCGAGTTTTTATGAGTAA |
| GTAATG | | | TGGTTTTAGAGCTAGA |
| (SEQ ID NO: 29) | 207665 | #N/A | (SEQ ID NO: 77) |
| GCGACGATGACGCT | | | TTCTAATACGACTCACTATAGGCGACGATGACGCTAAC |
| AACGTC | | | GTCGTTTTAGAGCTAGA |
| (SEQ ID NO: 30) | 213124 | #N/A | (SEQ ID NO: 78) |
| TCTTCAATAGGACTG | | | TTCTAATACGACTCACTATAGTCTTCAATAGGACTGAAC |
| AACCT | | | CTGTTTTAGAGCTAGA |
| (SEQ ID NO: 31) | 219206 | No label | (SEQ ID NO: 79) |
| TTGTTTTACGATATA | | | TTCTAATACGACTCACTATAGTTGTTTTACGATATAATA |
| ATACG | | | CGGTTTTAGAGCTAGA |
| (SEQ ID NO: 32) | 224792 | 286921 | (SEQ ID NO: 80) |
| TAGGTACTGTAAGA | | | TTCTAATACGACTCACTATAGTAGGTACTGTAAGAGAT |
| GATAAA | | | AAAGTTTTAGAGCTAGA |
| (SEQ ID NO: 33) | 230103 | #N/A | (SEQ ID NO: 81) |
| TAACGTATTAGATG | | | TTCTAATACGACTCACTATAGTAACGTATTAGATGCCAC |
| CCACCA | | | CAGTTTTAGAGCTAGA |
| (SEQ ID NO: 34) | 236513 | 300034 | (SEQ ID NO: 82) |
| AATGGGTCGGAAAG | | | TTCTAATACGACTCACTATAGAATGGGTCGGAAAGTAC |
| TACCGC | | | CGCGTTTTAGAGCTAGA |
| (SEQ ID NO: 35) | 248383 | 312109 | (SEQ ID NO: 83) |
| GTTAAGTTTAGTCAT | | | TTCTAATACGACTCACTATAGGTTAAGTTTAGTCATCGG |
| CGGTT | | | TTGTTTTAGAGCTAGA |
| (SEQ ID NO: 36) | 253327 | 316470 | (SEQ ID NO: 84) |
| CGAAGGGATAAATA | | (off | TTCTAATACGACTCACTATAGCGAAGGGATAAATATTG |
| TTGCGA | | target) | CGAGTTTTAGAGCTAGA |
| (SEQ ID NO: 37) | 259949 | 323064 | (SEQ ID NO: 85) |
| ATTTTCATTGTATAG | | | TTCTAATACGACTCACTATAGATTTTCATTGTATAGATG |
| ATGCG | | | CGGTTTTAGAGCTAGA |
| (SEQ ID NO: 38) | 265852 | 328965 | (SEQ ID NO: 86) |
| CAGCCGTGGAAATC | | | TTCTAATACGACTCACTATAGCAGCCGTGGAAATCCTTC |
| CTTCCG | | | CGGTTTTAGAGCTAGA |
| (SEQ ID NO: 39) | 270963 | 334078 | (SEQ ID NO: 87) |
| TAGCACTTAAAAGA | | (off | TTCTAATACGACTCACTATAGTAGCACTTAAAAGAGGA |
| GGAATG | | target) | ATGGTTTTAGAGCTAGA |
| (SEQ ID NO: 40) | 275716 | 338834 | (SEQ ID NO: 88) |
| TTACTCAAATAGTGC | | | TTCTAATACGACTCACTATAGTTACTCAAATAGTGCGTT |
| GTTAT | | | ATGTTTTAGAGCTAGA |
| (SEQ ID NO: 41) | 282039 | #N/A | (SEQ ID NO: 89) |
| GCCTGATGTGGATT | | | TTCTAATACGACTCACTATAGGCCTGATGTGGATTCTAT |
| CTATTG | | | TGGTTTTAGAGCTAGA |
| (SEQ ID NO: 42) | 289780 | 352752 | (SEQ ID NO: 90) |
| GCTCTGCCAATAATT | | | TTCTAATACGACTCACTATAGGCTCTGCCAATAATTTCT |
| TCTCA | | | CAGTTTTAGAGCTAGA |
| (SEQ ID NO: 43) | 296956 | No label | (SEQ ID NO: 91) |
| TAATCAAGCATTAG | | | TTCTAATACGACTCACTATAGTAATCAAGCATTAGATAG |
| ATAGCT | | | CTGTTTTAGAGCTAGA |
| (SEQ ID NO: 44) | 301117 | 364094 | (SEQ ID NO: 92) |
| TTTTGCATAATTCGG | | | TTCTAATACGACTCACTATAGTTTTGCATAATTCGGGGA |
| GGATC | | | TCGTTTTAGAGCTAGA |
| (SEQ ID NO: 45) | 306311 | 368783 | (SEQ ID NO: 93) |
| GCGAGTTTACTTTGA | | | TTCTAATACGACTCACTATAGGCGAGTTTACTTTGAAAT |
| AATCG | | | CGGTTTTAGAGCTAGA |
| (SEQ ID NO: 46) | 311699 | 374169 | (SEQ ID NO: 94) |
| TATTGGATGATTTTG | | | TTCTAATACGACTCACTATAGTATTGGATGATTTTGACA |
| ACACT | | | CTGTTTTAGAGCTAGA |
| (SEQ ID NO: 47) | 316712 | 379182 | (SEQ ID NO: 95) |
| ATTAAAACGAATCC | | | TTCTAATACGACTCACTATAGATTAAAACGAATCCGAGT |
| GAGTGA | | | GAGTTTTAGAGCTAGA |
| (SEQ ID NO: 48) | 322607 | #N/A | (SEQ ID NO: 96) |
| TTACTCTTGGATTAG | | | TTCTAATACGACTCACTATAGTTACTCTTGGATTAGTGG |
| TGGTA | | | TAGTTTTAGAGCTAGA |

TABLE 5
A set of 162 sOZNAs designed based on RR722 reference sequences.

| | RD | |
|---|---|---|
| sgRNA | location | 55 mer oligo |
| (SEQ ID NO: 97) | 5938 | (SEQ ID NO: 259) |
| CAAAGCGCACCACGACTG | | TTCTAATACGACTCACTATAGCAAAGCGCACCACGACTGACGTT |
| AC | | TTAGAGCTAGA |
| (SEQ ID NO: 98) | 12179 | (SEQ ID NO: 260) |
| ACTGAACCTTGCAGTACC | | TTCTAATACGACTCACTATAGACTGAACCTTGCAGTACCTCGTTT |
| TC | | TAGAGCTAGA |
| (SEQ ID NO: 99) | 19802 | (SEQ ID NO: 261) |
| TTTGTGTACTCAGCCCGA | | TTCTAATACGACTCACTATAGTTTGTGTACTCAGCCCGACCGTTT |
| CC | | TAGAGCTAGA |
| (SEQ ID NO: 100) | 26907 | (SEQ ID NO: 262) |
| AGTAGCCGTTGCAGGGA | | TTCTAATACGACTCACTATAGAGTAGCCGTTGCAGGGACACGTT |
| CAC | | TTAGAGCTAGA |
| (SEQ ID NO: 101) | 34250 | (SEQ ID NO: 263) |
| ATTGGAAAAAAACAGGC | | TTCTAATACGACTCACTATAGATTGGAAAAAAACAGGCCACGTT |
| CAC | | TTAGAGCTAGA |
| (SEQ ID NO: 102) | 50008 | (SEQ ID NO: 264) |
| GTAGTGGATACAACCTCG | | TTCTAATACGACTCACTATAGGTAGTGGATACAACCTCGGCGTT |
| GC | | TTAGAGCTAGA |
| (SEQ ID NO: 103) | 58678 | (SEQ ID NO: 265) |
| AATAAACATCACCTGTAC | | TTCTAATACGACTCACTATAGAATAAACATCACCTGTACACGTTT |
| AC | | TAGAGCTAGA |
| (SEQ ID NO: 104) | 70676 | (SEQ ID NO: 266) |
| CGCAAAAATTTTCGGCGG | | TTCTAATACGACTCACTATAGCGCAAAAATTTTCGGCGGGCGTT |
| GC | | TTAGAGCTAGA |
| (SEQ ID NO: 105) | 78546 | (SEQ ID NO: 267) |
| CAATGGCTAATTGGGCTC | | TTCTAATACGACTCACTATAGCAATGGCTAATTGGGCTCGGGTT |
| GG | | TTAGAGCTAGA |
| (SEQ ID NO: 106) | 86825 | (SEQ ID NO: 268) |
| TTTATGATAAAAGGACTC | | TTCTAATACGACTCACTATAGTTTATGATAAAAGGACTCGCGTTT |
| GC | | TAGAGCTAGA |
| (SEQ ID NO: 107) | 91668 | (SEQ ID NO: 269) |
| ATGTAGCTCGGTTCGACT | | TTCTAATACGACTCACTATAGATGTAGCTCGGTTCGACTCCGTTT |
| CC | | TAGAGCTAGA |
| (SEQ ID NO: 108) | 108151 | (SEQ ID NO: 270) |
| AGAAAGTGGGGCGGGA | | TTCTAATACGACTCACTATAGAGAAAGTGGGGCGGGAGCCTGT |
| GCCT | | TTTAGAGCTAGA |
| (SEQ ID NO: 109) | 119595 | (SEQ ID NO: 271) |
| AATACAGGTACTGCCCCG | | TTCTAATACGACTCACTATAGAATACAGGTACTGCCCCGCGGTTT |
| CG | | TAGAGCTAGA |
| (SEQ ID NO: 110) | 129036 | (SEQ ID NO: 272) |
| TAGCTCAGTTGGTAGAGC | | TTCTAATACGACTCACTATAGTAGCTCAGTTGGTAGAGCCCGTTT |
| CC | | TAGAGCTAGA |
| (SEQ ID NO: 111) | 153371 | (SEQ ID NO: 273) |
| GCACCAATTCCGCCCGCC | | TTCTAATACGACTCACTATAGGCACCAATTCCGCCCGCCCCGTTT |
| CC | | TAGAGCTAGA |
| (SEQ ID NO: 112) | 162155 | (SEQ ID NO: 274) |
| TTACAAACCAATGCCGTC | | TTCTAATACGACTCACTATAGTTACAAACCAATGCCGTCGAGTTT |
| GA | | TAGAGCTAGA |
| (SEQ ID NO: 113) | 168440 | (SEQ ID NO: 275) |
| CAAAGCAACGACCAACA | | TTCTAATACGACTCACTATAGCAAAGCAACGACCAACAGCCGTT |
| GCC | | TTAGAGCTAGA |
| (SEQ ID NO: 114) | 189456 | (SEQ ID NO: 276) |
| ATTGTAGAAGTACCGAG | | TTCTAATACGACTCACTATAGATTGTAGAAGTACCGAGAGCGTT |
| AGC | | TTAGAGCTAGA |
| (SEQ ID NO: 115) | 205901 | (SEQ ID NO: 277) |
| CGATTAATGGCAGTGGA | | TTCTAATACGACTCACTATAGCGATTAATGGCAGTGGACACGTT |
| CAC | | TTAGAGCTAGA |
| (SEQ ID NO: 116) | 223392 | (SEQ ID NO: 278) |
| ATACAATGTTGAAGCGCC | | TTCTAATACGACTCACTATAGATACAATGTTGAAGCGCCTCGTTT |
| TC | | TAGAGCTAGA |
| (SEQ ID NO: 117) | 232915 | (SEQ ID NO: 279) |
| GCGGCGATTGTTTCCTTC | | TTCTAATACGACTCACTATAGGCGGCGATTGTTTCCTTCCCGTTT |
| CC | | TAGAGCTAGA |
| (SEQ ID NO: 118) | 250043 | (SEQ ID NO: 280) |
| GCGGGTACAGAAGAGGC | | TTCTAATACGACTCACTATAGGCGGGTACAGAAGAGGCTCCGTT |
| TCC | | TTAGAGCTAGA |
| (SEQ ID NO: 119) | 258497 | (SEQ ID NO: 281) |
| GCGGCGGGTAAAATCCC | | TTCTAATACGACTCACTATAGGCGGCGGGTAAAATCCCGGGGTT |
| GGG | | TTAGAGCTAGA |
| (SEQ ID NO: 120) | 265487 | (SEQ ID NO: 282) |
| GCTTTTTGCCCCCTCCTCT | | TTCTAATACGACTCACTATAGGCTTTTTGCCCCCTCCTCTCGTTTT |
| C | | AGAGCTAGA |
| (SEQ ID NO: 121) | 274257 | (SEQ ID NO: 283) |
| TGGTTATTTTATCTTCCCC | | TTCTAATACGACTCACTATAGTGGTTATTTTATCTTCCCCGGTTTT |
| G | | AGAGCTAGA |
| (SEQ ID NO: 122) | 279533 | (SEQ ID NO: 284) |
| CCGCCGCCACTGCCTCCC | | TTCTAATACGACTCACTATAGCCGCCGCCACTGCCTCCCTCGTTT |
| TC | | TAGAGCTAGA |
| (SEQ ID NO: 123) | 285332 | (SEQ ID NO: 285) |
| TATCCAAAGGCTCTCACT | | TTCTAATACGACTCACTATAGTATCCAAAGGCTCTCACTCCGTTT |
| CC | | TAGAGCTAGA |
| (SEQ ID NO: 124) | 302615 | (SEQ ID NO: 286) |
| CAGTGAAATTAGCGGCA | | TTCTAATACGACTCACTATAGCAGTGAAATTAGCGGCAGGCGTT |
| GGC | | TTAGAGCTAGA |
| (SEQ ID NO: 125) | 313570 | (SEQ ID NO: 287) |
| GCAATACGCTCACTACGC | | TTCTAATACGACTCACTATAGGCAATACGCTCACTACGCGCGTTT |
| GC | | TAGAGCTAGA |
| (SEQ ID NO: 126) | 321776 | (SEQ ID NO: 288) |
| CGTAATATTTGACGAGAC | | TTCTAATACGACTCACTATAGCGTAATATTTGACGAGACTCGTTT |
| TC | | TAGAGCTAGA |
| (SEQ ID NO: 127) | 337549 | (SEQ ID NO: 289) |
| TTGGCGATTCTATCGGGC | | TTCTAATACGACTCACTATAGTTGGCGATTCTATCGGGCCTGTTT |
| CT | | TAGAGCTAGA |
| (SEQ ID NO: 128) | 346272 | (SEQ ID NO: 290) |
| TAACCAGTTACGCGAGA | | TTCTAATACGACTCACTATAGTAACCAGTTACGCGAGAGCCGTT |
| GCC | | TTAGAGCTAGA |
| (SEQ ID NO: 129) | 355793 | (SEQ ID NO: 291) |
| GAAATCGTCGATACAGAC | | TTCTAATACGACTCACTATAGGAAATCGTCGATACAGACCCGTTT |
| CC | | TAGAGCTAGA |
| (SEQ ID NO: 130) | 365830 | (SEQ ID NO: 292) |
| TGTATTGGGACTGGACTC | | TTCTAATACGACTCACTATAGTGTATTGGGACTGGACTCCAGTTT |
| CA | | TAGAGCTAGA |
| (SEQ ID NO: 131) | 368652 | (SEQ ID NO: 293) |
| AGTTATTTTTCCCCGATCC | | TTCTAATACGACTCACTATAGAGTTATTTTTCCCCGATCCTGTTTT |
| T | | AGAGCTAGA |
| (SEQ ID NO: 132) | 373328 | (SEQ ID NO: 294) |
| ATCTAATGCACCACTAGG | | TTCTAATACGACTCACTATAGATCTAATGCACCACTAGGACGTTT |
| AC | | TAGAGCTAGA |
| (SEQ ID NO: 133) | 392488 | (SEQ ID NO: 295) |
| TCGGAGACGAGTGCCTC | | TTCTAATACGACTCACTATAGTCGGAGACGAGTGCCTCGCCGTT |
| GCC | | TTAGAGCTAGA |
| (SEQ ID NO: 134) | 400723 | (SEQ ID NO: 296) |
| GTCAAAAGTGTTCGCGG | | TTCTAATACGACTCACTATAGGTCAAAAGTGTTCGCGGGCCGTT |
| GCC | | TTAGAGCTAGA |
| (SEQ ID NO: 135) | 423932 | (SEQ ID NO: 297) |
| TGTTCGTGCCGTGGGAG | | TTCTAATACGACTCACTATAGTGTTCGTGCCGTGGGAGGCGGTT |
| GCG | | TTAGAGCTAGA |
| (SEQ ID NO: 136) | 447969 | (SEQ ID NO: 298) |
| CCTCGCACCAAAGAGATC | | TTCTAATACGACTCACTATAGCCTCGCACCAAAGAGATCCGGTTT |
| CG | | TAGAGCTAGA |
| (SEQ ID NO: 137) | 464749 | (SEQ ID NO: 299) |
| GAAAACTTACGTTGTCTT | | TTCTAATACGACTCACTATAGGAAAACTTACGTTGTCTTCCGTTT |
| CC | | TAGAGCTAGA |
| (SEQ ID NO: 138) | 488176 | (SEQ ID NO: 300) |
| TGTTCTGGTAAAGAGACC | | TTCTAATACGACTCACTATAGTGTTCTGGTAAAGAGACCTCGTTT |
| TC | | TAGAGCTAGA |
| (SEQ ID NO: 139) | 500843 | (SEQ ID NO: 301) |
| TGTCGGTTGGTAACCTAC | | TTCTAATACGACTCACTATAGTGTCGGTTGGTAACCTACCGGTTT |
| CG | | TAGAGCTAGA |
| (SEQ ID NO: 140) | 514817 | (SEQ ID NO: 302) |
| TTTCAATTTATTGACCTCC | | TTCTAATACGACTCACTATAGTTTCAATTTATTGACCTCCGGTTTT |
| G | | AGAGCTAGA |
| (SEQ ID NO: 141) | 535877 | (SEQ ID NO: 303) |
| CCGCCATTTTATCCCCCG | | TTCTAATACGACTCACTATAGCCGCCATTTTATCCCCCGGCGTTT |
| GC | | TAGAGCTAGA |
| (SEQ ID NO: 142) | 545907 | (SEQ ID NO: 304) |
| CCAACCATTAATCCGTCT | | TTCTAATACGACTCACTATAGCCAACCATTAATCCGTCTCGGTTT |
| CG | | TAGAGCTAGA |
| (SEQ ID NO: 143) | 560347 | (SEQ ID NO: 305) |
| ATGGGAAGAAAACTGAC | | TTCTAATACGACTCACTATAGATGGGAAGAAAACTGACGGAGTT |
| GGA | | TTAGAGCTAGA |
| (SEQ ID NO: 144) | 563926 | (SEQ ID NO: 306) |
| ACTTTCCATACGGAGGGC | | TTCTAATACGACTCACTATAGACTTTCCATACGGAGGGCGCGTT |
| GC | | TTAGAGCTAGA |
| (SEQ ID NO: 145) | 571057 | (SEQ ID NO: 307) |
| TCAACTCACTGGGGGAC | | TTCTAATACGACTCACTATAGTCAACTCACTGGGGGACGGCGTT |
| GGC | | TTAGAGCTAGA |
| (SEQ ID NO: 146) | 589432 | (SEQ ID NO: 308) |
| AGCACAATGGGCTTGGA | | TTCTAATACGACTCACTATAGAGCACAATGGGCTTGGACCCGTT |
| CCC | | TTAGAGCTAGA |
| (SEQ ID NO: 147) | 592755 | (SEQ ID NO: 309) |
| AGTGACATTCCGCACTCG | | TTCTAATACGACTCACTATAGAGTGACATTCCGCACTCGTCGTTT |
| TC | | TAGAGCTAGA |
| (SEQ ID NO: 148) | 612802 | (SEQ ID NO: 310) |
| GGTGCGTTACCTTACCCT | | TTCTAATACGACTCACTATAGGGTGCGTTACCTTACCCTCCGTTT |
| CC | | TAGAGCTAGA |
| (SEQ ID NO: 149) | 617003 | (SEQ ID NO: 311) |
| TCTACACGTTGATAGGTG | | TTCTAATACGACTCACTATAGTCTACACGTTGATAGGTGCCGTTT |
| CC | | TAGAGCTAGA |
| (SEQ ID NO: 150) | 640142 | (SEQ ID NO: 312) |
| TACATTACACCAGTCCCC | | TTCTAATACGACTCACTATAGTACATTACACCAGTCCCCGGGTTT |
| GG | | TAGAGCTAGA |
| (SEQ ID NO: 151) | 644963 | (SEQ ID NO: 313) |
| TCCATTACTGGTATGGTC | | TTCTAATACGACTCACTATAGTCCATTACTGGTATGGTCCGGTTT |
| CG | | TAGAGCTAGA |
| (SEQ ID NO: 152) | 649833 | (SEQ ID NO: 314) |
| GGATTTAGAAAACGGCG | | TTCTAATACGACTCACTATAGGGATTTAGAAAACGGCGCGCGTT |
| CGC | | TTAGAGCTAGA |
| (SEQ ID NO: 153) | 662891 | (SEQ ID NO: 315) |
| GGAACCAACGCACGGAA | | TTCTAATACGACTCACTATAGGGAACCAACGCACGGAACCCGTT |
| CCC | | TTAGAGCTAGA |
| (SEQ ID NO: 154) | 683811 | (SEQ ID NO: 316) |
| CCCGCTCGTTTTGACCTA | | TTCTAATACGACTCACTATAGCCCGCTCGTTTTGACCTACGGTTT |
| CG | | TAGAGCTAGA |
| (SEQ ID NO: 155) | 686592 | (SEQ ID NO: 317) |
| GCTGATGTGTTACTCCAT | | TTCTAATACGACTCACTATAGGCTGATGTGTTACTCCATCCGTTT |
| CC | | TAGAGCTAGA |
| (SEQ ID NO: 156) | 705452 | (SEQ ID NO: 318) |
| TTTGTTACTTTTAGTCCCG | | TTCTAATACGACTCACTATAGTTTGTTACTTTTAGTCCCGTGTTTT |
| T | | AGAGCTAGA |
| (SEQ ID NO: 157) | 722377 | (SEQ ID NO: 319) |
| GCCCAAAATGCACGGACT | | TTCTAATACGACTCACTATAGGCCCAAAATGCACGGACTAGGTT |
| AG | | TTAGAGCTAGA |
| (SEQ ID NO: 158) | 728971 | (SEQ ID NO: 320) |
| GATGCGGATATTCTCGTC | | TTCTAATACGACTCACTATAGGATGCGGATATTCTCGTCCCGTTT |
| CC | | TAGAGCTAGA |
| (SEQ ID NO: 159) | 751612 | (SEQ ID NO: 321) |
| ACAAAGCTGAAAACGGC | | TTCTAATACGACTCACTATAGACAAAGCTGAAAACGGCCAGGTT |
| CAG | | TTAGAGCTAGA |
| (SEQ ID NO: 160) | 753863 | (SEQ ID NO: 322) |
| CCGGAGATGACGCCCCTC | | TTCTAATACGACTCACTATAGCCGGAGATGACGCCCCTCCGGTT |
| CG | | TTAGAGCTAGA |
| (SEQ ID NO: 161) | 764238 | (SEQ ID NO: 323) |
| CTCGAGATGTTTCAGGAG | | TTCTAATACGACTCACTATAGCTCGAGATGTTTCAGGAGAGGTT |
| AG | | TTAGAGCTAGA |
| (SEQ ID NO: 162) | 769727 | (SEQ ID NO: 324) |
| TGCAACGGTAATGACGG | | TTCTAATACGACTCACTATAGTGCAACGGTAATGACGGGGCGTT |
| GGC | | TTAGAGCTAGA |
| (SEQ ID NO: 163) | 779210 | (SEQ ID NO: 325) |
| TTTGCAGAAATTGCTCTG | | TTCTAATACGACTCACTATAGTTTGCAGAAATTGCTCTGCCGTTT |
| CC | | TAGAGCTAGA |
| (SEQ ID NO: 164) | 782680 | (SEQ ID NO: 326) |
| TTATATAACTGGCTACCG | | TTCTAATACGACTCACTATAGTTATATAACTGGCTACCGACGTTT |
| AC | | TAGAGCTAGA |
| (SEQ ID NO: 165) | 787422 | (SEQ ID NO: 327) |
| AAATCTTTGGTTCTCTCGC | | TTCTAATACGACTCACTATAGAAATCTTTGGTTCTCTCGCCGTTTT |
| C | | AGAGCTAGA |
| (SEQ ID NO: 166) | 794419 | (SEQ ID NO: 328) |
| CGGTCACTTTGCGACCTC | | TTCTAATACGACTCACTATAGCGGTCACTTTGCGACCTCAGGTTT |
| AG | | TAGAGCTAGA |
| (SEQ ID NO: 167) | 797696 | (SEQ ID NO: 329) |
| TGGTAGCATTGTTCCGTC | | TTCTAATACGACTCACTATAGTGGTAGCATTGTTCCGTCCGGTTT |
| CG | | TAGAGCTAGA |
| (SEQ ID NO: 168) | 815035 | (SEQ ID NO: 330) |
| GAGATCAAATGGTGGGT | | TTCTAATACGACTCACTATAGGAGATCAAATGGTGGGTCCTGTT |
| CCT | | TTAGAGCTAGA |
| (SEQ ID NO: 169) | 828281 | (SEQ ID NO: 331) |
| GGTGGCGTACTTACTCGC | | TTCTAATACGACTCACTATAGGGTGGCGTACTTACTCGCCGGTTT |
| CG | | TAGAGCTAGA |
| (SEQ ID NO: 170) | 842168 | (SEQ ID NO: 332) |
| GCGAACCAAGTAGAGCT | | TTCTAATACGACTCACTATAGGCGAACCAAGTAGAGCTCCAGTT |
| CCA | | TTAGAGCTAGA |
| (SEQ ID NO: 171) | 849140 | (SEQ ID NO: 333) |
| GGTTCATTCATTCCGGTT | | TTCTAATACGACTCACTATAGGGTTCATTCATTCCGGTTCCGTTT |
| CC | | TAGAGCTAGA |
| (SEQ ID NO: 172) | 851265 | (SEQ ID NO: 334) |
| ATGGTTAAAGGTCCGGG | | TTCTAATACGACTCACTATAGATGGTTAAAGGTCCGGGTCCGTT |
| TCC | | TTAGAGCTAGA |
| (SEQ ID NO: 173) | 863749 | (SEQ ID NO: 335) |
| TTAAAAAATCAACTCGGA | | TTCTAATACGACTCACTATAGTTAAAAAATCAACTCGGATCGTTT |
| TC | | TAGAGCTAGA |
| (SEQ ID NO: 174) | 865933 | (SEQ ID NO: 336) |
| GCGCAACGTTGCGTACGT | | TTCTAATACGACTCACTATAGGCGCAACGTTGCGTACGTCCGTTT |
| CC | | TAGAGCTAGA |
| (SEQ ID NO: 175) | 873808 | (SEQ ID NO: 337) |
| GATTAACTTGGTGGACCC | | TTCTAATACGACTCACTATAGGATTAACTTGGTGGACCCAGGTT |
| AG | | TTAGAGCTAGA |
| (SEQ ID NO: 176) | 875705 | (SEQ ID NO: 338) |
| TGAAATCTTATCTCACTCC | | TTCTAATACGACTCACTATAGTGAAATCTTATCTCACTCCGGTTT |
| G | | TAGAGCTAGA |
| (SEQ ID NO: 177) | 900001 | (SEQ ID NO: 339) |
| CTGCATTAAAATCACGTG | | TTCTAATACGACTCACTATAGCTGCATTAAAATCACGTGTCGTTT |
| TC | | TAGAGCTAGA |
| (SEQ ID NO: 178) | 915941 | (SEQ ID NO: 340) |
| ACTTGATCCACAACCCAG | | TTCTAATACGACTCACTATAGACTTGATCCACAACCCAGTCGTTT |
| TC | | TAGAGCTAGA |
| (SEQ ID NO: 179) | 926544 | (SEQ ID NO: 341) |
| ACTTTTGTAAAAGACCGA | | TTCTAATACGACTCACTATAGACTTTTGTAAAAGACCGACCGTTT |
| CC | | TAGAGCTAGA |
| (SEQ ID NO: 180) | 933465 | (SEQ ID NO: 342) |
| GCTGCGGCAATTGTCGCC | | TTCTAATACGACTCACTATAGGCTGCGGCAATTGTCGCCGGGTT |
| GG | | TTAGAGCTAGA |
| (SEQ ID NO: 181) | 936634 | (SEQ ID NO: 343) |
| CTGAGGTTTTAACTCTCG | | TTCTAATACGACTCACTATAGCTGAGGTTTTAACTCTCGTCGTTT |
| TC | | TAGAGCTAGA |
| (SEQ ID NO: 182) | 959006 | (SEQ ID NO: 344) |
| TTACACCAATTAAGCCAC | | TTCTAATACGACTCACTATAGTTACACCAATTAAGCCACCGGTTT |
| CG | | TAGAGCTAGA |
| (SEQ ID NO: 183) | 981428 | (SEQ ID NO: 345) |
| GGAAAAATGGTCCCCCCT | | TTCTAATACGACTCACTATAGGGAAAAATGGTCCCCCCTACGTTT |
| AC | | TAGAGCTAGA |
| (SEQ ID NO: 184) | 991831 | (SEQ ID NO: 346) |
| TCGTGGTATTTCAGGCCC | | TTCTAATACGACTCACTATAGTCGTGGTATTTCAGGCCCTGGTTT |
| TG | | TAGAGCTAGA |
| (SEQ ID NO: 185) | 1015992 | (SEQ ID NO: 347) |
| TTTCCAATTCCACGACGC | | TTCTAATACGACTCACTATAGTTTCCAATTCCACGACGCGGGTTT |
| GG | | TAGAGCTAGA |
| (SEQ ID NO: 186) | 1029811 | (SEQ ID NO: 348) |
| AAAACATTCTTACCGTCT | | TTCTAATACGACTCACTATAGAAAACATTCTTACCGTCTCCGTTT |
| CC | | TAGAGCTAGA |
| (SEQ ID NO: 187) | 1033196 | (SEQ ID NO: 349) |
| AGTTCTTTTGTCGGAGGG | | TTCTAATACGACTCACTATAGAGTTCTTTTGTCGGAGGGCCGTTT |
| CC | | TAGAGCTAGA |
| (SEQ ID NO: 188) | 1047106 | (SEQ ID NO: 350) |
| TTGGGGGACAAACCCCG | | TTCTAATACGACTCACTATAGTTGGGGGACAAACCCCGGGCGTT |
| GGC | | TTAGAGCTAGA |
| (SEQ ID NO: 189) | 1077442 | (SEQ ID NO: 351) |
| TGGCTATCAGCTTCTCGG | | TTCTAATACGACTCACTATAGTGGCTATCAGCTTCTCGGCCGTTT |
| CC | | TAGAGCTAGA |
| (SEQ ID NO: 190) | 1082624 | (SEQ ID NO: 352) |
| ATACACTAGAAAGCCTAG | | TTCTAATACGACTCACTATAGATACACTAGAAAGCCTAGTCGTTT |
| TC | | TAGAGCTAGA |
| (SEQ ID NO: 191) | 1084743 | (SEQ ID NO: 353) |
| TTTGGCATAATTCCCAGC | | TTCTAATACGACTCACTATAGTTTGGCATAATTCCCAGCTCGTTT |
| TC | | TAGAGCTAGA |
| (SEQ ID NO: 192) | 1089177 | (SEQ ID NO: 354) |
| AAAGCGAAATCTGGTCAC | | TTCTAATACGACTCACTATAGAAAGCGAAATCTGGTCACCCGTTT |
| CC | | TAGAGCTAGA |
| (SEQ ID NO: 193) | 1092341 | (SEQ ID NO: 355) |
| TTAATGTTGTATTAGGGA | | TTCTAATACGACTCACTATAGTTAATGTTGTATTAGGGACGGTTT |
| CG | | TAGAGCTAGA |
| (SEQ ID NO: 194) | 1096130 | (SEQ ID NO: 356) |
| ACTCAAGCTGTTCGCCTA | | TTCTAATACGACTCACTATAGACTCAAGCTGTTCGCCTACCGTTT |
| CC | | TAGAGCTAGA |
| (SEQ ID NO: 195) | 1104243 | (SEQ ID NO: 357) |
| AACAGCACCAGTGAGGA | | TTCTAATACGACTCACTATAGAACAGCACCAGTGAGGACGCGTT |
| CGC | | TTAGAGCTAGA |
| (SEQ ID NO: 196) | 1121583 | (SEQ ID NO: 358) |
| TGAACAGCAAATGGGTA | | TTCTAATACGACTCACTATAGTGAACAGCAAATGGGTAGGGGTT |
| GGG | | TTAGAGCTAGA |
| (SEQ ID NO: 197) | 1135939 | (SEQ ID NO: 359) |
| CATCTGCAATCACGGCGC | | TTCTAATACGACTCACTATAGCATCTGCAATCACGGCGCCAGTTT |
| CA | | TAGAGCTAGA |
| (SEQ ID NO: 198) | 1148111 | (SEQ ID NO: 360) |
| TGCATATCAGTTGGGAAC | | TTCTAATACGACTCACTATAGTGCATATCAGTTGGGAACCCGTTT |
| CC | | TAGAGCTAGA |
| (SEQ ID NO: 199) | 1155797 | (SEQ ID NO: 361) |
| AAGAAGATGCAAAACGT | | TTCTAATACGACTCACTATAGAAGAAGATGCAAAACGTCCCGTT |
| CCC | | TTAGAGCTAGA |
| (SEQ ID NO: 200) | 1162635 | (SEQ ID NO: 362) |
| TTATTTCTAAAGCACCTC | | TTCTAATACGACTCACTATAGTTATTTCTAAAGCACCTCGCGTTT |
| GC | | TAGAGCTAGA |
| (SEQ ID NO: 201) | 1172132 | (SEQ ID NO: 363) |
| GGAACCTCTTGGGGGTC | | TTCTAATACGACTCACTATAGGGAACCTCTTGGGGGTCAGCGTT |
| AGC | | TTAGAGCTAGA |
| (SEQ ID NO: 202) | 1184003 | (SEQ ID NO: 364) |
| CATTGACCATTGCCGCAG | | TTCTAATACGACTCACTATAGCATTGACCATTGCCGCAGCGGTTT |
| CG | | TAGAGCTAGA |
| (SEQ ID NO: 203) | 1190116 | (SEQ ID NO: 365) |
| TCAGAAGTGAAGGGGCT | | TTCTAATACGACTCACTATAGTCAGAAGTGAAGGGGCTGCCGTT |
| GCC | | TTAGAGCTAGA |
| (SEQ ID NO: 204) | 1208406 | (SEQ ID NO: 366) |
| CTGGCTGATTTTCAGGGG | | TTCTAATACGACTCACTATAGCTGGCTGATTTTCAGGGGGCGTT |
| GC | | TTAGAGCTAGA |
| (SEQ ID NO: 205) | 1223913 | (SEQ ID NO: 367) |
| CTGGTTTACTCGGTCAGG | | TTCTAATACGACTCACTATAGCTGGTTTACTCGGTCAGGTCGTTT |
| TC | | TAGAGCTAGA |
| (SEQ ID NO: 206) | 1242702 | (SEQ ID NO: 368) |
| CGATAACAAAACGACCA | | TTCTAATACGACTCACTATAGCGATAACAAAACGACCAGTCGTT |
| GTC | | TTAGAGCTAGA |
| (SEQ ID NO: 207) | 1250893 | (SEQ ID NO: 369) |
| TCATTACAAGGGGTCGTC | | TTCTAATACGACTCACTATAGTCATTACAAGGGGTCGTCCCGTTT |
| CC | | TAGAGCTAGA |
| (SEQ ID NO: 208) | 1255835 | (SEQ ID NO: 370) |
| GAACGCGTAGCTGCTCCT | | TTCTAATACGACTCACTATAGGAACGCGTAGCTGCTCCTCTGTTT |
| CT | | TAGAGCTAGA |
| (SEQ ID NO: 209) | 1266838 | (SEQ ID NO: 371) |
| CAATATTCGTCATACTCG | | TTCTAATACGACTCACTATAGCAATATTCGTCATACTCGGGGTTT |
| GG | | TAGAGCTAGA |
| (SEQ ID NO: 210) | 1276011 | (SEQ ID NO: 372) |
| ATCGTAATAAAAACGACG | | TTCTAATACGACTCACTATAGATCGTAATAAAAACGACGCCGTTT |
| CC | | TAGAGCTAGA |
| (SEQ ID NO: 211) | 1287103 | (SEQ ID NO: 373) |
| CAAGTGATTCGAAGTATC | | TTCTAATACGACTCACTATAGCAAGTGATTCGAAGTATCCCGTTT |
| CC | | TAGAGCTAGA |
| (SEQ ID NO: 212) | 1291289 | (SEQ ID NO: 374) |
| GTATCAGCAAACTGAGTC | | TTCTAATACGACTCACTATAGGTATCAGCAAACTGAGTCCAGTTT |
| CA | | TAGAGCTAGA |
| (SEQ ID NO: 213) | 1294399 | (SEQ ID NO: 375) |
| GTTCCTATTGGACGAATC | | TTCTAATACGACTCACTATAGGTTCCTATTGGACGAATCCCGTTT |
| CC | | TAGAGCTAGA |
| (SEQ ID NO: 214) | 1297063 | (SEQ ID NO: 376) |
| GGTTACATTATTCCCGGT | | TTCTAATACGACTCACTATAGGGTTACATTATTCCCGGTCTGTTT |
| CT | | TAGAGCTAGA |
| (SEQ ID NO: 215) | 1311638 | (SEQ ID NO: 377) |
| GACGAATTCGACCAGAAC | | TTCTAATACGACTCACTATAGGACGAATTCGACCAGAACCGGTT |
| CG | | TTAGAGCTAGA |
| (SEQ ID NO: 216) | 1323307 | (SEQ ID NO: 378) |
| TTCTCTAATTCATAGGCCC | | TTCTAATACGACTCACTATAGTTCTCTAATTCATAGGCCCCGTTTT |
| C | | AGAGCTAGA |
| (SEQ ID NO: 217) | 1325880 | (SEQ ID NO: 379) |
| ATTTGCCGTGTCCTGGCC | | TTCTAATACGACTCACTATAGATTTGCCGTGTCCTGGCCCGGTTT |
| CG | | TAGAGCTAGA |
| (SEQ ID NO: 218) | 1345521 | (SEQ ID NO: 380) |
| GGATAAATATCAGACATG | | TTCTAATACGACTCACTATAGGGATAAATATCAGACATGCCGTTT |
| CC | | TAGAGCTAGA |
| (SEQ ID NO: 219) | 1350573 | (SEQ ID NO: 381) |
| GGGCAAACAATCGTCTCG | | TTCTAATACGACTCACTATAGGGGCAAACAATCGTCTCGTCGTTT |
| TC | | TAGAGCTAGA |
| (SEQ ID NO: 220) | 1354718 | (SEQ ID NO: 382) |
| ATCGATATGCCTCCGGGC | | TTCTAATACGACTCACTATAGATCGATATGCCTCCGGGCACGTTT |
| AC | | TAGAGCTAGA |
| (SEQ ID NO: 221) | 1358727 | (SEQ ID NO: 383) |
| GGGAATTGAGTGCCAGC | | TTCTAATACGACTCACTATAGGGGAATTGAGTGCCAGCGCGGTT |
| GCG | | TTAGAGCTAGA |
| (SEQ ID NO: 222) | 1368250 | (SEQ ID NO: 384) |
| GAATGTATGGTTGCCCTG | | TTCTAATACGACTCACTATAGGAATGTATGGTTGCCCTGCCGTTT |
| CC | | TAGAGCTAGA |
| (SEQ ID NO: 223) | 1383355 | (SEQ ID NO: 385) |
| ATCACTATCGTGCGTACC | | TTCTAATACGACTCACTATAGATCACTATCGTGCGTACCCCGTTT |
| CC | | TAGAGCTAGA |
| (SEQ ID NO: 224) | 1407888 | (SEQ ID NO: 386) |
| GTGCCTAATTGAAAGGA | | TTCTAATACGACTCACTATAGGTGCCTAATTGAAAGGAGGCGTT |
| GGC | | TTAGAGCTAGA |
| (SEQ ID NO: 225) | 1437776 | (SEQ ID NO: 387) |
| GTGATTTTAGATTGGGTG | | TTCTAATACGACTCACTATAGGTGATTTTAGATTGGGTGCCGTTT |
| CC | | TAGAGCTAGA |
| (SEQ ID NO: 226) | 1453682 | (SEQ ID NO: 388) |
| AGGCATTGGATTCGGGC | | TTCTAATACGACTCACTATAGAGGCATTGGATTCGGGCCAGGTT |
| CAG | | TTAGAGCTAGA |
| (SEQ ID NO: 227) | 1463301 | (SEQ ID NO: 389) |
| AATACGTGTTCTGGAAAC | | TTCTAATACGACTCACTATAGAATACGTGTTCTGGAAACCCGTTT |
| CC | | TAGAGCTAGA |
| (SEQ ID NO: 228) | 1485658 | (SEQ ID NO: 390) |
| GTTTTTAAAGCGGCACGG | | TTCTAATACGACTCACTATAGGTTTTTAAAGCGGCACGGACGTT |
| AC | | TTAGAGCTAGA |
| (SEQ ID NO: 229) | 1498821 | (SEQ ID NO: 391) |
| AACATAAAGAGAAAGAC | | TTCTAATACGACTCACTATAGAACATAAAGAGAAAGACCCTGTT |
| CCT | | TTAGAGCTAGA |
| (SEQ ID NO: 230) | 1509314 | (SEQ ID NO: 392) |
| AAGCCGAACCATTCGAG | | TTCTAATACGACTCACTATAGAAGCCGAACCATTCGAGGCGGTT |
| GCG | | TTAGAGCTAGA |
| (SEQ ID NO: 231) | 1530862 | (SEQ ID NO: 393) |
| GTATTTATCAAACCGGGC | | TTCTAATACGACTCACTATAGGTATTTATCAAACCGGGCAGGTTT |
| AG | | TAGAGCTAGA |
| (SEQ ID NO: 232) | 1555782 | (SEQ ID NO: 394) |
| AATGAATAAAGCGCTCTC | | TTCTAATACGACTCACTATAGAATGAATAAAGCGCTCTCCGGTTT |
| CG | | TAGAGCTAGA |
| (SEQ ID NO: 233) | 1563041 | (SEQ ID NO: 395) |
| ACTCAGCAATTACGCCCC | | TTCTAATACGACTCACTATAGACTCAGCAATTACGCCCCGGGTTT |
| GG | | TAGAGCTAGA |
| (SEQ ID NO: 234) | 1572190 | (SEQ ID NO: 396) |
| CCCGTGAAGTGGCAGAG | | TTCTAATACGACTCACTATAGCCCGTGAAGTGGCAGAGGTCGTT |
| GTC | | TTAGAGCTAGA |
| (SEQ ID NO: 235) | 1578994 | (SEQ ID NO: 397) |
| CCAATCCATTCTGTCAGC | | TTCTAATACGACTCACTATAGCCAATCCATTCTGTCAGCCCGTTT |
| CC | | TAGAGCTAGA |
| (SEQ ID NO: 236) | 1582385 | (SEQ ID NO: 398) |
| CACCGAGTATGTCAGACC | | TTCTAATACGACTCACTATAGCACCGAGTATGTCAGACCGCGTT |
| GC | | TTAGAGCTAGA |
| (SEQ ID NO: 237) | 1594993 | (SEQ ID NO: 399) |
| TGCTTGGAAAGTTCGAGA | | TTCTAATACGACTCACTATAGTGCTTGGAAAGTTCGAGACAGTT |
| CA | | TTAGAGCTAGA |
| (SEQ ID NO: 238) | 1597029 | (SEQ ID NO: 400) |
| GAAAATGAAGAACGCGC | | TTCTAATACGACTCACTATAGGAAAATGAAGAACGCGCGGGGT |
| GGG | | TTTAGAGCTAGA |
| (SEQ ID NO: 239) | 1606859 | (SEQ ID NO: 401) |
| TAAATCTTCAAACTGCGG | | TTCTAATACGACTCACTATAGTAAATCTTCAAACTGCGGACGTTT |
| AC | | TAGAGCTAGA |
| (SEQ ID NO: 240) | 1612709 | (SEQ ID NO: 402) |
| GAAGCAAAAGCACTTCCG | | TTCTAATACGACTCACTATAGGAAGCAAAAGCACTTCCGCCGTT |
| CC | | TTAGAGCTAGA |
| (SEQ ID NO: 241) | 1634628 | (SEQ ID NO: 403) |
| TATATGAAAAATCATGTC | | TTCTAATACGACTCACTATAGTATATGAAAAATCATGTCCGGTTT |
| CG | | TAGAGCTAGA |
| (SEQ ID NO: 242) | 1653303 | (SEQ ID NO: 404) |
| GTGCTAGTGACTTCGGG | | TTCTAATACGACTCACTATAGGTGCTAGTGACTTCGGGGCCGTT |
| GCC | | TTAGAGCTAGA |
| (SEQ ID NO: 243) | 1664939 | (SEQ ID NO: 405) |
| TAGTGAATTAGATAGGGT | | TTCTAATACGACTCACTATAGTAGTGAATTAGATAGGGTACGTT |
| AC | | TTAGAGCTAGA |
| (SEQ ID NO: 244) | 1683153 | (SEQ ID NO: 406) |
| TATTGCTGGTGCAGGGG | | TTCTAATACGACTCACTATAGTATTGCTGGTGCAGGGGGGGGTT |
| GGG | | TTAGAGCTAGA |
| (SEQ ID NO: 245) | 1700535 | (SEQ ID NO: 407) |
| CAATTGTGCCACCACGTC | | TTCTAATACGACTCACTATAGCAATTGTGCCACCACGTCCGGTTT |
| CG | | TAGAGCTAGA |
| (SEQ ID NO: 246) | 1710116 | (SEQ ID NO: 408) |
| TGGCGTAAGTGGAACGG | | TTCTAATACGACTCACTATAGTGGCGTAAGTGGAACGGGTCGTT |
| GTC | | TTAGAGCTAGA |
| (SEQ ID NO: 247) | 1714052 | (SEQ ID NO: 409) |
| TCTGCATATCTGCCCTCCC | | TTCTAATACGACTCACTATAGTCTGCATATCTGCCCTCCCTGTTTT |
| T | | AGAGCTAGA |
| (SEQ ID NO: 248) | 1722453 | (SEQ ID NO: 410) |
| CAATTGATATTCGCCCCC | | TTCTAATACGACTCACTATAGCAATTGATATTCGCCCCCCGGTTT |
| CG | | TAGAGCTAGA |
| (SEQ ID NO: 249) | 1731210 | (SEQ ID NO: 411) |
| ATTCAGCTGTGGCAGGAC | | TTCTAATACGACTCACTATAGATTCAGCTGTGGCAGGACAGGTT |
| AG | | TTAGAGCTAGA |
| (SEQ ID NO: 250) | 1746682 | (SEQ ID NO: 412) |
| AGTGCCGGATAACGTCC | | TTCTAATACGACTCACTATAGAGTGCCGGATAACGTCCGGGGTT |
| GGG | | TTAGAGCTAGA |
| (SEQ ID NO: 251) | 1764720 | (SEQ ID NO: 413) |
| TGCTGATGTTCAAGGCTC | | TTCTAATACGACTCACTATAGTGCTGATGTTCAAGGCTCCTGTTT |
| CT | | TAGAGCTAGA |
| (SEQ ID NO: 252) | 1776710 | (SEQ ID NO: 414) |
| GTCAAATCAGGTGAGCTC | | TTCTAATACGACTCACTATAGGTCAAATCAGGTGAGCTCACGTT |
| AC | | TTAGAGCTAGA |
| (SEQ ID NO: 253) | 1796447 | (SEQ ID NO: 415) |
| ACGACCATGGTTGCCGCC | | TTCTAATACGACTCACTATAGACGACCATGGTTGCCGCCCCGTTT |
| CC | | TAGAGCTAGA |
| (SEQ ID NO: 254) | 1797663 | (SEQ ID NO: 416) |
| ATGTCAAAGGTAGCCCGC | | TTCTAATACGACTCACTATAGATGTCAAAGGTAGCCCGCCGGTT |
| CG | | TTAGAGCTAGA |
| (SEQ ID NO: 255) | 1802242 | (SEQ ID NO: 417) |
| GCGACATCCGCCATAGGC | | TTCTAATACGACTCACTATAGGCGACATCCGCCATAGGCCCGTT |
| CC | | TTAGAGCTAGA |
| (SEQ ID NO: 256) | 1804365 | (SEQ ID NO: 418) |
| TTATGGGGGAGAGCGAG | | TTCTAATACGACTCACTATAGTTATGGGGGAGAGCGAGGTCGTT |
| GTC | | TTAGAGCTAGA |
| (SEQ ID NO: 257) | 1824887 | (SEQ ID NO: 419) |
| CATCAGTTACGAGGGCG | | TTCTAATACGACTCACTATAGCATCAGTTACGAGGGCGCGTGTT |
| CGT | | TTAGAGCTAGA |
| (SEQ ID NO: 258) | 1829738 | (SEQ ID NO: 420) |
| CCTTCAACTTCACCCGGG | | TTCTAATACGACTCACTATAGCCTTCAACTTCACCCGGGCGGTTT |
| CG | | TAGAGCTAGA |

The disclosures of each and every patent, patent application, and publication cited herein are hereby incorporated herein by reference in their entirety.

While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A method of immobilizing and linearizing an oligonucleotide, the method comprising:
a) providing a micropatterned substrate, the micropatterned substrate comprising:
at least one binding region having a first width; and
at least one non-binding region having a second width;
wherein the binding regions and the non-binding regions alternate across at least a portion of the substrate;
b) contacting the micropatterned substrate with a solution comprising a at least one oligonucleotide molecule, wherein one end of at least one oligonucleotide molecule attaches to the binding region of the micropatterned substrate; and
c) combing the at least one oligonucleotide molecule such that the at least one oligonucleotide molecule extends from the binding region into at least a portion of an adjacent non-binding region;
thereby immobilizing and linearizing the at least one oligonucleotide molecule.

2. A method of optically mapping DNA, the method comprising:
a) providing a micropatterned substrate, the micropatterned substrate comprising:
at least one binding region having a first width; and
at least one non-binding region having a second width;
wherein the binding regions and the non-binding regions alternate across at least a portion of the substrate;
b) contacting the micropatterned substrate with a solution comprising at least one molecule of DNA, wherein one end of at least one molecule of DNA attaches to the binding region of the micropatterned substrate; and
c) combing the at least one molecule of DNA such that the at least one molecule of DNA extends from the binding region into at least a portion of an adjacent non-binding region; and
optically mapping the at least one molecule of DNA.

3. The method of claim 1, wherein the first width is 10 to 40 μm and the second width is 10 to 170 μm.

4. The method of claim 1, wherein the combing comprises generating a receding meniscus.

5. The method of claim 1, wherein the micropatterned substrate comprises a silica wafer.

6. The method of claim 1, wherein the binding region comprises at least one selected from the group consisting of octenyl, octadecyl, docosenyl, SU-8, polymethylmethacrylate, polydimethylsiloxane, and polystyrene.

7. The method of claim 1, wherein the non-binding region comprises at least one selected from the group consisting of polyethylene glycol (PEG), polyvinylpyrrolidone, and their derivatives.

8. The method of claim 1, further comprising:
d) coating the micropatterned substrate with a hydrogel.

9. The method of claim 2, wherein optically mapping of the at least one molecule of DNA comprises:
e) contacting the at least one molecule of DNA with at least one nicking endonuclease;
f) incorporating at least one fluorescent dye-terminator into the at least one molecule of DNA;
g) staining the at least one molecule of DNA; and
h) imaging the at least one molecule of DNA.

10. The method of claim 9, wherein the nicking endonuclease is selected from the group consisting of Nt.BspQI, Nb.BbvCI, Nt.BbvCI, Nb.BssSI, Cas9 nickase.

11. The method of claim 2, wherein optically mapping the at least one molecule of DNA comprises:
e) contacting the at least one molecule of DNA with at least one guide RNA sequence complementary to at least a portion of the at least one molecule of DNA and an inactive CRISPR-Cas9; and
f) imaging the at least one molecule of DNA.

12. The method of claim 9, wherein imaging comprises fluorescence microscopy.

13. The method of claim 12, wherein imaging comprises epifluorescence or total internal reflection fluorescence microscopy (TIRF).

14. A method of on surface DNA sequencing library generation, the method comprising:
a) providing a micropatterned substrate, the micropatterned substrate comprising:
at least one binding region having a first width; and
at least one non-binding region having a second width;
wherein the binding regions and the non-binding regions alternate across at least a portion of the substrate;
b) contacting the micropatterned substrate with a solution comprising at least one molecule of DNA, the at least one molecule of DNA comprising a T7 promoter
wherein one end of at least one molecule of DNA attaches to the binding region of the micropatterned substrate;
c) combing the at least one molecule of DNA such that the at least one molecule of DNA extends from the binding region into at least a portion of an adjacent non-binding region; and
d) generating a DNA sequencing library.

15. A method of DNA sequencing library generation, the method comprising:
a) providing a micropatterned substrate, the micropatterned substrate comprising:
at least one binding region having a first width; and
at least one non-binding region having a second width;
wherein the binding regions and the non-binding regions alternate across at least a portion of the substrate;
b) contacting the micropatterned substrate with a solution comprising at least one molecule of DNA, the at least one molecule of DNA wherein one end of at least one molecule of DNA attaches to the binding region of the micropatterned substrate;
c) combing the at least one molecule of DNA such that the at least one molecule of DNA extends from the binding region into at least a portion of an adjacent non-binding region;
d) amplifying the at least one molecule of DNA using at least one isothermal amplification method, thereby forming an amplified product;
e) eluting the amplified product from the device; and
f) generating a DNA sequencing library using the eluted amplified product.

16. The method according to claim 15, wherein the isothermal amplification method is selected from the group consisting of strand displacement at nicks or PNA-displaced sites.

17. A method of on surface DNA sequencing library generation, the method comprising:
a) providing a micropatterned substrate, the micropatterned substrate comprising:
at least one binding region having a first width; and
at least one non-binding region having a second width;
wherein the binding regions and the non-binding regions alternate across at least a portion of the substrate;
b) contacting the micropatterned substrate with a solution comprising at least one molecule of DNA, the at least one molecule of DNA wherein one end of at least one molecule of DNA attaches to the binding region of the micropatterned substrate;
c) combing the at least one molecule of DNA such that the at least one molecule of DNA extends from the binding region into at least a portion of an adjacent non-binding region;
d) performing a tagmentation reaction on the at least one molecule of DNA, thereby generating at least one tagmented product;
e) amplifying the at least one tagmented product, thereby forming an amplified product; and
f) generating a DNA sequencing library using the amplified product.

18. A method of on surface DNA sequencing, the method comprising:
a) providing a micropatterned substrate, the micropatterned substrate comprising:
at least one binding region having a first width; and
at least one non-binding region having a second width;
wherein the binding regions and the non-binding regions alternate across at least a portion of the substrate;
b) contacting the micropatterned substrate with a solution comprising at least one molecule of DNA, the at least one molecule of DNA wherein one end of at least one molecule of DNA attaches to the binding region of the micropatterned substrate;
c) combing the at least one molecule of DNA such that the at least one molecule of DNA extends from the binding region into at least a portion of an adjacent non-binding region;
d) sequencing the at least one molecule of DNA.

19. The method according to claim 18, wherein sequencing the at least one molecule of DNA comprises one or more techniques selected from group consisting of:
direct DNA sequencing by DNA polymerase with reversible DNA terminators;
generating RNA from the at least one molecule of DNA using RNA polymerase and sequencing using T7 reverse transcriptase with reversible RNA terminators;
amplifying the at least one DNA molecule on the substrate, and sequencing with reversible DNA terminators, by DNA ligation reaction with DNA ligase; and sequencing-by-hybridization using fluorescently labeled short oligonucleotides.

20. The method of claim 1, wherein the method is performed in a flow cell.

21. A method for mapping a genome, wherein the method is capable of resolving a single nucleotide polymorphism (SNP), the method comprising introducing to the genome a CRISPR/Cas9 system comprising at least one single-guide RNA (sgRNA) specific for a target sequence or a plurality of target sequences across the genome, wherein the CRISPR/Cas9 system labels the target sequence, and the target sequence or genome is analyzed.

22. The method of claim 21, wherein the CRISPR/Cas system comprises Cas9 D10A.

23. The method of claim 21, wherein the labeling comprises nick labeling.

24. The method of claim 21, wherein the analyzing is by nucleotide sequencing and/or imaging.

25. The method of claim 21, wherein the genome is a human genome or a microbial genome.

26. The method of claim 21, wherein the method is capable of distinguishing a microbe from another closely-related microbe.

27. The method of claim 21, wherein the SNP is in a protospacer adjacent motif (PAM SNP) sequence.

28. The method of claim 21, wherein the at least one sgRNA targets a PAM and/or a PAM SNP.

29. The method of claim 21, wherein the method is capable of mapping a genomic region that spans a length of the entire genome.

30. A method of defining a long distance haplotype in a genome, the method comprising administering to the genome a CRISPR/Cas9 system and a plurality of single-guide RNAs (sgRNAs) specific for a plurality of loci of a genomic region or a plurality of target regions across the genome, wherein the CRISPR/Cas9 system labels the plurality of loci of the genomic region or the plurality of target regions across the genome, and the target sequence or genome is analyzed thereby defining the long distance haplotype in the genome.

31. The method of claim 30, wherein the analyzing is by nucleotide sequencing and/or imaging.

32. The method of claim 30, wherein the genome is a human genome or a microbial genome.

33. The method of claim 30, wherein the plurality of sgRNA comprises at least one sgRNA that targets a PAM or a PAM SNP.

34. The method of claim 30, wherein the method is capable of defining long distance haplotypes that span a length of the entire genome.


SEQUENCE LISTING (471 sequences)

SEQ ID NO: 1 - DNA, 20 bp, synthetic construct
  misc_feature 1..20: note = Oligo
  source 1..20: mol_type = other DNA
gcaatcaaagatgcagcgga

SEQ ID NO: 2 - DNA, 20 bp, synthetic construct
  misc_feature 1..20: note = Oligo
  source 1..20: mol_type = other DNA
tgtatgcactgcacagaacc

SEQ ID NO: 3 - DNA, 20 bp, synthetic construct
  misc_feature 1..20: note = Oligo
  source 1..20: mol_type = other DNA
ttttcttcaatatgaagccc

SEQ ID NO: 4 - DNA, 20 bp, synthetic construct
  misc_feature 1..20: note = Oligo
  source 1..20: mol_type = other DNA
gctttttaggatatcgtccc

SEQ ID NO: 5 - DNA, 20 bp, synthetic construct
  misc_feature 1..20: note = Oligo
  source 1..20: mol_type = other DNA
cgaatttctttatataagcg

SEQ ID NO: 6 - DNA, 20 bp, synthetic construct
  misc_feature 1..20: note = Oligo
  source 1..20: mol_type = other DNA
ggcgatgtgctacatatggt

SEQ ID NO: 7 - DNA, 20 bp, synthetic construct
  misc_feature 1..20: note = Oligo
  source 1..20: mol_type = other DNA
ttacccgtttctactgcagt

SEQ ID NO: 8 - DNA, 20 bp, synthetic construct
  misc_feature 1..20: note = Oligo
  source 1..20: mol_type = other DNA
attattattgtgggattaag

SEQ ID NO: 9 - DNA, 20 bp, synthetic construct
  misc_feature 1..20: note = Oligo
  source 1..20: mol_type = other DNA
gcggtatccacccccactgc

SEQ ID NO: 10 - DNA, 20 bp, synthetic construct
  misc_feature 1..20: note = Oligo
  source 1..20: mol_type = other DNA
tagcctaggcttagagaggc

SEQ ID NO: 11 - DNA, 20 bp, synthetic construct
  misc_feature 1..20: note = Oligo
  source 1..20: mol_type = other DNA
gtgtgacattttgcgctaag

SEQ ID NO: 12 - DNA, 20 bp, synthetic construct
  misc_feature 1..20: note = Oligo
  source 1..20: mol_type = other DNA
gttacattacacacaaactt

SEQ ID NO: 13 - DNA, 20 bp, synthetic construct
  misc_feature 1..20: note = Oligo
  source 1..20: mol_type = other DNA
ggggcgtaaattcttaacat

SEQ ID NO: 14 - DNA, 20 bp, synthetic construct
  misc_feature 1..20: note = Oligo
  source 1..20: mol_type = other DNA
gcatattgtttcacctgagt

SEQ ID NO: 15 - DNA, 20 bp, synthetic construct
  misc_feature 1..20: note = Oligo
  source 1..20: mol_type = other DNA
acaacgtcatctcggttatg

SEQ ID NO: 16 - DNA, 20 bp, synthetic construct
  misc_feature 1..20: note = Oligo
  source 1..20: mol_type = other DNA
gaattaaaagaaccgatgac

SEQ ID NO: 17 - DNA, 20 bp, synthetic construct
  misc_feature 1..20: note = Oligo
  source 1..20: mol_type = other DNA
cgtaaagttttactttgcac

SEQ ID NO: 18 - DNA, 20 bp, synthetic construct
  misc_feature 1..20: note = Oligo
  source 1..20: mol_type = other DNA
gatcttataaagataagatg

SEQ ID NO: 19 - DNA, 20 bp, synthetic construct
  misc_feature 1..20: note = Oligo
  source 1..20: mol_type = other DNA
tttttaatcggcggaattgc

SEQ ID NO: 20 - DNA, 20 bp, synthetic construct
  misc_feature 1..20: note = Oligo
  source 1..20: mol_type = other DNA
acaacccgcaatcttgcctg

SEQ ID NO: 21 - DNA, 20 bp, synthetic construct
  misc_feature 1..20: note = Oligo
  source 1..20: mol_type = other DNA
aatattatcggttggttaga

SEQ ID NO: 22 - DNA, 20 bp, synthetic construct
  misc_feature 1..20: note = Oligo
  source 1..20: mol_type = other DNA
actacaggtatgaatcagct

SEQ ID NO: 23 - DNA, 20 bp, synthetic construct
  misc_feature 1..20: note = Oligo
  source 1..20: mol_type = other DNA
tctctgatttagttaaactc

SEQ ID NO: 24 - DNA, 20 bp, synthetic construct
  misc_feature 1..20: note = Oligo
  source 1..20: mol_type = other DNA
tgagaaaaaagatttgctag

SEQ ID NO: 25 - DNA, 20 bp, synthetic construct
  misc_feature 1..20: note = Oligo
  source 1..20: mol_type = other DNA
gttaaacctacagtgccgat

SEQ ID NO: 26 - DNA, 20 bp, synthetic construct
  misc_feature 1..20: note = Oligo
  source 1..20: mol_type = other DNA
gcttctcgatttcaccaacg

SEQ ID NO: 27 - DNA, 20 bp, synthetic construct
  misc_feature 1..20: note = Oligo
  source 1..20: mol_type = other DNA
tggatagtcgcacaccttga

SEQ ID NO: 28 - DNA, 20 bp, synthetic construct
  misc_feature 1..20: note = Oligo
  source 1..20: mol_type = other DNA
gcgagtttttatgagtaatg

SEQ ID NO: 29 - DNA, 20 bp, synthetic construct
  misc_feature 1..20: note = Oligo
  source 1..20: mol_type = other DNA
gcgacgatgacgctaacgtc

SEQ ID NO: 30 - DNA, 20 bp, synthetic construct
  misc_feature 1..20: note = Oligo
  source 1..20: mol_type = other DNA
tcttcaataggactgaacct

SEQ ID NO: 31 - DNA, 20 bp, synthetic construct
  misc_feature 1..20: note = Oligo
  source 1..20: mol_type = other DNA
ttgttttacgatataatacg

SEQ ID NO: 32 - DNA, 20 bp, synthetic construct
  misc_feature 1..20: note = Oligo
  source 1..20: mol_type = other DNA
taggtactgtaagagataaa

SEQ ID NO: 33 - DNA, 20 bp, synthetic construct
  misc_feature 1..20: note = Oligo
  source 1..20: mol_type = other DNA
taacgtattagatgccacca

SEQ ID NO: 34 - DNA, 20 bp, synthetic construct
  misc_feature 1..20: note = Oligo
  source 1..20: mol_type = other DNA
aatgggtcggaaagtaccgc

SEQ ID NO: 35 - DNA, 20 bp, synthetic construct
  misc_feature 1..20: note = Oligo
  source 1..20: mol_type = other DNA
gttaagtttagtcatcggtt

SEQ ID NO: 36 - DNA, 20 bp, synthetic construct
  misc_feature 1..20: note = Oligo
  source 1..20: mol_type = other DNA
cgaagggataaatattgcga

SEQ ID NO: 37 - DNA, 20 bp, synthetic construct
  misc_feature 1..20: note = Oligo
  source 1..20: mol_type = other DNA
attttcattgtatagatgcg

SEQ ID NO: 38 - DNA, 20 bp, synthetic construct
  misc_feature 1..20: note = Oligo
  source 1..20: mol_type = other DNA
cagccgtggaaatccttccg

SEQ ID NO: 39 - DNA, 20 bp, synthetic construct
  misc_feature 1..20: note = Oligo
  source 1..20: mol_type = other DNA
tagcacttaaaagaggaatg

SEQ ID NO: 40 - DNA, 20 bp, synthetic construct
  misc_feature 1..20: note = Oligo
  source 1..20: mol_type = other DNA
ttactcaaatagtgcgttat

SEQ ID NO: 41 - DNA, 20 bp, synthetic construct
  misc_feature 1..20: note = Oligo
  source 1..20: mol_type = other DNA
gcctgatgtggattctattg

SEQ ID NO: 42 - DNA, 20 bp, synthetic construct
  misc_feature 1..20: note = Oligo
  source 1..20: mol_type = other DNA
gctctgccaataatttctca

SEQ ID NO: 43 - DNA, 20 bp, synthetic construct
  misc_feature 1..20: note = Oligo
  source 1..20: mol_type = other DNA
taatcaagcattagatagct

SEQ ID NO: 44 - DNA, 20 bp, synthetic construct
  misc_feature 1..20: note = Oligo
  source 1..20: mol_type = other DNA
ttttgcataattcggggatc

SEQ ID NO: 45 - DNA, 20 bp, synthetic construct
  misc_feature 1..20: note = Oligo
  source 1..20: mol_type = other DNA
gcgagtttactttgaaatcg

SEQ ID NO: 46 - DNA, 20 bp, synthetic construct
  misc_feature 1..20: note = Oligo
  source 1..20: mol_type = other DNA
tattggatgattttgacact

SEQ ID NO: 47 - DNA, 20 bp, synthetic construct
  misc_feature 1..20: note = Oligo
  source 1..20: mol_type = other DNA
attaaaacgaatccgagtga

SEQ ID NO: 48 - DNA, 20 bp, synthetic construct
  misc_feature 1..20: note = Oligo
  source 1..20: mol_type = other DNA
ttactcttggattagtggta

SEQ ID NO: 49 - DNA, 55 bp, synthetic construct
  misc_feature 1..55: note = Oligo
  source 1..55: mol_type = other DNA
ttctaatacgactcactataggcaatcaaagatgcagcggagttttagagctaga

SEQ ID NO: 50 - DNA, 55 bp, synthetic construct
  misc_feature 1..55: note = Oligo
  source 1..55: mol_type = other DNA
ttctaatacgactcactatagtgtatgcactgcacagaaccgttttagagctaga

SEQ ID NO: 51 - DNA, 55 bp, synthetic construct
  misc_feature 1..55: note = Oligo
  source 1..55: mol_type = other DNA
ttctaatacgactcactatagttttcttcaatatgaagcccgttttagagctaga

SEQ ID NO: 52 - DNA, 55 bp, synthetic construct
  misc_feature 1..55: note = Oligo
  source 1..55: mol_type = other DNA
ttctaatacgactcactataggctttttaggatatcgtcccgttttagagctaga

SEQ ID NO: 53 - DNA, 55 bp, synthetic construct
  misc_feature 1..55: note = Oligo
  source 1..55: mol_type = other DNA
ttctaatacgactcactatagcgaatttctttatataagcggttttagagctaga

SEQ ID NO: 54 - DNA, 55 bp, synthetic construct
  misc_feature 1..55: note = Oligo
  source 1..55: mol_type = other DNA
ttctaatacgactcactatagggcgatgtgctacatatggtgttttagagctaga

SEQ ID NO: 55 - DNA, 55 bp, synthetic construct
  misc_feature 1..55: note = Oligo
  source 1..55: mol_type = other DNA
ttctaatacgactcactatagttacccgtttctactgcagtgttttagagctaga

SEQ ID NO: 56 - DNA, 55 bp, synthetic construct
  misc_feature 1..55: note = Oligo
  source 1..55: mol_type = other DNA
ttctaatacgactcactatagattattattgtgggattaaggttttagagctaga

SEQ ID NO: 57 - DNA, 55 bp, synthetic construct
  misc_feature 1..55: note = Oligo
  source 1..55: mol_type = other DNA
ttctaatacgactcactataggcggtatccacccccactgcgttttagagctaga

SEQ ID NO: 58 - DNA, 55 bp, synthetic construct
  misc_feature 1..55: note = Oligo
  source 1..55: mol_type = other DNA
ttctaatacgactcactatagtagcctaggcttagagaggcgttttagagctaga

SEQ ID NO: 59 - DNA, 55 bp, synthetic construct
  misc_feature 1..55: note = Oligo
  source 1..55: mol_type = other DNA
ttctaatacgactcactataggtgtgacattttgcgctaaggttttagagctaga

SEQ ID NO: 60 - DNA, 55 bp, synthetic construct
  misc_feature 1..55: note = Oligo
  source 1..55: mol_type = other DNA
ttctaatacgactcactataggttacattacacacaaacttgttttagagctaga

SEQ ID NO: 61 - DNA, 55 bp, synthetic construct
  misc_feature 1..55: note = Oligo
  source 1..55: mol_type = other DNA
ttctaatacgactcactatagggggcgtaaattcttaacatgttttagagctaga

SEQ ID NO: 62 - DNA, 55 bp, synthetic construct
  misc_feature 1..55: note = Oligo
  source 1..55: mol_type = other DNA
ttctaatacgactcactataggcatattgtttcacctgagtgttttagagctaga

SEQ ID NO: 63 - DNA, 55 bp, synthetic construct
  misc_feature 1..55: note = Oligo
  source 1..55: mol_type = other DNA
ttctaatacgactcactatagacaacgtcatctcggttatggttttagagctaga

SEQ ID NO: 64 - DNA, 55 bp, synthetic construct
  misc_feature 1..55: note = Oligo
  source 1..55: mol_type = other DNA
ttctaatacgactcactataggaattaaaagaaccgatgacgttttagagctaga

SEQ ID NO: 65 - DNA, 55 bp, synthetic construct
  misc_feature 1..55: note = Oligo
  source 1..55: mol_type = other DNA
ttctaatacgactcactatagcgtaaagttttactttgcacgttttagagctaga

SEQ ID NO: 66 - DNA, 55 bp, synthetic construct
  misc_feature 1..55: note = Oligo
  source 1..55: mol_type = other DNA
ttctaatacgactcactataggatcttataaagataagatggttttagagctaga

SEQ ID NO: 67 - DNA, 55 bp, synthetic construct
  misc_feature 1..55: note = Oligo
  source 1..55: mol_type = other DNA
ttctaatacgactcactatagtttttaatcggcggaattgcgttttagagctaga

SEQ ID NO: 68 - DNA, 55 bp, synthetic construct
  misc_feature 1..55: note = Oligo
  source 1..55: mol_type = other DNA
ttctaatacgactcactatagacaacccgcaatcttgcctggttttagagctaga

SEQ ID NO: 69 - DNA, 55 bp, synthetic construct
  misc_feature 1..55: note = Oligo
  source 1..55: mol_type = other DNA
ttctaatacgactcactatagaatattatcggttggttagagttttagagctaga

SEQ ID NO: 70 - DNA, 55 bp, synthetic construct
  misc_feature 1..55: note = Oligo
  source 1..55: mol_type = other DNA
ttctaatacgactcactatagactacaggtatgaatcagctgttttagagctaga

SEQ ID NO: 71 - DNA, 55 bp, synthetic construct
  misc_feature 1..55: note = Oligo
  source 1..55: mol_type = other DNA
ttctaatacgactcactatagtctctgatttagttaaactcgttttagagctaga

SEQ ID NO: 72 - DNA, 55 bp, synthetic construct
  misc_feature 1..55: note = Oligo
  source 1..55: mol_type = other DNA
ttctaatacgactcactatagtgagaaaaaagatttgctaggttttagagctaga

SEQ ID NO: 73 - DNA, 55 bp, synthetic construct
  misc_feature 1..55: note = Oligo
  source 1..55: mol_type = other DNA
ttctaatacgactcactataggttaaacctacagtgccgatgttttagagctaga

SEQ ID NO: 74 - DNA, 55 bp, synthetic construct
  misc_feature 1..55: note = Oligo
  source 1..55: mol_type = other DNA
ttctaatacgactcactataggcttctcgatttcaccaacggttttagagctaga

SEQ ID NO: 75 - DNA, 55 bp, synthetic construct
  misc_feature 1..55: note = Oligo
  source 1..55: mol_type = other DNA
ttctaatacgactcactatagtggatagtcgcacaccttgagttttagagctaga

SEQ ID NO: 76 - DNA, 55 bp, synthetic construct
  misc_feature 1..55: note = Oligo
  source 1..55: mol_type = other DNA
ttctaatacgactcactataggcgagtttttatgagtaatggttttagagctaga

SEQ ID NO: 77 - DNA, 55 bp, synthetic construct
  misc_feature 1..55: note = Oligo
  source 1..55: mol_type = other DNA
ttctaatacgactcactataggcgacgatgacgctaacgtcgttttagagctaga

SEQ ID NO: 78 - DNA, 55 bp, synthetic construct
  misc_feature 1..55: note = Oligo
  source 1..55: mol_type = other DNA
ttctaatacgactcactatagtcttcaataggactgaacctgttttagagctaga

SEQ ID NO: 79 - DNA, 55 bp, synthetic construct
  misc_feature 1..55: note = Oligo
  source 1..55: mol_type = other DNA
ttctaatacgactcactatagttgttttacgatataatacggttttagagctaga

SEQ ID NO: 80 - DNA, 55 bp, synthetic construct
  misc_feature 1..55: note = Oligo
  source 1..55: mol_type = other DNA
ttctaatacgactcactatagtaggtactgtaagagataaagttttagagctaga

SEQ ID NO: 81 - DNA, 55 bp, synthetic construct
  misc_feature 1..55: note = Oligo
  source 1..55: mol_type = other DNA
ttctaatacgactcactatagtaacgtattagatgccaccagttttagagctaga

SEQ ID NO: 82 - DNA, 55 bp, synthetic construct
  misc_feature 1..55: note = Oligo
  source 1..55: mol_type = other DNA
ttctaatacgactcactatagaatgggtcggaaagtaccgcgttttagagctaga

SEQ ID NO: 83 - DNA, 55 bp, synthetic construct
  misc_feature 1..55: note = Oligo
  source 1..55: mol_type = other DNA
ttctaatacgactcactataggttaagtttagtcatcggttgttttagagctaga

SEQ ID NO: 84 - DNA, 55 bp, synthetic construct
  misc_feature 1..55: note = Oligo
  source 1..55: mol_type = other DNA
ttctaatacgactcactatagcgaagggataaatattgcgagttttagagctaga

SEQ ID NO: 85 - DNA, 55 bp, synthetic construct
  misc_feature 1..55: note = Oligo
  source 1..55: mol_type = other DNA
ttctaatacgactcactatagattttcattgtatagatgcggttttagagctaga

SEQ ID NO: 86 - DNA, 55 bp, synthetic construct
  misc_feature 1..55: note = Oligo
  source 1..55: mol_type = other DNA
ttctaatacgactcactatagcagccgtggaaatccttccggttttagagctaga

SEQ ID NO: 87 - DNA, 55 bp, synthetic construct
  misc_feature 1..55: note = Oligo
  source 1..55: mol_type = other DNA
ttctaatacgactcactatagtagcacttaaaagaggaatggttttagagctaga

SEQ ID NO: 88 - DNA, 55 bp, synthetic construct
  misc_feature 1..55: note = Oligo
  source 1..55: mol_type = other DNA
ttctaatacgactcactatagttactcaaatagtgcgttatgttttagagctaga

SEQ ID NO: 89 - DNA, 55 bp, synthetic construct
  misc_feature 1..55: note = Oligo
  source 1..55: mol_type = other DNA
ttctaatacgactcactataggcctgatgtggattctattggttttagagctaga

SEQ ID NO: 90 - DNA, 55 bp, synthetic construct
  misc_feature 1..55: note = Oligo
  source 1..55: mol_type = other DNA
ttctaatacgactcactataggctctgccaataatttctcagttttagagctaga

SEQ ID NO: 91 - DNA, 55 bp, synthetic construct
  misc_feature 1..55: note = Oligo
  source 1..55: mol_type = other DNA
ttctaatacgactcactatagtaatcaagcattagatagctgttttagagctaga

SEQ ID NO: 92 - DNA, 55 bp, synthetic construct
  misc_feature 1..55: note = Oligo
  source 1..55: mol_type = other DNA
ttctaatacgactcactatagttttgcataattcggggatcgttttagagctaga

SEQ ID NO: 93 - DNA, 55 bp, synthetic construct
  misc_feature 1..55: note = Oligo
  source 1..55: mol_type = other DNA
ttctaatacgactcactataggcgagtttactttgaaatcggttttagagctaga

SEQ ID NO: 94 - DNA, 55 bp, synthetic construct
  misc_feature 1..55: note = Oligo
  source 1..55: mol_type = other DNA
ttctaatacgactcactatagtattggatgattttgacactgttttagagctaga

SEQ ID NO: 95 - DNA, 55 bp, synthetic construct
  misc_feature 1..55: note = Oligo
  source 1..55: mol_type = other DNA
ttctaatacgactcactatagattaaaacgaatccgagtgagttttagagctaga

SEQ ID NO: 96 - DNA, 55 bp, synthetic construct
  misc_feature 1..55: note = Oligo
  source 1..55: mol_type = other DNA
ttctaatacgactcactatagttactcttggattagtggtagttttagagctaga

SEQ ID NO: 97 - DNA, 20 bp, synthetic construct
  misc_feature 1..20: note = Oligo
  source 1..20: mol_type = other DNA
caaagcgcaccacgactgac

SEQ ID NO: 98 - DNA, 20 bp, synthetic construct
  misc_feature 1..20: note = Oligo
  source 1..20: mol_type = other DNA
actgaaccttgcagtacctc

SEQ ID NO: 99 - DNA, 20 bp, synthetic construct
  misc_feature 1..20: note = Oligo
  source 1..20: mol_type = other DNA
tttgtgtactcagcccgacc

SEQ ID NO: 100 - DNA, 20 bp, synthetic construct
  misc_feature 1..20: note = Oligo
  source 1..20: mol_type = other DNA
agtagccgttgcagggacac

SEQ ID NO: 101 - DNA, 20 bp, synthetic construct
  misc_feature 1..20: note = Oligo
  source 1..20: mol_type = other DNA
attggaaaaaaacaggccac

SEQ ID NO: 102 - DNA, 20 bp, synthetic construct
  misc_feature 1..20: note = Oligo
  source 1..20: mol_type = other DNA
gtagtggatacaacctcggc

SEQ ID NO: 103 - DNA, 20 bp, synthetic construct
  misc_feature 1..20: note = Oligo
  source 1..20: mol_type = other DNA
aataaacatcacctgtacac

SEQ ID NO: 104 - DNA, 20 bp, synthetic construct
  misc_feature 1..20: note = Oligo
  source 1..20: mol_type = other DNA
cgcaaaaattttcggcgggc

SEQ ID NO: 105 - DNA, 20 bp, synthetic construct
  misc_feature 1..20: note = Oligo
  source 1..20: mol_type = other DNA
caatggctaattgggctcgg

SEQ ID NO: 106 - DNA, 20 bp, synthetic construct
  misc_feature 1..20: note = Oligo
  source 1..20: mol_type = other DNA
tttatgataaaaggactcgc

SEQ ID NO: 107 - DNA, 20 bp, synthetic construct
  misc_feature 1..20: note = Oligo
  source 1..20: mol_type = other DNA
atgtagctcggttcgactcc

SEQ ID NO: 108 - DNA, 20 bp, synthetic construct
  misc_feature 1..20: note = Oligo
  source 1..20: mol_type = other DNA
agaaagtggggcgggagcct

SEQ ID NO: 109 - DNA, 20 bp, synthetic construct
  misc_feature 1..20: note = Oligo
  source 1..20: mol_type = other DNA
aatacaggtactgccccgcg

SEQ ID NO: 110 - DNA, 20 bp, synthetic construct
  misc_feature 1..20: note = Oligo
  source 1..20: mol_type = other DNA
tagctcagttggtagagccc

SEQ ID NO: 111 - DNA, 20 bp, synthetic construct
  misc_feature 1..20: note = Oligo
  source 1..20: mol_type = other DNA
gcaccaattccgcccgcccc

SEQ ID NO: 112 - DNA, 20 bp, synthetic construct
  misc_feature 1..20: note = Oligo
  source 1..20: mol_type = other DNA
ttacaaaccaatgccgtcga

SEQ ID NO: 113 - DNA, 20 bp, synthetic construct
  misc_feature 1..20: note = Oligo
  source 1..20: mol_type = other DNA
caaagcaacgaccaacagcc

SEQ ID NO: 114 - DNA, 20 bp, synthetic construct
  misc_feature 1..20: note = Oligo
  source 1..20: mol_type = other DNA
attgtagaagtaccgagagc

SEQ ID NO: 115 - DNA, 20 bp, synthetic construct
  misc_feature 1..20: note = Oligo
  source 1..20: mol_type = other DNA
cgattaatggcagtggacac

SEQ ID NO: 116 - DNA, 20 bp, synthetic construct
  misc_feature 1..20: note = Oligo
  source 1..20: mol_type = other DNA
atacaatgttgaagcgcctc

SEQ ID NO: 117 - DNA, 20 bp, synthetic construct
  misc_feature 1..20: note = Oligo
  source 1..20: mol_type = other DNA
gcggcgattgtttccttccc

SEQ ID NO: 118 - DNA, 20 bp, synthetic construct
  misc_feature 1..20: note = Oligo
  source 1..20: mol_type = other DNA
gcgggtacagaagaggctcc

SEQ ID NO: 119 - DNA, 20 bp, synthetic construct
  misc_feature 1..20: note = Oligo
  source 1..20: mol_type = other DNA
gcggcgggtaaaatcccggg

SEQ ID NO: 120 - DNA, 20 bp, synthetic construct
  misc_feature 1..20: note = Oligo
  source 1..20: mol_type = other DNA
gctttttgccccctcctctc

SEQ ID NO: 121 - DNA, 20 bp, synthetic construct
  misc_feature 1..20: note = Oligo
  source 1..20: mol_type = other DNA
tggttattttatcttccccg

SEQ ID NO: 122 - DNA, 20 bp, synthetic construct
  misc_feature 1..20: note = Oligo
  source 1..20: mol_type = other DNA
ccgccgccactgcctccctc

SEQ ID NO: 123 - DNA, 20 bp, synthetic construct
  misc_feature 1..20: note = Oligo
  source 1..20: mol_type = other DNA
tatccaaaggctctcactcc

SEQ ID NO: 124 - DNA, 20 bp, synthetic construct
  misc_feature 1..20: note = Oligo
  source 1..20: mol_type = other DNA
cagtgaaattagcggcaggc

SEQ ID NO: 125 - DNA, 20 bp, synthetic construct
  misc_feature 1..20: note = Oligo
  source 1..20: mol_type = other DNA
gcaatacgctcactacgcgc

SEQ ID NO: 126 - DNA, 20 bp, synthetic construct
  misc_feature 1..20: note = Oligo
  source 1..20: mol_type = other DNA
cgtaatatttgacgagactc

SEQ ID NO: 127 - DNA, 20 bp, synthetic construct
  misc_feature 1..20: note = Oligo
  source 1..20: mol_type = other DNA
ttggcgattctatcgggcct

SEQ ID NO: 128 - DNA, 20 bp, synthetic construct
  misc_feature 1..20: note = Oligo
  source 1..20: mol_type = other DNA
taaccagttacgcgagagcc

SEQ ID NO: 129 - DNA, 20 bp, synthetic construct
  misc_feature 1..20: note = Oligo
  source 1..20: mol_type = other DNA
gaaatcgtcgatacagaccc

SEQ ID NO: 130 - DNA, 20 bp, synthetic construct
  misc_feature 1..20: note = Oligo
  source 1..20: mol_type = other DNA
tgtattgggactggactcca

SEQ ID NO: 131 - DNA, 20 bp, synthetic construct
  misc_feature 1..20: note = Oligo
  source 1..20: mol_type = other DNA
agttatttttccccgatcct

SEQ ID NO: 132 - DNA, 20 bp, synthetic construct
  misc_feature 1..20: note = Oligo
  source 1..20: mol_type = other DNA
atctaatgcaccactaggac

SEQ ID NO: 133 - DNA, 20 bp, synthetic construct
  misc_feature 1..20: note = Oligo
  source 1..20: mol_type = other DNA
tcggagacgagtgcctcgcc

SEQ ID NO: 134 - DNA, 20 bp, synthetic construct
  misc_feature 1..20: note = Oligo
  source 1..20: mol_type = other DNA
gtcaaaagtgttcgcgggcc

SEQ ID NO: 135 - DNA, 20 bp, synthetic construct
  misc_feature 1..20: note = Oligo
  source 1..20: mol_type = other DNA
tgttcgtgccgtgggaggcg

SEQ ID NO: 136 - DNA, 20 bp, synthetic construct
  misc_feature 1..20: note = Oligo
  source 1..20: mol_type = other DNA
cctcgcaccaaagagatccg

SEQ ID NO: 137 - DNA, 20 bp, synthetic construct
  misc_feature 1..20: note = Oligo
  source 1..20: mol_type = other DNA
gaaaacttacgttgtcttcc

SEQ ID NO: 138 - DNA, 20 bp, synthetic construct
  misc_feature 1..20: note = Oligo
  source 1..20: mol_type = other DNA
tgttctggtaaagagacctc

SEQ ID NO: 139 - DNA, 20 bp, synthetic construct
  misc_feature 1..20: note = Oligo
  source 1..20: mol_type = other DNA
tgtcggttggtaacctaccg

SEQ ID NO: 140 - DNA, 20 bp, synthetic construct
  misc_feature 1..20: note = Oligo
  source 1..20: mol_type = other DNA
tttcaatttattgacctccg

SEQ ID NO: 141 - DNA, 20 bp, synthetic construct
  misc_feature 1..20: note = Oligo
  source 1..20: mol_type = other DNA
ccgccattttatcccccggc

SEQ ID NO: 142 - DNA, 20 bp, synthetic construct
  misc_feature 1..20: note = Oligo
  source 1..20: mol_type = other DNA
ccaaccattaatccgtctcg

SEQ ID NO: 143 - DNA, 20 bp, synthetic construct
  misc_feature 1..20: note = Oligo
  source 1..20: mol_type = other DNA
atgggaagaaaactgacgga

SEQ ID NO: 144 - DNA, 20 bp, synthetic construct
  misc_feature 1..20: note = Oligo
  source 1..20: mol_type = other DNA
actttccatacggagggcgc

SEQ ID NO: 145 - DNA, 20 bp, synthetic construct
  misc_feature 1..20: note = Oligo
  source 1..20: mol_type = other DNA
tcaactcactgggggacggc

SEQ ID NO: 146 - DNA, 20 bp, synthetic construct
  misc_feature 1..20: note = Oligo
  source 1..20: mol_type = other DNA
agcacaatgggcttggaccc

SEQ ID NO: 147 - DNA, 20 bp, synthetic construct
  misc_feature 1..20: note = Oligo
  source 1..20: mol_type = other DNA
agtgacattccgcactcgtc

SEQ ID NO: 148 - DNA, 20 bp, synthetic construct
  misc_feature 1..20: note = Oligo
  source 1..20: mol_type = other DNA
ggtgcgttaccttaccctcc

SEQ ID NO: 149 - DNA, 20 bp, synthetic construct
  misc_feature 1..20: note = Oligo
  source 1..20: mol_type = other DNA
tctacacgttgataggtgcc

SEQ ID NO: 150 - DNA, 20 bp, synthetic construct
  misc_feature 1..20: note = Oligo
  source 1..20: mol_type = other DNA
tacattacaccagtccccgg

SEQ ID NO: 151 - DNA, 20 bp, synthetic construct
  misc_feature 1..20: note = Oligo
  source 1..20: mol_type = other DNA
tccattactggtatggtccg

SEQ ID NO: 152 - DNA, 20 bp, synthetic construct
  misc_feature 1..20: note = Oligo
  source 1..20: mol_type = other DNA
ggatttagaaaacggcgcgc

SEQ ID NO: 153 - DNA, 20 bp, synthetic construct
  misc_feature 1..20: note = Oligo
  source 1..20: mol_type = other DNA
ggaaccaacgcacggaaccc

SEQ ID NO: 154 - DNA, 20 bp, synthetic construct
  misc_feature 1..20: note = Oligo
  source 1..20: mol_type = other DNA
cccgctcgttttgacctacg

SEQ ID NO: 155 - DNA, 20 bp, synthetic construct
  misc_feature 1..20: note = Oligo
  source 1..20: mol_type = other DNA
gctgatgtgttactccatcc

SEQ ID NO: 156 - DNA, 20 bp, synthetic construct
  misc_feature 1..20: note = Oligo
  source 1..20: mol_type = other DNA
tttgttacttttagtcccgt

SEQ ID NO: 157 - DNA, 20 bp, synthetic construct
  misc_feature 1..20: note = Oligo
  source 1..20: mol_type = other DNA
gcccaaaatgcacggactag

SEQ ID NO: 158 - DNA, 20 bp, synthetic construct
  misc_feature 1..20: note = Oligo
  source 1..20: mol_type = other DNA
gatgcggatattctcgtccc

SEQ ID NO: 159 - DNA, 20 bp, synthetic construct
  misc_feature 1..20: note = Oligo
  source 1..20: mol_type = other DNA
acaaagctgaaaacggccag

SEQ ID NO: 160 - DNA, 20 bp, synthetic construct
  misc_feature 1..20: note = Oligo
  source 1..20: mol_type = other DNA
ccggagatgacgcccctccg

SEQ ID NO: 161 - DNA, 20 bp, synthetic construct
  misc_feature 1..20: note = Oligo
  source 1..20: mol_type = other DNA
ctcgagatgtttcaggagag

SEQ ID NO: 162 - DNA, 20 bp, synthetic construct
  misc_feature 1..20: note = Oligo
  source 1..20: mol_type = other DNA
tgcaacggtaatgacggggc

SEQ ID NO: 163 - DNA, 20 bp, synthetic construct
  misc_feature 1..20: note = Oligo
  source 1..20: mol_type = other DNA
tttgcagaaattgctctgcc

SEQ ID NO: 164 - DNA, 20 bp, synthetic construct
  misc_feature 1..20: note = Oligo
  source 1..20: mol_type = other DNA
ttatataactggctaccgac

SEQ ID NO: 165 - DNA, 20 bp, synthetic construct
  misc_feature 1..20: note = Oligo
  source 1..20: mol_type = other DNA
aaatctttggttctctcgcc

SEQ ID NO: 166 - DNA, 20 bp, synthetic construct
  misc_feature 1..20: note = Oligo
  source 1..20: mol_type = other DNA
cggtcactttgcgacctcag

SEQ ID NO: 167 - DNA, 20 bp, synthetic construct
  misc_feature 1..20: note = Oligo
  source 1..20: mol_type = other DNA
tggtagcattgttccgtccg

SEQ ID NO: 168 - DNA, 20 bp, synthetic construct
  misc_feature 1..20: note = Oligo
  source 1..20: mol_type = other DNA
gagatcaaatggtgggtcct

SEQ ID NO: 169 - DNA, 20 bp, synthetic construct
  misc_feature 1..20: note = Oligo
  source 1..20: mol_type = other DNA
ggtggcgtacttactcgccg

SEQ ID NO: 170 - DNA, 20 bp, synthetic construct
  misc_feature 1..20: note = Oligo
  source 1..20: mol_type = other DNA
gcgaaccaagtagagctcca

SEQ ID NO: 171 - DNA, 20 bp, synthetic construct
  misc_feature 1..20: note = Oligo
  source 1..20: mol_type = other DNA
ggttcattcattccggttcc

SEQ ID NO: 172 - DNA, 20 bp, synthetic construct
  misc_feature 1..20: note = Oligo
  source 1..20: mol_type = other DNA
atggttaaaggtccgggtcc

SEQ ID NO: 173 - DNA, 20 bp, synthetic construct
  misc_feature 1..20: note = Oligo
  source 1..20: mol_type = other DNA
ttaaaaaatcaactcggatc

SEQ ID NO: 174 - DNA, 20 bp, synthetic construct
  misc_feature 1..20: note = Oligo
  source 1..20: mol_type = other DNA
gcgcaacgttgcgtacgtcc

SEQ ID NO: 175 - DNA, 20 bp, synthetic construct
  misc_feature 1..20: note = Oligo
  source 1..20: mol_type = other DNA
gattaacttggtggacccag

SEQ ID NO: 176 - DNA, 20 bp, synthetic construct
  misc_feature 1..20: note = Oligo
  source 1..20: mol_type = other DNA
tgaaatcttatctcactccg

SEQ ID NO: 177 - DNA, 20 bp, synthetic construct
  misc_feature 1..20: note = Oligo
  source 1..20: mol_type = other DNA
ctgcattaaaatcacgtgtc

SEQ ID NO: 178 - DNA, 20 bp, synthetic construct
  misc_feature 1..20: note = Oligo
  source 1..20: mol_type = other DNA
acttgatccacaacccagtc

SEQ ID NO: 179 - DNA, 20 bp, synthetic construct
  misc_feature 1..20: note = Oligo
  source 1..20: mol_type = other DNA
acttttgtaaaagaccgacc

SEQ ID NO: 180 - DNA, 20 bp, synthetic construct
  misc_feature 1..20: note = Oligo
  source 1..20: mol_type = other DNA
gctgcggcaattgtcgccgg

SEQ ID NO: 181 - DNA, 20 bp, synthetic construct
  misc_feature 1..20: note = Oligo
  source 1..20: mol_type = other DNA
ctgaggttttaactctcgtc

SEQ ID NO: 182 - DNA, 20 bp, synthetic construct
  misc_feature 1..20: note = Oligo
  source 1..20: mol_type = other DNA
ttacaccaattaagccaccg

SEQ ID NO: 183 - DNA, 20 bp, synthetic construct
  misc_feature 1..20: note = Oligo
  source 1..20: mol_type = other DNA
ggaaaaatggtcccccctac

SEQ ID NO: 184 - DNA, 20 bp, synthetic construct
  misc_feature 1..20: note = Oligo
  source 1..20: mol_type = other DNA
tcgtggtatttcaggccctg

SEQ ID NO: 185 - DNA, 20 bp, synthetic construct
  misc_feature 1..20: note = Oligo
  source 1..20: mol_type = other DNA
tttccaattccacgacgcgg

SEQ ID NO: 186 - DNA, 20 bp, synthetic construct
  misc_feature 1..20: note = Oligo
  source 1..20: mol_type = other DNA
aaaacattcttaccgtctcc

SEQ ID NO: 187 - DNA, 20 bp, synthetic construct
  misc_feature 1..20: note = Oligo
  source 1..20: mol_type = other DNA
agttcttttgtcggagggcc

SEQ ID NO: 188 - DNA, 20 bp, synthetic construct
  misc_feature 1..20: note = Oligo
  source 1..20: mol_type = other DNA
ttgggggacaaaccccgggc

SEQ ID NO: 189 - DNA, 20 bp, synthetic construct
  misc_feature 1..20: note = Oligo
  source 1..20: mol_type = other DNA
tggctatcagcttctcggcc

SEQ ID NO: 190 - DNA, 20 bp, synthetic construct
  misc_feature 1..20: note = Oligo
  source 1..20: mol_type = other DNA
atacactagaaagcctagtc

SEQ ID NO: 191 - DNA, 20 bp, synthetic construct
  misc_feature 1..20: note = Oligo
  source 1..20: mol_type = other DNA
tttggcataattcccagctc

SEQ ID NO: 192 - DNA, 20 bp, synthetic construct
  misc_feature 1..20: note = Oligo
  source 1..20: mol_type = other DNA
aaagcgaaatctggtcaccc

SEQ ID NO: 193 - DNA, 20 bp, synthetic construct
  misc_feature 1..20: note = Oligo
  source 1..20: mol_type = other DNA
ttaatgttgtattagggacg

SEQ ID NO: 194 - DNA, 20 bp, synthetic construct
  misc_feature 1..20: note = Oligo
  source 1..20: mol_type = other DNA
actcaagctgttcgcctacc

SEQ ID NO: 195 - DNA, 20 bp, synthetic construct
  misc_feature 1..20: note = Oligo
  source 1..20: mol_type = other DNA
aacagcaccagtgaggacgc

SEQ ID NO: 196 - DNA, 20 bp, synthetic construct
  misc_feature 1..20: note = Oligo
  source 1..20: mol_type = other DNA
tgaacagcaaatgggtaggg

SEQ ID NO: 197 - DNA, 20 bp, synthetic construct
  misc_feature 1..20: note = Oligo
  source 1..20: mol_type = other DNA
catctgcaatcacggcgcca

SEQ ID NO: 198 - DNA, 20 bp, synthetic construct
  misc_feature 1..20: note = Oligo
  source 1..20: mol_type = other DNA
tgcatatcagttgggaaccc

SEQ ID NO: 199 - DNA, 20 bp, synthetic construct
  misc_feature 1..20: note = Oligo
  source 1..20: mol_type = other DNA
aagaagatgcaaaacgtccc

SEQ ID NO: 200 - DNA, 20 bp, synthetic construct
  misc_feature 1..20: note = Oligo
  source 1..20: mol_type = other DNA
ttatttctaaagcacctcgc

SEQ ID NO: 201 - DNA, 20 bp, synthetic construct
  misc_feature 1..20: note = Oligo
  source 1..20: mol_type = other DNA
ggaacctcttgggggtcagc

SEQ ID NO: 202 - DNA, 20 bp, synthetic construct
  misc_feature 1..20: note = Oligo
  source 1..20: mol_type = other DNA
cattgaccattgccgcagcg

SEQ ID NO: 203 - DNA, 20 bp, synthetic construct
  misc_feature 1..20: note = Oligo
  source 1..20: mol_type = other DNA
tcagaagtgaaggggctgcc

SEQ ID NO: 204 - DNA, 20 bp, synthetic construct
  misc_feature 1..20: note = Oligo
  source 1..20: mol_type = other DNA
ctggctgattttcagggggc

SEQ ID NO: 205 - DNA, 20 bp, synthetic construct
  misc_feature 1..20: note = Oligo
  source 1..20: mol_type = other DNA
ctggtttactcggtcaggtc

SEQ ID NO: 206 - DNA, 20 bp, synthetic construct
  misc_feature 1..20: note = Oligo
  source 1..20: mol_type = other DNA
cgataacaaaacgaccagtc

SEQ ID NO: 207 - DNA, 20 bp, synthetic construct
  misc_feature 1..20: note = Oligo
  source 1..20: mol_type = other DNA
tcattacaaggggtcgtccc

SEQ ID NO: 208 - DNA, 20 bp, synthetic construct
  misc_feature 1..20: note = Oligo
  source 1..20: mol_type = other DNA
gaacgcgtagctgctcctct

SEQ ID NO: 209 - DNA, 20 bp, synthetic construct
  misc_feature 1..20: note = Oligo
  source 1..20: mol_type = other DNA
caatattcgtcatactcggg

SEQ ID NO: 210 - DNA, 20 bp, synthetic construct
  misc_feature 1..20: note = Oligo
  source 1..20: mol_type = other DNA
atcgtaataaaaacgacgcc

SEQ ID NO: 211 - DNA, 20 bp, synthetic construct
  misc_feature 1..20: note = Oligo
  source 1..20: mol_type = other DNA
caagtgattcgaagtatccc

SEQ ID NO: 212 - DNA, 20 bp, synthetic construct
  misc_feature 1..20: note = Oligo
  source 1..20: mol_type = other DNA
gtatcagcaaactgagtcca

SEQ ID NO: 213 - DNA, 20 bp, synthetic construct
  misc_feature 1..20: note = Oligo
  source 1..20: mol_type = other DNA
gttcctattggacgaatccc

SEQ ID NO: 214 - DNA, 20 bp, synthetic construct
  misc_feature 1..20: note = Oligo
  source 1..20: mol_type = other DNA
ggttacattattcccggtct

SEQ ID NO: 215 - DNA, 20 bp, synthetic construct
  misc_feature 1..20: note = Oligo
  source 1..20: mol_type = other DNA
gacgaattcgaccagaaccg

SEQ ID NO: 216 - DNA, 20 bp, synthetic construct
  misc_feature 1..20: note = Oligo
  source 1..20: mol_type = other DNA
ttctctaattcataggcccc

SEQ ID NO: 217 - DNA, 20 bp, synthetic construct
  misc_feature 1..20: note = Oligo
  source 1..20: mol_type = other DNA
atttgccgtgtcctggcccg

SEQ ID NO: 218 - DNA, 20 bp, synthetic construct
  misc_feature 1..20: note = Oligo
  source 1..20: mol_type = other DNA
ggataaatatcagacatgcc

SEQ ID NO: 219 - DNA, 20 bp, synthetic construct
  misc_feature 1..20: note = Oligo
  source 1..20: mol_type = other DNA
gggcaaacaatcgtctcgtc

SEQ ID NO: 220 - DNA, 20 bp, synthetic construct
  misc_feature 1..20: note = Oligo
  source 1..20: mol_type = other DNA
atcgatatgcctccgggcac

SEQ ID NO: 221 - DNA, 20 bp, synthetic construct
  misc_feature 1..20: note = Oligo
  source 1..20: mol_type = other DNA
gggaattgagtgccagcgcg

SEQ ID NO: 222 - DNA, 20 bp, synthetic construct
  misc_feature 1..20: note = Oligo
  source 1..20: mol_type = other DNA
gaatgtatggttgccctgcc

SEQ ID NO: 223 - DNA, 20 bp, synthetic construct
  misc_feature 1..20: note = Oligo
  source 1..20: mol_type = other DNA
atcactatcgtgcgtacccc

SEQ ID NO: 224 - DNA, 20 bp, synthetic construct
  misc_feature 1..20: note = Oligo
  source 1..20: mol_type = other DNA
gtgcctaattgaaaggaggc

SEQ ID NO: 225 - DNA, 20 bp, synthetic construct
  misc_feature 1..20: note = Oligo
  source 1..20: mol_type = other DNA
gtgattttagattgggtgcc

SEQ ID NO: 226 - DNA, 20 bp, synthetic construct
  misc_feature 1..20: note = Oligo
  source 1..20: mol_type = other DNA
aggcattggattcgggccag

SEQ ID NO: 227 - DNA, 20 bp, synthetic construct
  misc_feature 1..20: note = Oligo
  source 1..20: mol_type = other DNA
aatacgtgttctggaaaccc

SEQ ID NO: 228 - DNA, 20 bp, synthetic construct
  misc_feature 1..20: note = Oligo
  source 1..20: mol_type = other DNA
gtttttaaagcggcacggac

SEQ ID NO: 229 - DNA, 20 bp, synthetic construct
  misc_feature 1..20: note = Oligo
  source 1..20: mol_type = other DNA
aacataaagagaaagaccct

SEQ ID NO: 230 - DNA, 20 bp, synthetic construct
  misc_feature 1..20: note = Oligo
  source 1..20: mol_type = other DNA
aagccgaaccattcgaggcg

SEQ ID NO: 231 - DNA, 20 bp, synthetic construct
  misc_feature 1..20: note = Oligo
  source 1..20: mol_type = other DNA
gtatttatcaaaccgggcag

SEQ ID NO: 232 - DNA, 20 bp, synthetic construct
  misc_feature 1..20: note = Oligo
  source 1..20: mol_type = other DNA
aatgaataaagcgctctccg

SEQ ID NO: 233 - DNA, 20 bp, synthetic construct
  misc_feature 1..20: note = Oligo
  source 1..20: mol_type = other DNA
actcagcaattacgccccgg

SEQ ID NO: 234 - DNA, 20 bp, synthetic construct
  misc_feature 1..20: note = Oligo
  source 1..20: mol_type = other DNA
cccgtgaagtggcagaggtc

SEQ ID NO: 235 - DNA, 20 bp, synthetic construct
  misc_feature 1..20: note = Oligo
  source 1..20: mol_type = other DNA
ccaatccattctgtcagccc

SEQ ID NO: 236 - DNA, 20 bp, synthetic construct
  misc_feature 1..20: note = Oligo
  source 1..20: mol_type = other DNA
caccgagtatgtcagaccgc

SEQ ID NO: 237 - DNA, 20 bp, synthetic construct
  misc_feature 1..20: note = Oligo
  source 1..20: mol_type = other DNA
tgcttggaaagttcgagaca

SEQ ID NO: 238 - DNA, 20 bp, synthetic construct
  misc_feature 1..20: note = Oligo
  source 1..20: mol_type = other DNA
gaaaatgaagaacgcgcggg

SEQ ID NO: 239 - DNA, 20 bp, synthetic construct
  misc_feature 1..20: note = Oligo
  source 1..20: mol_type = other DNA
taaatcttcaaactgcggac

SEQ ID NO: 240 - DNA, 20 bp, synthetic construct
  misc_feature 1..20: note = Oligo
  source 1..20: mol_type = other DNA
gaagcaaaagcacttccgcc

SEQ ID NO: 241 - DNA, 20 bp, synthetic construct
  misc_feature 1..20: note = Oligo
  source 1..20: mol_type = other DNA
tatatgaaaaatcatgtccg

SEQ ID NO: 242 - DNA, 20 bp, synthetic construct
  misc_feature 1..20: note = Oligo
  source 1..20: mol_type = other DNA
gtgctagtgacttcggggcc

SEQ ID NO: 243 - DNA, 20 bp, synthetic construct
  misc_feature 1..20: note = Oligo
  source 1..20: mol_type = other DNA
tagtgaattagatagggtac

SEQ ID NO: 244 - DNA, 20 bp, synthetic construct
  misc_feature 1..20: note = Oligo
  source 1..20: mol_type = other DNA
tattgctggtgcaggggggg

SEQ ID NO: 245 - DNA, 20 bp, synthetic construct
  misc_feature 1..20: note = Oligo
  source 1..20: mol_type = other DNA
caattgtgccaccacgtccg

SEQ ID NO: 246 - DNA, 20 bp, synthetic construct
  misc_feature 1..20: note = Oligo
  source 1..20: mol_type = other DNA
tggcgtaagtggaacgggtc

SEQ ID NO: 247 - DNA, 20 bp, synthetic construct
  misc_feature 1..20: note = Oligo
  source 1..20: mol_type = other DNA
tctgcatatctgccctccct

SEQ ID NO: 248 - DNA, 20 bp, synthetic construct
  misc_feature 1..20: note = Oligo
  source 1..20: mol_type = other DNA
caattgatattcgccccccg

SEQ ID NO: 249 - DNA, 20 bp, synthetic construct
  misc_feature 1..20: note = Oligo
  source 1..20: mol_type = other DNA
attcagctgtggcaggacag

SEQ ID NO: 250 - DNA, 20 bp, synthetic construct
  misc_feature 1..20: note = Oligo
  source 1..20: mol_type = other DNA
agtgccggataacgtccggg

SEQ ID NO: 251 - DNA, 20 bp, synthetic construct
  misc_feature 1..20: note = Oligo
  source 1..20: mol_type = other DNA
tgctgatgttcaaggctcct

SEQ ID NO: 252 - DNA, 20 bp, synthetic construct
  misc_feature 1..20: note = Oligo
  source 1..20: mol_type = other DNA
gtcaaatcaggtgagctcac

SEQ ID NO: 253 - DNA, 20 bp, synthetic construct
  misc_feature 1..20: note = Oligo
  source 1..20: mol_type = other DNA
acgaccatggttgccgcccc

SEQ ID NO: 254 - DNA, 20 bp, synthetic construct
  misc_feature 1..20: note = Oligo
  source 1..20: mol_type = other DNA
atgtcaaaggtagcccgccg

SEQ ID NO: 255 - DNA, 20 bp, synthetic construct
  misc_feature 1..20: note = Oligo
  source 1..20: mol_type = other DNA
gcgacatccgccataggccc

SEQ ID NO: 256 - DNA, 20 bp, synthetic construct
  misc_feature 1..20: note = Oligo
  source 1..20: mol_type = other DNA
ttatgggggagagcgaggtc

SEQ ID NO: 257 - DNA, 20 bp, synthetic construct
  misc_feature 1..20: note = Oligo
  source 1..20: mol_type = other DNA
catcagttacgagggcgcgt

SEQ ID NO: 258 - DNA, 20 bp, synthetic construct
  misc_feature 1..20: note = Oligo
  source 1..20: mol_type = other DNA
ccttcaacttcacccgggcg

SEQ ID NO: 259 - DNA, 55 bp, synthetic construct
  misc_feature 1..55: note = Oligo
  source 1..55: mol_type = other DNA
ttctaatacgactcactatagcaaagcgcaccacgactgacgttttagagctaga

SEQ ID NO: 260 - DNA, 55 bp, synthetic construct
  misc_feature 1..55: note = Oligo
  source 1..55: mol_type = other DNA
ttctaatacgactcactatagactgaaccttgcagtacctcgttttagagctaga

SEQ ID NO: 261 - DNA, 55 bp, synthetic construct
  misc_feature 1..55: note = Oligo
  source 1..55: mol_type = other DNA
ttctaatacgactcactatagtttgtgtactcagcccgaccgttttagagctaga

SEQ ID NO: 262 - DNA, 55 bp, synthetic construct
  misc_feature 1..55: note = Oligo
  source 1..55: mol_type = other DNA
ttctaatacgactcactatagagtagccgttgcagggacacgttttagagctaga

SEQ ID NO: 263 - DNA, 55 bp, synthetic construct
  misc_feature 1..55: note = Oligo
  source 1..55: mol_type = other DNA
ttctaatacgactcactatagattggaaaaaaacaggccacgttttagagctaga

SEQ ID NO: 264 - DNA, 55 bp, synthetic construct
  misc_feature 1..55: note = Oligo
  source 1..55: mol_type = other DNA
ttctaatacgactcactataggtagtggatacaacctcggcgttttagagctaga

SEQ ID NO: 265 - DNA, 55 bp, synthetic construct
  misc_feature 1..55: note = Oligo
  source 1..55: mol_type = other DNA
ttctaatacgactcactatagaataaacatcacctgtacacgttttagagctaga

SEQ ID NO: 266 - DNA, 55 bp, synthetic construct
  misc_feature 1..55: note = Oligo
  source 1..55: mol_type = other DNA
ttctaatacgactcactatagcgcaaaaattttcggcgggcgttttagagctaga

SEQ ID NO: 267 - DNA, 55 bp, synthetic construct
  misc_feature 1..55: note = Oligo
  source 1..55: mol_type = other DNA
ttctaatacgactcactatagcaatggctaattgggctcgggttttagagctaga

SEQ ID NO: 268 - DNA, 55 bp, synthetic construct
  misc_feature 1..55: note = Oligo
  source 1..55: mol_type = other DNA
ttctaatacgactcactatagtttatgataaaaggactcgcgttttagagctaga

SEQ ID NO: 269 - DNA, 55 bp, synthetic construct
  misc_feature 1..55: note = Oligo
  source 1..55: mol_type = other DNA
ttctaatacgactcactatagatgtagctcggttcgactccgttttagagctaga

SEQ ID NO: 270 - DNA, 55 bp, synthetic construct
  misc_feature 1..55: note = Oligo
  source 1..55: mol_type = other DNA
ttctaatacgactcactatagagaaagtggggcgggagcctgttttagagctaga

SEQ ID NO: 271 - DNA, 55 bp, synthetic construct
  misc_feature 1..55: note = Oligo
  source 1..55: mol_type = other DNA
ttctaatacgactcactatagaatacaggtactgccccgcggttttagagctaga

SEQ ID NO: 272 - DNA, 55 bp, synthetic construct
  misc_feature 1..55: note = Oligo
  source 1..55: mol_type = other DNA
ttctaatacgactcactatagtagctcagttggtagagcccgttttagagctaga

SEQ ID NO: 273 - DNA, 55 bp, synthetic construct
  misc_feature 1..55: note = Oligo
  source 1..55: mol_type = other DNA
ttctaatacgactcactataggcaccaattccgcccgccccgttttagagctaga

SEQ ID NO: 274 - DNA, 55 bp, synthetic construct
  misc_feature 1..55: note = Oligo
  source 1..55: mol_type = other DNA
ttctaatacgactcactatagttacaaaccaatgccgtcgagttttagagctaga

SEQ ID NO: 275 - DNA, 55 bp, synthetic construct
  misc_feature 1..55: note = Oligo
  source 1..55: mol_type = other DNA
ttctaatacgactcactatagcaaagcaacgaccaacagccgttttagagctaga

SEQ ID NO: 276 - DNA, 55 bp, synthetic construct
  misc_feature 1..55: note = Oligo
  source 1..55: mol_type = other DNA
ttctaatacgactcactatagattgtagaagtaccgagagcgttttagagctaga

SEQ ID NO: 277 - DNA, 55 bp, synthetic construct
  misc_feature 1..55: note = Oligo
  source 1..55: mol_type = other DNA
ttctaatacgactcactatagcgattaatggcagtggacacgttttagagctaga

SEQ ID NO: 278 - DNA, 55 bp, synthetic construct
  misc_feature 1..55: note = Oligo
  source 1..55: mol_type = other DNA
ttctaatacgactcactatagatacaatgttgaagcgcctcgttttagagctaga

SEQ ID NO: 279 - DNA, 55 bp, synthetic construct
  misc_feature 1..55: note = Oligo
  source 1..55: mol_type = other DNA
ttctaatacgactcactataggcggcgattgtttccttcccgttttagagctaga

SEQ ID NO: 280 - DNA, 55 bp, synthetic construct
  misc_feature 1..55: note = Oligo
  source 1..55: mol_type = other DNA
ttctaatacgactcactataggcgggtacagaagaggctccgttttagagctaga

SEQ ID NO: 281 - DNA, 55 bp, synthetic construct
  misc_feature 1..55: note = Oligo
  source 1..55: mol_type = other DNA
ttctaatacgactcactataggcggcgggtaaaatcccggggttttagagctaga

SEQ ID NO: 282 - DNA, 55 bp, synthetic construct
  misc_feature 1..55: note = Oligo
  source 1..55: mol_type = other DNA
ttctaatacgactcactataggctttttgccccctcctctcgttttagagctaga

SEQ ID NO: 283 - DNA, 55 bp, synthetic construct
  misc_feature 1..55: note = Oligo
  source 1..55: mol_type = other DNA
ttctaatacgactcactatagtggttattttatcttccccggttttagagctaga

SEQ ID NO: 284 - DNA, 55 bp, synthetic construct
  misc_feature 1..55: note = Oligo
  source 1..55: mol_type = other DNA
ttctaatacgactcactatagccgccgccactgcctccctcgttttagagctaga

SEQ ID NO: 285 - DNA, 55 bp, synthetic construct
  misc_feature 1..55: note = Oligo
  source 1..55: mol_type = other DNA
ttctaatacgactcactatagtatccaaaggctctcactccgttttagagctaga

SEQ ID NO: 286 - DNA, 55 bp, synthetic construct
  misc_feature 1..55: note = Oligo
  source 1..55: mol_type = other DNA
ttctaatacgactcactatagcagtgaaattagcggcaggcgttttagagctaga

SEQ ID NO: 287 - DNA, 55 bp, synthetic construct
  misc_feature 1..55: note = Oligo
  source 1..55: mol_type = other DNA
ttctaatacgactcactataggcaatacgctcactacgcgcgttttagagctaga

SEQ ID NO: 288 - DNA, 55 bp, synthetic construct
  misc_feature 1..55: note = Oligo
  source 1..55: mol_type = other DNA
ttctaatacgactcactatagcgtaatatttgacgagactcgttttagagctaga

SEQ ID NO: 289 - DNA, 55 bp, synthetic construct
  misc_feature 1..55: note = Oligo
  source 1..55: mol_type = other DNA
ttctaatacgactcactatagttggcgattctatcgggcctgttttagagctaga

SEQ ID NO: 290 - DNA, 55 bp, synthetic construct
  misc_feature 1..55: note = Oligo
  source 1..55: mol_type = other DNA
ttctaatacgactcactatagtaaccagttacgcgagagccgttttagagctaga

SEQ ID NO: 291 - DNA, 55 bp, synthetic construct
  misc_feature 1..55: note = Oligo
  source 1..55: mol_type = other DNA
ttctaatacgactcactataggaaatcgtcgatacagacccgttttagagctaga

SEQ ID NO: 292 - DNA, 55 bp, synthetic construct
  misc_feature 1..55: note = Oligo
  source 1..55: mol_type = other DNA
ttctaatacgactcactatagtgtattgggactggactccagttttagagctaga

SEQ ID NO: 293 - DNA, 55 bp, synthetic construct
  misc_feature 1..55: note = Oligo
  source 1..55: mol_type = other DNA
ttctaatacgactcactatagagttatttttccccgatcctgttttagagctaga

SEQ ID NO: 294 - DNA, 55 bp, synthetic construct
  misc_feature 1..55: note = Oligo
  source 1..55: mol_type = other DNA
ttctaatacgactcactatagatctaatgcaccactaggacgttttagagctaga

SEQ ID NO: 295 - DNA, 55 bp, synthetic construct
  misc_feature 1..55: note = Oligo
  source 1..55: mol_type = other DNA
ttctaatacgactcactatagtcggagacgagtgcctcgccgttttagagctaga

SEQ ID NO: 296 - DNA, 55 bp, synthetic construct
  misc_feature 1..55: note = Oligo
  source 1..55: mol_type = other DNA
ttctaatacgactcactataggtcaaaagtgttcgcgggccgttttagagctaga

SEQ ID NO: 297 - DNA, 55 bp, synthetic construct
  misc_feature 1..55: note = Oligo
  source 1..55: mol_type = other DNA
ttctaatacgactcactatagtgttcgtgccgtgggaggcggttttagagctaga

SEQ ID NO: 298 - DNA, 55 bp, synthetic construct
  misc_feature 1..55: note = Oligo
  source 1..55: mol_type = other DNA
ttctaatacgactcactatagcctcgcaccaaagagatccggttttagagctaga

SEQ ID NO: 299 - DNA, 55 bp, synthetic construct
  misc_feature 1..55: note = Oligo
  source 1..55: mol_type = other DNA
ttctaatacgactcactataggaaaacttacgttgtcttccgttttagagctaga

SEQ ID NO: 300 - DNA, 55 bp, synthetic construct
  misc_feature 1..55: note = Oligo
  source 1..55: mol_type = other DNA
ttctaatacgactcactatagtgttctggtaaagagacctcgttttagagctaga

SEQ ID NO: 301 - DNA, 55 bp, synthetic construct
  misc_feature 1..55: note = Oligo
  source 1..55: mol_type = other DNA
ttctaatacgactcactatagtgtcggttggtaacctaccggttttagagctaga

SEQ ID NO: 302 - DNA, 55 bp, synthetic construct
  misc_feature 1..55: note = Oligo
  source 1..55: mol_type = other DNA
ttctaatacgactcactatagtttcaatttattgacctccggttttagagctaga

SEQ ID NO: 303 - DNA, 55 bp, synthetic construct
  misc_feature 1..55: note = Oligo
  source 1..55: mol_type = other DNA
ttctaatacgactcactatagccgccattttatcccccggcgttttagagctaga

SEQ ID NO: 304 - DNA, 55 bp, synthetic construct
  misc_feature 1..55: note = Oligo
  source 1..55: mol_type = other DNA
ttctaatacgactcactatagccaaccattaatccgtctcggttttagagctaga

SEQ ID NO: 305 - DNA, 55 bp, synthetic construct
  misc_feature 1..55: note = Oligo
  source 1..55: mol_type = other DNA
ttctaatacgactcactatagatgggaagaaaactgacggagttttagagctaga

SEQ ID NO: 306 - DNA, 55 bp, synthetic construct
  misc_feature 1..55: note = Oligo
  source 1..55: mol_type = other DNA
ttctaatacgactcactatagactttccatacggagggcgcgttttagagctaga

SEQ ID NO: 307 - DNA, 55 bp, synthetic construct
  misc_feature 1..55: note = Oligo
  source 1..55: mol_type = other DNA
ttctaatacgactcactatagtcaactcactgggggacggcgttttagagctaga

SEQ ID NO: 308 - DNA, 55 bp, synthetic construct
  misc_feature 1..55: note = Oligo
  source 1..55: mol_type = other DNA
ttctaatacgactcactatagagcacaatgggcttggacccgttttagagctaga

SEQ ID NO: 309 - DNA, 55 bp, synthetic construct
  misc_feature 1..55: note = Oligo
  source 1..55: mol_type = other DNA
ttctaatacgactcactatagagtgacattccgcactcgtcgttttagagctaga

SEQ ID NO: 310 - DNA, 55 bp, synthetic construct
  misc_feature 1..55: note = Oligo
  source 1..55: mol_type = other DNA
ttctaatacgactcactatagggtgcgttaccttaccctccgttttagagctaga

SEQ ID NO: 311 - DNA, 55 bp, synthetic construct
  misc_feature 1..55: note = Oligo
  source 1..55: mol_type = other DNA
ttctaatacgactcactatagtctacacgttgataggtgccgttttagagctaga

SEQ ID NO: 312 - DNA, 55 bp, synthetic construct
  misc_feature 1..55: note = Oligo
  source 1..55: mol_type = other DNA
ttctaatacgactcactatagtacattacaccagtccccgggttttagagctaga

SEQ ID NO: 313 - DNA, 55 bp, synthetic construct
  misc_feature 1..55: note = Oligo
  source 1..55: mol_type = other DNA
ttctaatacgactcactatagtccattactggtatggtccggttttagagctaga

SEQ ID NO: 314 - DNA, 55 bp, synthetic construct
  misc_feature 1..55: note = Oligo
  source 1..55: mol_type = other DNA
ttctaatacgactcactatagggatttagaaaacggcgcgcgttttagagctaga

SEQ ID NO: 315 - DNA, 55 bp, synthetic construct
  misc_feature 1..55: note = Oligo
  source 1..55: mol_type = other DNA
ttctaatacgactcactatagggaaccaacgcacggaacccgttttagagctaga

SEQ ID NO: 316 - DNA, 55 bp, synthetic construct
  misc_feature 1..55: note = Oligo
  source 1..55: mol_type = other DNA
ttctaatacgactcactatagcccgctcgttttgacctacggttttagagctaga

SEQ ID NO: 317 - DNA, 55 bp, synthetic construct
  misc_feature 1..55: note = Oligo
  source 1..55: mol_type = other DNA
ttctaatacgactcactataggctgatgtgttactccatccgttttagagctaga

SEQ ID NO: 318 - DNA, 55 bp, synthetic construct
  misc_feature 1..55: note = Oligo
  source 1..55: mol_type = other DNA
ttctaatacgactcactatagtttgttacttttagtcccgtgttttagagctaga

SEQ ID NO: 319 - DNA, 55 bp, synthetic construct
  misc_feature 1..55: note = Oligo
  source 1..55: mol_type = other DNA
ttctaatacgactcactataggcccaaaatgcacggactaggttttagagctaga

SEQ ID NO: 320 - DNA, 55 bp, synthetic construct
  misc_feature 1..55: note = Oligo
  source 1..55: mol_type = other DNA
ttctaatacgactcactataggatgcggatattctcgtcccgttttagagctaga

SEQ ID NO: 321 - DNA, 55 bp, synthetic construct
  misc_feature 1..55: note = Oligo
  source 1..55: mol_type = other DNA
ttctaatacgactcactatagacaaagctgaaaacggccaggttttagagctaga

SEQ ID NO: 322 - DNA, 55 bp, synthetic construct
  misc_feature 1..55: note = Oligo
  source 1..55: mol_type = other DNA
ttctaatacgactcactatagccggagatgacgcccctccggttttagagctaga

SEQ ID NO: 323 - DNA, 55 bp, synthetic construct
  misc_feature 1..55: note = Oligo
  source 1..55: mol_type = other DNA
ttctaatacgactcactatagctcgagatgtttcaggagaggttttagagctaga

SEQ ID NO: 324 - DNA, 55 bp, synthetic construct
  misc_feature 1..55: note = Oligo
  source 1..55: mol_type = other DNA
ttctaatacgactcactatagtgcaacggtaatgacggggcgttttagagctaga

SEQ ID NO: 325 - DNA, 55 bp, synthetic construct
  misc_feature 1..55: note = Oligo
  source 1..55: mol_type = other DNA
ttctaatacgactcactatagtttgcagaaattgctctgccgttttagagctaga

SEQ ID NO: 326 - DNA, 55 bp, synthetic construct
  misc_feature 1..55: note = Oligo
  source 1..55: mol_type = other DNA
ttctaatacgactcactatagttatataactggctaccgacgttttagagctaga

SEQ ID NO: 327 - DNA, 55 bp, synthetic construct
  misc_feature 1..55: note = Oligo
  source 1..55: mol_type = other DNA
ttctaatacgactcactatagaaatctttggttctctcgccgttttagagctaga

SEQ ID NO: 328 - DNA, 55 bp, synthetic construct
  misc_feature 1..55: note = Oligo
  source 1..55: mol_type = other DNA
ttctaatacgactcactatagcggtcactttgcgacctcaggttttagagctaga

SEQ ID NO: 329 - DNA, 55 bp, synthetic construct
  misc_feature 1..55: note = Oligo
  source 1..55: mol_type = other DNA
ttctaatacgactcactatagtggtagcattgttccgtccggttttagagctaga

SEQ ID NO: 330 - DNA, 55 bp, synthetic construct
  misc_feature 1..55: note = Oligo
  source 1..55: mol_type = other DNA
ttctaatacgactcactataggagatcaaatggtgggtcctgttttagagctaga

SEQ ID NO: 331 - DNA, 55 bp, synthetic construct
  misc_feature 1..55: note = Oligo
  source 1..55: mol_type = other DNA
ttctaatacgactcactatagggtggcgtacttactcgccggttttagagctaga

SEQ ID NO: 332 - DNA, 55 bp, synthetic construct
  misc_feature 1..55: note = Oligo
  source 1..55: mol_type = other DNA
ttctaatacgactcactataggcgaaccaagtagagctccagttttagagctaga

SEQ ID NO: 333 - DNA, 55 bp, synthetic construct
  misc_feature 1..55: note = Oligo
  source 1..55: mol_type = other DNA
ttctaatacgactcactatagggttcattcattccggttccgttttagagctaga

SEQ ID NO: 334 - DNA, 55 bp, synthetic construct
  misc_feature 1..55: note = Oligo
  source 1..55: mol_type = other DNA
ttctaatacgactcactatagatggttaaaggtccgggtccgttttagagctaga

SEQ ID NO: 335 - DNA, 55 bp, synthetic construct
  misc_feature 1..55: note = Oligo
  source 1..55: mol_type = other DNA
ttctaatacgactcactatagttaaaaaatcaactcggatcgttttagagctaga

SEQ ID NO: 336 - DNA, 55 bp, synthetic construct
  misc_feature 1..55: note = Oligo
  source 1..55: mol_type = other DNA
ttctaatacgactcactataggcgcaacgttgcgtacgtccgttttagagctaga

SEQ ID NO: 337 - DNA, 55 bp, synthetic construct
  misc_feature 1..55: note = Oligo
  source 1..55: mol_type = other DNA
ttctaatacgactcactataggattaacttggtggacccaggttttagagctaga

SEQ ID NO: 338 - DNA, 55 bp, synthetic construct
  misc_feature 1..55: note = Oligo
  source 1..55: mol_type = other DNA
ttctaatacgactcactatagtgaaatcttatctcactccggttttagagctaga

SEQ ID NO: 339 - DNA, 55 bp, synthetic construct
  misc_feature 1..55: note = Oligo
  source 1..55: mol_type = other DNA
ttctaatacgactcactatagctgcattaaaatcacgtgtcgttttagagctaga

SEQ ID NO: 340 - DNA, 55 bp, synthetic construct
  misc_feature 1..55: note = Oligo
  source 1..55: mol_type = other DNA
ttctaatacgactcactatagacttgatccacaacccagtcgttttagagctaga

SEQ ID NO: 341 - DNA, 55 bp, synthetic construct
  misc_feature 1..55: note = Oligo
  source 1..55: mol_type = other DNA
ttctaatacgactcactatagacttttgtaaaagaccgaccgttttagagctaga

SEQ ID NO: 342 - DNA, 55 bp, synthetic construct
  misc_feature 1..55: note = Oligo
  source 1..55: mol_type = other DNA
ttctaatacgactcactataggctgcggcaattgtcgccgggttttagagctaga

SEQ ID NO: 343 - DNA, 55 bp, synthetic construct
  misc_feature 1..55: note = Oligo
  source 1..55: mol_type = other DNA
ttctaatacgactcactatagctgaggttttaactctcgtcgttttagagctaga

SEQ ID NO: 344 - DNA, 55 bp, synthetic construct
  misc_feature 1..55: note = Oligo
  source 1..55: mol_type = other DNA
ttctaatacgactcactatagttacaccaattaagccaccggttttagagctaga

SEQ ID NO: 345 - DNA, 55 bp, synthetic construct
  misc_feature 1..55: note = Oligo
  source 1..55: mol_type = other DNA
ttctaatacgactcactatagggaaaaatggtcccccctacgttttagagctaga

SEQ ID NO: 346 - DNA, 55 bp, synthetic construct
  misc_feature 1..55: note = Oligo
  source 1..55: mol_type = other DNA
ttctaatacgactcactatagtcgtggtatttcaggccctggttttagagctaga

SEQ ID NO: 347 - DNA, 55 bp, synthetic construct
  misc_feature 1..55: note = Oligo
  source 1..55: mol_type = other DNA
ttctaatacgactcactatagtttccaattccacgacgcgggttttagagctaga

SEQ ID NO: 348 - DNA, 55 bp, synthetic construct
  misc_feature 1..55: note = Oligo
  source 1..55: mol_type = other DNA
ttctaatacgactcactatagaaaacattcttaccgtctccgttttagagctaga

SEQ ID NO: 349 - DNA, 55 bp, synthetic construct
  misc_feature 1..55: note = Oligo
  source 1..55: mol_type = other DNA
ttctaatacgactcactatagagttcttttgtcggagggccgttttagagctaga

SEQ ID NO: 350 - DNA, 55 bp, synthetic construct
  misc_feature 1..55: note = Oligo
  source 1..55: mol_type = other DNA
ttctaatacgactcactatagttgggggacaaaccccgggcgttttagagctaga

SEQ ID NO: 351 - DNA, 55 bp, synthetic construct
  misc_feature 1..55: note = Oligo
  source 1..55: mol_type = other DNA
ttctaatacgactcactatagtggctatcagcttctcggccgttttagagctaga

SEQ ID NO: 352 - DNA, 55 bp, synthetic construct
  misc_feature 1..55: note = Oligo
  source 1..55: mol_type = other DNA
ttctaatacgactcactatagatacactagaaagcctagtcgttttagagctaga

SEQ ID NO: 353 - DNA, 55 bp, synthetic construct
  misc_feature 1..55: note = Oligo
  source 1..55: mol_type = other DNA
ttctaatacgactcactatagtttggcataattcccagctcgttttagagctaga

SEQ ID NO: 354 - DNA, 55 bp, synthetic construct
  misc_feature 1..55: note = Oligo
  source 1..55: mol_type = other DNA
ttctaatacgactcactatagaaagcgaaatctggtcacccgttttagagctaga

SEQ ID NO: 355 - DNA, 55 bp, synthetic construct
  misc_feature 1..55: note = Oligo
  source 1..55: mol_type = other DNA
ttctaatacgactcactatagttaatgttgtattagggacggttttagagctaga

SEQ ID NO: 356 - DNA, 55 bp, synthetic construct
  misc_feature 1..55: note = Oligo
  source 1..55: mol_type = other DNA
ttctaatacgactcactatagactcaagctgttcgcctaccgttttagagctaga

SEQ ID NO: 357 - DNA, 55 bp, synthetic construct
  misc_feature 1..55: note = Oligo
  source 1..55: mol_type = other DNA
ttctaatacgactcactatagaacagcaccagtgaggacgcgttttagagctaga

SEQ ID NO: 358 - DNA, 55 bp, synthetic construct
  misc_feature 1..55: note = Oligo
  source 1..55: mol_type = other DNA
ttctaatacgactcactatagtgaacagcaaatgggtaggggttttagagctaga

SEQ ID NO: 359 - DNA, 55 bp, synthetic construct
  misc_feature 1..55: note = Oligo
  source 1..55: mol_type = other DNA
ttctaatacgactcactatagcatctgcaatcacggcgccagttttagagctaga

SEQ ID NO: 360 - DNA, 55 bp, synthetic construct
  misc_feature 1..55: note = Oligo
  source 1..55: mol_type = other DNA
ttctaatacgactcactatagtgcatatcagttgggaacccgttttagagctaga

SEQ ID NO: 361 - DNA, 55 bp, synthetic construct
  misc_feature 1..55: note = Oligo
  source 1..55: mol_type = other DNA
ttctaatacgactcactatagaagaagatgcaaaacgtcccgttttagagctaga

SEQ ID NO: 362 - DNA, 55 bp, synthetic construct
  misc_feature 1..55: note = Oligo
  source 1..55: mol_type = other DNA
ttctaatacgactcactatagttatttctaaagcacctcgcgttttagagctaga

SEQ ID NO: 363 - DNA, 55 bp, synthetic construct
  misc_feature 1..55: note = Oligo
  source 1..55: mol_type = other DNA
ttctaatacgactcactatagggaacctcttgggggtcagcgttttagagctaga

SEQ ID NO: 364 - DNA, 55 bp, synthetic construct
  misc_feature 1..55: note = Oligo
  source 1..55: mol_type = other DNA
ttctaatacgactcactatagcattgaccattgccgcagcggttttagagctaga

SEQ ID NO: 365 - DNA, 55 bp, synthetic construct
  misc_feature 1..55: note = Oligo
  source 1..55: mol_type = other DNA
ttctaatacgactcactatagtcagaagtgaaggggctgccgttttagagctaga

SEQ ID NO: 366 - DNA, 55 bp, synthetic construct
  misc_feature 1..55: note = Oligo
  source 1..55: mol_type = other DNA
ttctaatacgactcactatagctggctgattttcagggggcgttttagagctaga

SEQ ID NO: 367 - DNA, 55 bp, synthetic construct
  misc_feature 1..55: note = Oligo
  source 1..55: mol_type = other DNA
ttctaatacgactcactatagctggtttactcggtcaggtcgttttagagctaga

SEQ ID NO: 368 - DNA, 55 bp, synthetic construct
  misc_feature 1..55: note = Oligo
  source 1..55: mol_type = other DNA
ttctaatacgactcactatagcgataacaaaacgaccagtcgttttagagctaga

SEQ ID NO: 369 - DNA, 55 bp, synthetic construct
  misc_feature 1..55: note = Oligo
  source 1..55: mol_type = other DNA
ttctaatacgactcactatagtcattacaaggggtcgtcccgttttagagctaga

SEQ ID NO: 370 - DNA, 55 bp, synthetic construct
  misc_feature 1..55: note = Oligo
  source 1..55: mol_type = other DNA
ttctaatacgactcactataggaacgcgtagctgctcctctgttttagagctaga

SEQ ID NO: 371 - DNA, 55 bp, synthetic construct
  misc_feature 1..55: note = Oligo
  source 1..55: mol_type = other DNA
ttctaatacgactcactatagcaatattcgtcatactcggggttttagagctaga

SEQ ID NO: 372 - DNA, 55 bp, synthetic construct
  misc_feature 1..55: note = Oligo
  source 1..55: mol_type = other DNA
ttctaatacgactcactatagatcgtaataaaaacgacgccgttttagagctaga

SEQ ID NO: 373 - DNA, 55 bp, synthetic construct
  misc_feature 1..55: note = Oligo
  source 1..55: mol_type = other DNA
ttctaatacgactcactatagcaagtgattcgaagtatcccgttttagagctaga

SEQ ID NO: 374 - DNA, 55 bp, synthetic construct
  misc_feature 1..55: note = Oligo
  source 1..55: mol_type = other DNA
ttctaatacgactcactataggtatcagcaaactgagtccagttttagagctaga

SEQ ID NO: 375 - DNA, 55 bp, synthetic construct
  misc_feature 1..55: note = Oligo
  source 1..55: mol_type = other DNA
ttctaatacgactcactataggttcctattggacgaatcccgttttagagctaga

SEQ ID NO: 376 - DNA, 55 bp, synthetic construct
  misc_feature 1..55: note = Oligo
  source 1..55: mol_type = other DNA
ttctaatacgactcactatagggttacattattcccggtctgttttagagctaga

SEQ ID NO: 377 - DNA, 55 bp, synthetic construct
  misc_feature 1..55: note = Oligo
  source 1..55: mol_type = other DNA
ttctaatacgactcactataggacgaattcgaccagaaccggttttagagctaga

SEQ ID NO: 378 - DNA, 55 bp, synthetic construct
  misc_feature 1..55: note = Oligo
  source 1..55: mol_type = other DNA
ttctaatacgactcactatagttctctaattcataggccccgttttagagctaga

SEQ ID NO: 379 - DNA, 55 bp, synthetic construct
  misc_feature 1..55: note = Oligo
  source 1..55: mol_type = other DNA
ttctaatacgactcactatagatttgccgtgtcctggcccggttttagagctaga

SEQ ID NO: 380 - DNA, 55 bp, synthetic construct
  misc_feature 1..55: note = Oligo
  source 1..55: mol_type = other DNA
ttctaatacgactcactatagggataaatatcagacatgccgttttagagctaga

SEQ ID NO: 381 - DNA, 55 bp, synthetic construct
  misc_feature 1..55: note = Oligo
  source 1..55: mol_type = other DNA
ttctaatacgactcactataggggcaaacaatcgtctcgtcgttttagagctaga

SEQ ID NO: 382 - DNA, 55 bp, synthetic construct
  misc_feature 1..55: note = Oligo
  source 1..55: mol_type = other DNA
ttctaatacgactcactatagatcgatatgcctccgggcacgttttagagctaga

SEQ ID NO: 383 - DNA, 55 bp, synthetic construct
  misc_feature 1..55: note = Oligo
  source 1..55: mol_type = other DNA
ttctaatacgactcactataggggaattgagtgccagcgcggttttagagctaga

SEQ ID NO: 384 - DNA, 55 bp, synthetic construct
  misc_feature 1..55: note = Oligo
  source 1..55: mol_type = other DNA
ttctaatacgactcactataggaatgtatggttgccctgccgttttagagctaga

SEQ ID NO: 385 - DNA, 55 bp, synthetic construct
  misc_feature 1..55: note = Oligo
  source 1..55: mol_type = other DNA
ttctaatacgactcactatagatcactatcgtgcgtaccccgttttagagctaga

SEQ ID NO: 386 - DNA, 55 bp, synthetic construct
  misc_feature 1..55: note = Oligo
  source 1..55: mol_type = other DNA
ttctaatacgactcactataggtgcctaattgaaaggaggcgttttagagctaga

SEQ ID NO: 387 - DNA, 55 bp, synthetic construct
  misc_feature 1..55: note = Oligo
  source 1..55: mol_type = other DNA
ttctaatacgactcactataggtgattttagattgggtgccgttttagagctaga

SEQ ID NO: 388 - DNA, 55 bp, synthetic construct
  misc_feature 1..55: note = Oligo
  source 1..55: mol_type = other DNA
ttctaatacgactcactatagaggcattggattcgggccaggttttagagctaga

SEQ ID NO: 389 - DNA, 55 bp, synthetic construct
  misc_feature 1..55: note = Oligo
  source 1..55: mol_type = other DNA
ttctaatacgactcactatagaatacgtgttctggaaacccgttttagagctaga

SEQ ID NO: 390 - DNA, 55 bp, synthetic construct
  misc_feature 1..55: note = Oligo
  source 1..55: mol_type = other DNA
ttctaatacgactcactataggtttttaaagcggcacggacgttttagagctaga

SEQ ID NO: 391 - DNA, 55 bp, synthetic construct
  misc_feature 1..55: note = Oligo
  source 1..55: mol_type = other DNA
ttctaatacgactcactatagaacataaagagaaagaccctgttttagagctaga

SEQ ID NO: 392 - DNA, 55 bp, synthetic construct
  misc_feature 1..55: note = Oligo
  source 1..55: mol_type = other DNA
ttctaatacgactcactatagaagccgaaccattcgaggcggttttagagctaga

SEQ ID NO: 393 - DNA, 55 bp, synthetic construct
  misc_feature 1..55: note = Oligo
  source 1..55: mol_type = other DNA
ttctaatacgactcactataggtatttatcaaaccgggcaggttttagagctaga

SEQ ID NO: 394 - DNA, 55 bp, synthetic construct
  misc_feature 1..55: note = Oligo
  source 1..55: mol_type = other DNA
ttctaatacgactcactatagaatgaataaagcgctctccggttttagagctaga

SEQ ID NO: 395 - DNA, 55 bp, synthetic construct
  misc_feature 1..55: note = Oligo
  source 1..55: mol_type = other DNA
ttctaatacgactcactatagactcagcaattacgccccgggttttagagctaga

SEQ ID NO: 396 - DNA, 55 bp, synthetic construct
  misc_feature 1..55: note = Oligo
  source 1..55: mol_type = other DNA
ttctaatacgactcactatagcccgtgaagtggcagaggtcgttttagagctaga

SEQ ID NO: 397 - DNA, 55 bp, synthetic construct
  misc_feature 1..55: note = Oligo
  source 1..55: mol_type = other DNA
ttctaatacgactcactatagccaatccattctgtcagcccgttttagagctaga

SEQ ID NO: 398 - DNA, 55 bp, synthetic construct
  misc_feature 1..55: note = Oligo
  source 1..55: mol_type = other DNA
ttctaatacgactcactatagcaccgagtatgtcagaccgcgttttagagctaga

SEQ ID NO: 399 - DNA, 55 bp, synthetic construct
  misc_feature 1..55: note = Oligo
  source 1..55: mol_type = other DNA
ttctaatacgactcactatagtgcttggaaagttcgagacagttttagagctaga

SEQ ID NO: 400 - DNA, 55 bp, synthetic construct
  misc_feature 1..55: note = Oligo
  source 1..55: mol_type = other DNA
ttctaatacgactcactataggaaaatgaagaacgcgcggggttttagagctaga

SEQ ID NO: 401 - DNA, 55 bp, synthetic construct
  misc_feature 1..55: note = Oligo
  source 1..55: mol_type = other DNA
ttctaatacgactcactatagtaaatcttcaaactgcggacgttttagagctaga

SEQ ID NO: 402 - DNA, 55 bp, synthetic construct
  misc_feature 1..55: note = Oligo
  source 1..55: mol_type = other DNA
ttctaatacgactcactataggaagcaaaagcacttccgccgttttagagctaga

SEQ ID NO: 403 - DNA, 55 bp, synthetic construct
  misc_feature 1..55: note = Oligo
  source 1..55: mol_type = other DNA
ttctaatacgactcactatagtatatgaaaaatcatgtccggttttagagctaga

SEQ ID NO: 404 - DNA, 55 bp, synthetic construct
  misc_feature 1..55: note = Oligo
  source 1..55: mol_type = other DNA
ttctaatacgactcactataggtgctagtgacttcggggccgttttagagctaga

SEQ ID NO: 405 - DNA, 55 bp, synthetic construct
  misc_feature 1..55: note = Oligo
  source 1..55: mol_type = other DNA
ttctaatacgactcactatagtagtgaattagatagggtacgttttagagctaga

SEQ ID NO: 406 - DNA, 55 bp, synthetic construct
  misc_feature 1..55: note = Oligo
  source 1..55: mol_type = other DNA
ttctaatacgactcactatagtattgctggtgcaggggggggttttagagctaga

SEQ ID NO: 407 - DNA, 55 bp, synthetic construct
  misc_feature 1..55: note = Oligo
  source 1..55: mol_type = other DNA
ttctaatacgactcactatagcaattgtgccaccacgtccggttttagagctaga

SEQ ID NO: 408 - DNA, 55 bp, synthetic construct
  misc_feature 1..55: note = Oligo
  source 1..55: mol_type = other DNA
ttctaatacgactcactatagtggcgtaagtggaacgggtcgttttagagctaga

SEQ ID NO: 409 - DNA, 55 bp, synthetic construct
  misc_feature 1..55: note = Oligo
  source 1..55: mol_type = other DNA
ttctaatacgactcactatagtctgcatatctgccctccctgttttagagctaga

SEQ ID NO: 410 - DNA, 55 bp, synthetic construct
  misc_feature 1..55: note = Oligo
  source 1..55: mol_type = other DNA
ttctaatacgactcactatagcaattgatattcgccccccggttttagagctaga

SEQ ID NO: 411 - DNA, 55 bp, synthetic construct
  misc_feature 1..55: note = Oligo
  source 1..55: mol_type = other DNA
ttctaatacgactcactatagattcagctgtggcaggacaggttttagagctaga

SEQ ID NO: 412 - DNA, 55 bp, synthetic construct
  misc_feature 1..55: note = Oligo
  source 1..55: mol_type = other DNA
ttctaatacgactcactatagagtgccggataacgtccggggttttagagctaga

SEQ ID NO: 413 - DNA, 55 bp, synthetic construct
  misc_feature 1..55: note = Oligo
  source 1..55: mol_type = other DNA
ttctaatacgactcactatagtgctgatgttcaaggctcctgttttagagctaga

SEQ ID NO: 414 - DNA, 55 bp, synthetic construct
  misc_feature 1..55: note = Oligo
  source 1..55: mol_type = other DNA
ttctaatacgactcactataggtcaaatcaggtgagctcacgttttagagctaga

SEQ ID NO: 415 - DNA, 55 bp, synthetic construct
  misc_feature 1..55: note = Oligo
  source 1..55: mol_type = other DNA
ttctaatacgactcactatagacgaccatggttgccgccccgttttagagctaga

SEQ ID NO: 416 - DNA, 55 bp, synthetic construct
  misc_feature 1..55: note = Oligo
  source 1..55: mol_type = other DNA
ttctaatacgactcactatagatgtcaaaggtagcccgccggttttagagctaga

SEQ ID NO: 417 - DNA, 55 bp, synthetic construct
  misc_feature 1..55: note = Oligo
  source 1..55: mol_type = other DNA
ttctaatacgactcactataggcgacatccgccataggcccgttttagagctaga

SEQ ID NO: 418 - DNA, 55 bp, synthetic construct
  misc_feature 1..55: note = Oligo
  source 1..55: mol_type = other DNA
ttctaatacgactcactatagttatgggggagagcgaggtcgttttagagctaga

SEQ ID NO: 419 - DNA, 55 bp, synthetic construct
  misc_feature 1..55: note = Oligo
  source 1..55: mol_type = other DNA
ttctaatacgactcactatagcatcagttacgagggcgcgtgttttagagctaga

SEQ ID NO: 420 - DNA, 55 bp, synthetic construct
  misc_feature 1..55: note = Oligo
  source 1..55: mol_type = other DNA
ttctaatacgactcactatagccttcaacttcacccgggcggttttagagctaga

SEQ ID NO: 421 - DNA, 23 bp, synthetic construct
  misc_feature 1..23: note = Oligo
  source 1..23: mol_type = other DNA
aaaaattgctgcatcttctttga

SEQ ID NO: 422 - DNA, 23 bp, synthetic construct
  misc_feature 1..23: note = Oligo
  source 1..23: mol_type = other DNA
aaaaattgctgcatcttctttgg

SEQ ID NO: 423 - DNA, 23 bp, synthetic construct
  misc_feature 1..23: note = Oligo
  source 1..23: mol_type = other DNA
aaccattcaaacggcgattgcgg

SEQ ID NO: 424 - DNA, 23 bp, synthetic construct
  misc_feature 1..23: note = Oligo
  source 1..23: mol_type = other DNA
cactattcaaacggctattgctg

SEQ ID NO: 425 - DNA, 23 bp, synthetic construct
  misc_feature 1..23: note = Oligo
  source 1..23: mol_type = other DNA
aatatccttgccttgagagaacg

SEQ ID NO: 426 - DNA, 23 bp, synthetic construct
  misc_feature 1..23: note = Oligo
  source 1..23: mol_type = other DNA
aatatccttgccttgagagaagg

SEQ ID NO: 427 - DNA, 20 bp, synthetic construct
  misc_feature 1..20: note = Oligo
  source 1..20: mol_type = other DNA
aaaaattgctgcatcttctt

SEQ ID NO: 428 - DNA, 20 bp, synthetic construct
  misc_feature 1..20: note = Oligo
  source 1..20: mol_type = other DNA
aaccattcaaacggcgattg

SEQ ID NO: 429 - DNA, 20 bp, synthetic construct
  misc_feature 1..20: note = Oligo
  source 1..20: mol_type = other DNA
aatatccttgccttgagaga

SEQ ID NO: 430 - DNA, 23 bp, synthetic construct
  misc_feature 1..23: note = Oligo
  misc_feature 21: note = n is a, c, g, or t
  source 1..23: mol_type = other DNA
gctttttaggatatcgtcccngg

SEQ ID NO: 431 - DNA, 23 bp, synthetic construct
  misc_feature 1..23: note = Oligo
  source 1..23: mol_type = other DNA
gctttttaagatatcgtcccagg

SEQ ID NO: 432 - DNA, 23 bp, synthetic construct
  misc_feature 1..23: note = Oligo
  misc_feature 21: note = n is a, c, g, or t
  source 1..23: mol_type = other DNA
gcggtatccacccccactgcngg

SEQ ID NO: 433 - DNA, 23 bp, synthetic construct
  misc_feature 1..23: note = Oligo
  source 1..23: mol_type = other DNA
gcagtatccacccccactgcagg

SEQ ID NO: 434 - DNA, 23 bp, synthetic construct
  misc_feature 1..23: note = Oligo
  misc_feature 21: note = n is a, c, g, or t
  source 1..23: mol_type = other DNA
gttacattacacacaaacttngg

SEQ ID NO: 435 - DNA, 23 bp, synthetic construct
  misc_feature 1..23: note = Oligo
  source 1..23: mol_type = other DNA
gttacattacacacaaattttgg

SEQ ID NO: 436 - DNA, 23 bp, synthetic construct
  misc_feature 1..23: note = Oligo
  misc_feature 21: note = n is a, c, g, or t
  source 1..23: mol_type = other DNA
ggggcgtaaattcttaacatngg

SEQ ID NO: 437 - DNA, 23 bp, synthetic construct
  misc_feature 1..23: note = Oligo
  source 1..23: mol_type = other DNA
ggagcgtaaattcttaacattgg

SEQ ID NO: 438 - DNA, 23 bp, synthetic construct
  misc_feature 1..23: note = Oligo
  misc_feature 21: note = n is a, c, g, or t
  source 1..23: mol_type = other DNA
cgaagggataaatattgcgangg

SEQ ID NO: 439 - DNA, 23 bp, synthetic construct
  misc_feature 1..23: note = Oligo
  source 1..23: mol_type = other DNA
tgaagggataaatattgcgatgg

SEQ ID NO: 440 - DNA, 23 bp, synthetic construct
  misc_feature 1..23: note = Oligo
  misc_feature 21: note = n is a, c, g, or t
  source 1..23: mol_type = other DNA
tagcacttaaaagaggaatgngg

SEQ ID NO: 441 - DNA, 23 bp, synthetic construct
  misc_feature 1..23: note = Oligo
  source 1..23: mol_type = other DNA
tggcacttaaaagaggaatgggg

SEQ ID NO: 442 - DNA, 23 bp, synthetic construct
  misc_feature 1..23: note = Oligo
  misc_feature 21: note = n is a, c, g, or t
  source 1..23: mol_type = other DNA
ttgttttacgatataatacgngg

SEQ ID NO: 443 - DNA, 23 bp, synthetic construct
  misc_feature 1..23: note = Oligo
  source 1..23: mol_type = other DNA
ttgttttgcgatataatacgagg

SEQ ID NO: 444 - DNA, 23 bp, synthetic construct
  misc_feature 1..23: note = Oligo
  misc_feature 21: note = n is a, c, g, or t
  source 1..23: mol_type = other DNA
taatcaagcattagatagctngg

SEQ ID NO: 445 - DNA, 23 bp, synthetic construct
  misc_feature 1..23: note = Oligo
  source 1..23: mol_type = other DNA
gcgtaaagcattagatagcttgg

SEQ ID NO: 446 - DNA, 21 bp, synthetic construct
  misc_feature 1..21: note = Oligo
  source 1..21: mol_type = other DNA
ttctaatacgactcactatag

SEQ ID NO: 447 - DNA, 14 bp, synthetic construct
  misc_feature 1..14: note = Oligo
  source 1..14: mol_type = other DNA
gttttagagctaga

SEQ ID NO: 448 - DNA, 80 bp, synthetic construct
  misc_feature 1..80: note = Oligo
  source 1..80: mol_type = other DNA
aaaagcaccgactcggtgccactttttcaagttgataacggactagccttattttaacttgctatttctagctctaaaac

SEQ ID NO: 449 - DNA, 55 bp, synthetic construct
  misc_feature 1..55: note = Oligo
  source 1..55: mol_type = other DNA
ttctaatacgactcactatagagaatctgcaagtggatattgttttagagctaga

SEQ ID NO: 450 - DNA, 20 bp, synthetic construct
  misc_feature 1..20: note = Oligo
  source 1..20: mol_type = other DNA
agaatctgcaagtggatatt

SEQ ID NO: 451 - DNA, 13 bp, synthetic construct
  misc_feature 1..13: note = Oligo
  source 1..13: mol_type = other DNA
aaaagcaccgact

SEQ ID NO: 452 - DNA, 27 bp, synthetic construct
  misc_feature 1..27: note = Oligo
  source 1..27: mol_type = other DNA
ttaacttgctatttctagctctaaaac

SEQ ID NO: 453 - DNA, 14 bp, synthetic construct
  misc_feature 1..14: note = Oligo
  source 1..14: mol_type = other DNA
tctagctctaaaac

SEQ ID NO: 454 - DNA, 68 bp, synthetic construct
  misc_feature 1..68: note = Oligo
  source 1..68: mol_type = other DNA
ttctaatacgactcactatagagaatctgcaagtggatattgttttagagctagaaatagcaagttaa

SEQ ID NO: 455 - DNA, 13 bp, synthetic construct
  misc_feature 1..13: note = Oligo
  source 1..13: mol_type = other DNA
aatagcaagttaa

SEQ ID NO: 456 - DNA, 13 bp, synthetic construct
  misc_feature 1..13: note = Oligo
  source 1..13: mol_type = other DNA
tgtcggtgctttt

SEQ ID NO: 457 - DNA, 68 bp, synthetic construct
  misc_feature 1..68: note = Oligo
  source 1..68: mol_type = other DNA
ttaacttgctatttctagctctaaaacaatatccacttgcagattctctatagtgagtcgtattagaa

SEQ ID NO: 458 - DNA, 41 bp, synthetic construct
  misc_feature 1..41: note = Oligo
  source 1..41: mol_type = other DNA
aatatccacttgcagattctctatagtgagtcgtattagaa

SEQ ID NO: 459 - RNA, 48 nt, synthetic construct
  misc_feature 1..48: note = Oligo
  source 1..48: mol_type = other RNA
gagaatctgcaagtggatattgttttagagctagaaatagcaagttaa

SEQ ID NO: 460 - RNA, 13 nt, synthetic construct
  misc_feature 1..13: note = Oligo
  source 1..13: mol_type = other RNA
agtcggtgctttt

SEQ ID NO: 461 - RNA, 21 nt, synthetic construct
  misc_feature 1..21: note = Oligo
  source 1..21: mol_type = other RNA
gagaatctgcaagtggatatt

SEQ ID NO: 462 - DNA, 10 bp, synthetic construct
  misc_feature 1..10: note = Oligo
  source 1..10: mol_type = other DNA
gctcttcaac

SEQ ID NO: 463 - DNA, 10 bp, synthetic construct
  misc_feature 1..10: note = Oligo
  source 1..10: mol_type = other DNA
gctcttctag

SEQ ID NO: 464 - DNA, 10 bp, synthetic construct
  misc_feature 1..10: note = Oligo
  source 1..10: mol_type = other DNA
gctcttcgac

SEQ ID NO: 465 - DNA, 10 bp, synthetic construct
  misc_feature 1..10: note = Oligo
  source 1..10: mol_type = other DNA
gctcttcgcg

SEQ ID NO: 466 - DNA, 10 bp, synthetic construct
  misc_feature 1..10: note = Oligo
  source 1..10: mol_type = other DNA
gctcttctcg

SEQ ID NO: 467 - DNA, 10 bp, synthetic construct
  misc_feature 1..10: note = Oligo
  source 1..10: mol_type = other DNA
gctcttcaac

SEQ ID NO: 468 - DNA, 10 bp, synthetic construct
  misc_feature 1..10: note = Oligo
  source 1..10: mol_type = other DNA
gctcttcgac

SEQ ID NO: 469 - DNA, 10 bp, synthetic construct
  misc_feature 1..10: note = Oligo
  source 1..10: mol_type = other DNA
gctcttcacc

SEQ ID NO: 470 - DNA, 10 bp, synthetic construct
  misc_feature 1..10: note = Oligo
  source 1..10: mol_type = other DNA
gctcttccac

SEQ ID NO: 471 - DNA, 10 bp, synthetic construct
  misc_feature 1..10: note = Oligo
  source 1..10: mol_type = other DNA
gctcttccgc